SEQUENCE LISTING (272 sequences)

SEQ ID NO: 1 - protein, 13 aa, Artificial sequence
  Diabetes-associated autoantigenic peptide
MNILLQYVVKSFD

SEQ ID NO: 2 - protein, 9 aa, Artificial sequence
  Diabetes-associated autoantigenic peptide
IAPVFVLLE

SEQ ID NO: 3 - protein, 14 aa, Artificial sequence
  Diabetes-associated autoantigenic peptide
LPRLIAFTSEHSHF

SEQ ID NO: 4 - protein, 13 aa, Artificial sequence
  Diabetes-associated autoantigenic peptide
IAFTSEHSHFSLK

SEQ ID NO: 5 - protein, 14 aa, Artificial sequence
  Diabetes-associated autoantigenic peptide
TVYGAFDPLLAVAD

SEQ ID NO: 6 - protein, 15 aa, Artificial sequence
  Diabetes-associated autoantigenic peptide
KYKIWMHVDAAWGGG

SEQ ID NO: 7 - protein, 15 aa, Artificial sequence
  Diabetes-associated autoantigenic peptide
KHKWKLSGVERANSV

SEQ ID NO: 8 - protein, 15 aa, Artificial sequence
  Diabetes-associated autoantigenic peptide
LYNIIKNREGYEMVF

SEQ ID NO: 9 - protein, 15 aa, Artificial sequence
  Diabetes-associated autoantigenic peptide
PSLRTLEDNEERMSR

SEQ ID NO: 10 - protein, 15 aa, Artificial sequence
  Diabetes-associated autoantigenic peptide
RMMEYGTTMVSYQPL

SEQ ID NO: 11 - protein, 15 aa, Artificial sequence
  Diabetes-associated autoantigenic peptide
SYQPLGDKVNFFRMV

SEQ ID NO: 12 - protein, 13 aa, Artificial sequence
  Diabetes-associated autoantigenic peptide
NFFRMVISNPAAT

SEQ ID NO: 13 - protein, 15 aa, Artificial sequence
  Diabetes-associated autoantigenic peptide
ATHQDIDFLIEEIER

SEQ ID NO: 14 - protein, 9 aa, Artificial sequence
  Diabetes-associated autoantigenic peptide
ATDLLPACD

SEQ ID NO: 15 - protein, 15 aa, Artificial sequence
  Diabetes-associated autoantigenic peptide
FDRSTKVIDFHYPNE

SEQ ID NO: 16 - protein, 9 aa, Artificial sequence
  Diabetes-associated autoantigenic peptide
ELLQEYNWE

SEQ ID NO: 17 - protein, 9 aa, Artificial sequence
  Diabetes-associated autoantigenic peptide
EYNWELADQ

SEQ ID NO: 18 - protein, 9 aa, Artificial sequence
  Diabetes-associated autoantigenic peptide
DIDFLIEEI

SEQ ID NO: 19 - protein, 15 aa, Artificial sequence
  Diabetes-associated autoantigenic peptide
TGHPRYFNQLSTGLD

SEQ ID NO: 20 - protein, 15 aa, Artificial sequence
  Diabetes-associated autoantigenic peptide
TYEIAPVFVLLEYVT

SEQ ID NO: 21 - protein, 9 aa, Artificial sequence
  Diabetes-associated autoantigenic peptide
YVTLKKMRE

SEQ ID NO: 22 - protein, 20 aa, Artificial sequence
  Diabetes-associated autoantigenic peptide
PGGSGDGIFSPGGAISNMYA

SEQ ID NO: 23 - protein, 20 aa, Artificial sequence
  Diabetes-associated autoantigenic peptide
NMYAMMIARFKMFPEVKEKG

SEQ ID NO: 24 - protein, 20 aa, Artificial sequence
  Diabetes-associated autoantigenic peptide
PEVKEKGMAALPRLIAFTSE

SEQ ID NO: 25 - protein, 9 aa, Artificial sequence
  Diabetes-associated autoantigenic peptide
DSVILIKCD

SEQ ID NO: 26 - protein, 9 aa, Artificial sequence
  Diabetes-associated autoantigenic peptide
GKMIPSDLE

SEQ ID NO: 27 - protein, 9 aa, Artificial sequence
  Diabetes-associated autoantigenic peptide
ERRILEAKQ

SEQ ID NO: 28 - protein, 9 aa, Artificial sequence
  Diabetes-associated autoantigenic peptide
ERANSVTWN

SEQ ID NO: 29 - protein, 9 aa, Artificial sequence
  Diabetes-associated autoantigenic peptide
QCSALLVRE

SEQ ID NO: 30 - protein, 15 aa, Artificial sequence
  Diabetes-associated autoantigenic peptide
KHYDLSYDTGDKALQ

SEQ ID NO: 31 - protein, 15 aa, Artificial sequence
  Diabetes-associated autoantigenic peptide
AKGTTGFEAHVDKCL

SEQ ID NO: 32 - protein, 20 aa, Artificial sequence
  Diabetes-associated autoantigenic peptide
VDKCLELAEYLYNIIKNREG

SEQ ID NO: 33 - protein, 9 aa, Artificial sequence
  Diabetes-associated autoantigenic peptide
IIKNREGYE

SEQ ID NO: 34 - protein, 15 aa, Artificial sequence
  Diabetes-associated autoantigenic peptide
MVFDGKPQHTNVCFW

SEQ ID NO: 35 - protein, 15 aa, Artificial sequence
  Diabetes-associated autoantigenic peptide
CFWYIPPSLRTLEDN

SEQ ID NO: 36 - protein, 13 aa, Artificial sequence
  Diabetes-associated autoantigenic peptide
FWYIPPSLRTLED

SEQ ID NO: 37 - protein, 9 aa, Artificial sequence
  Diabetes-associated autoantigenic peptide
SLRTLEDNE

SEQ ID NO: 38 - protein, 15 aa, Artificial sequence
  Diabetes-associated autoantigenic peptide
ERMSRLSKVAPVIKA

SEQ ID NO: 39 - protein, 15 aa, Artificial sequence
  Diabetes-associated autoantigenic peptide
IKARMMEYGTTMVSY

SEQ ID NO: 40 - protein, 15 aa, Artificial sequence
  Diabetes-associated autoantigenic peptide
RMMEYGTTMVSYQPL

SEQ ID NO: 41 - protein, 9 aa, Artificial sequence
  Diabetes-associated autoantigenic peptide
VISNPAATH

SEQ ID NO: 42 - protein, 9 aa, Artificial sequence
  Diabetes-associated autoantigenic peptide
IDFLIEEIE

SEQ ID NO: 43 - protein, 20 aa, Artificial sequence
  Diabetes-associated autoantigenic peptide
NWELADQPQNLEEILMHCQT

SEQ ID NO: 44 - protein, 12 aa, Artificial sequence
  Diabetes-associated autoantigenic peptide
GHPRYFNQLSTG

SEQ ID NO: 45 - protein, 20 aa, Artificial sequence [sequence omitted]

SEQ ID NO: 46 - protein, 14 aa, Artificial sequence
  Diabetes-associated autoantigenic peptide
LTIQIESAADQDPS

SEQ ID NO: 47 - protein, 14 aa, Artificial sequence
  Diabetes-associated autoantigenic peptide
RTGIAQALSSFDLH

SEQ ID NO: 48 - protein, 14 aa, Artificial sequence
  Diabetes-associated autoantigenic peptide
LYPDYQIQAGIMIT

SEQ ID NO: 49 - protein, 14 aa, Artificial sequence
  Diabetes-associated autoantigenic peptide
ILSVHVATAASQDS

SEQ ID NO: 50 - protein, 14 aa, Artificial sequence
  Diabetes-associated autoantigenic peptide
SKRLTFGWYRAEIL

SEQ ID NO: 51 - protein, 14 aa, Artificial sequence
  Diabetes-associated autoantigenic peptide
AILTDAAHLLIDLT

SEQ ID NO: 52 - protein, 14 aa, Artificial sequence
  Diabetes-associated autoantigenic peptide
KATGNRSSKQAHAK

SEQ ID NO: 53 - protein, 14 aa, Artificial sequence
  Diabetes-associated autoantigenic peptide
AVDGVISVHSLHIW

SEQ ID NO: 54 - protein, 21 aa, Artificial sequence
  Diabetes-associated autoantigenic peptide
VSSVSSQFSDAAQASPSSFSD

SEQ ID NO: 55 - protein, 24 aa, Artificial sequence
  Diabetes-associated autoantigenic peptide
LAKEWQALCAYQAEPNTCATAQGE

SEQ ID NO: 56 - protein, 24 aa, Artificial sequence
  Diabetes-associated autoantigenic peptide
KLKVESSPSRSDYINASPIIEHDP

SEQ ID NO: 57 - protein, 20 aa, Artificial sequence
  Diabetes-associated autoantigenic peptide
IKLKVESSPSRSDYINASPI

SEQ ID NO: 58 - protein, 21 aa, Artificial sequence
  Diabetes-associated autoantigenic peptide
MVWESGCTVIVMLTPLVEDGV

SEQ ID NO: 59 - protein, 18 aa, Artificial sequence
  Diabetes-associated autoantigenic peptide
RQHARQQDKERLAALGPE

SEQ ID NO: 60 - protein, 18 aa, Artificial sequence
  Diabetes-associated autoantigenic peptide
GPEGAHGDTTFEYQDLCR

SEQ ID NO: 61 - protein, 18 aa, Artificial sequence
  Diabetes-associated autoantigenic peptide
EGPPEPSRVSSVSSQFSD

SEQ ID NO: 62 - protein, 18 aa, Artificial sequence
  Diabetes-associated autoantigenic peptide
FSDAAQASPSSHSSTPSW

SEQ ID NO: 63 - protein, 18 aa, Artificial sequence
  Diabetes-associated autoantigenic peptide
AEPNTCATAQGEGNIKKN

SEQ ID NO: 64 - protein, 18 aa, Artificial sequence
  Diabetes-associated autoantigenic peptide
NASPIIEHDPRMPAYIAT

SEQ ID NO: 65 - protein, 18 aa, Artificial sequence
  Diabetes-associated autoantigenic peptide
DEGSALYHVYEVNLVSEH

SEQ ID NO: 66 - protein, 18 aa, Artificial sequence
  Diabetes-associated autoanigenic peptides
KGVKEIDIAATLEHVRDQ

SEQ ID NO: 67 - protein, 19 aa, Artificial sequence
  Diabetes-associated autoantigenic peptide
FALTAVAEEVNAILKALPQ

SEQ ID NO: 68 - protein, 15 aa, Artificial sequence
  Diabetes-associated autoantigenic peptide
KNRSLAVLTYDHSRI

SEQ ID NO: 69 - protein, 15 aa, Artificial sequence
  Diabetes-associated autoantigenic peptide
GADPSADATEAYQEL

SEQ ID NO: 70 - protein, 9 aa, Artificial sequence
  Diabetes-associated autoantigenic peptide
EIDIAATLE

SEQ ID NO: 71 - protein, 9 aa, Artificial sequence
  Diabetes-associated autoantigenic peptide
NTCATAQGE

SEQ ID NO: 72 - protein, 9 aa, Artificial sequence
  Diabetes-associated autoantigenic peptide
EPNTCATAQ

SEQ ID NO: 73 - protein, 9 aa, Artificial sequence
  Diabetes-associated autoantigenic peptide
ERLAALGPE

SEQ ID NO: 74 - protein, 9 aa, Artificial sequence
  Diabetes-associated autoantigenic peptide
QHARQQDKE

SEQ ID NO: 75 - protein, 9 aa, Artificial sequence
  Diabetes-associated autoantigenic peptide
YEVNLVSEH

SEQ ID NO: 76 - protein, 9 aa, Artificial sequence
  Diabetes-associated autoantigenic peptide
GASLYHVYE

SEQ ID NO: 77 - protein, 9 aa, Artificial sequence
  Diabetes-associated autoantigenic peptide
FALTAVAEE

SEQ ID NO: 78 - protein, 9 aa, Artificial sequence
  Diabetes-associated autoantigenic peptide
GAHGDTTFE

SEQ ID NO: 79 - protein, 9 aa, Artificial sequence
  Diabetes-associated autoantigenic peptide
GDTTFEYQD

SEQ ID NO: 80 - protein, 9 aa, Artificial sequence
  Diabetes-associated autoantigenic peptide
AAQASPSSH

SEQ ID NO: 81 - protein, 9 aa, Artificial sequence
  Diabetes-associated autoantigenic peptide
SRVSSVSSQ

SEQ ID NO: 82 - protein, 9 aa, Artificial sequence
  Diabetes-associated autoantigenic peptide
TQFHFLSWP

SEQ ID NO: 83 - protein, 9 aa, Artificial sequence
  Diabetes-associated autoantigenic peptide
EEPAQANMD

SEQ ID NO: 84 - protein, 9 aa, Artificial sequence
  Diabetes-associated autoantigenic peptide
GHMILAYME

SEQ ID NO: 85 - protein, 9 aa, Artificial sequence
  Diabetes-associated autoantigenic peptide
MILAYMEDH

SEQ ID NO: 86 - protein, 9 aa, Artificial sequence
  Diabetes-associated autoantigenic peptide
QALCAYQAE

SEQ ID NO: 87 - protein, 9 aa, Artificial sequence
  Diabetes-associated autoantigenic peptide
EWQALCAYQ

SEQ ID NO: 88 - protein, 9 aa, Artificial sequence
  Diabetes-associated autoantigenic peptide
LVRSKDQFE

SEQ ID NO: 89 - protein, 9 aa, Artificial sequence
  Diabetes-associated autoantigenic peptide
VEDGVKQCD

SEQ ID NO: 90 - protein, 9 aa, Artificial sequence
  Diabetes-associated autoantigenic peptide
YILIDMVLN

SEQ ID NO: 91 - protein, 9 aa, Artificial sequence
  Diabetes-associated autoantigenic peptide
ESGCTVIVM

SEQ ID NO: 92 - protein, 9 aa, Artificial sequence
  Diabetes-associated autoantigenic peptide
LCAYQAEPN

SEQ ID NO: 93 - protein, 9 aa, Artificial sequence
  Diabetes-associated autoantigenic peptide
ETRTLTQFH

SEQ ID NO: 94 - protein, 9 aa, Artificial sequence
  Diabetes-associated autoantigenic peptide
VESSPSRSD

SEQ ID NO: 95 - protein, 9 aa, Artificial sequence
  Diabetes-associated autoantigenic peptide
GPLSHTIAD

SEQ ID NO: 96 - protein, 9 aa, Artificial sequence
  Diabetes-associated autoantigenic peptide
SLFNRAEGP

SEQ ID NO: 97 - protein, 9 aa, Artificial sequence
  Diabetes-associated autoantigenic peptide
HPDFLPYDH

SEQ ID NO: 98 - protein, 9 aa, Artificial sequence
  Diabetes-associated autoantigenic peptide
HFLSWPAEG

SEQ ID NO: 99 - protein, 9 aa, Artificial sequence
  Diabetes-associated autoantigenic peptide
DFRRKVNKC

SEQ ID NO: 100 - protein, 9 aa, Artificial sequence
  Diabetes-associated autoantigenic peptide
HCSDGAGRT

SEQ ID NO: 101 - protein, 9 aa, Artificial sequence
  Diabetes-associated autoantigenic peptide
LVRSFYLKN

SEQ ID NO: 102 - protein, 15 aa, Artificial sequence
  Diabetes-associated autoantigenic peptide
KNRSLAVLTYDHSRI

SEQ ID NO: 103 - protein, 15 aa, Artificial sequence
  Diabetes-associated autoantigenic peptide
GADPSADATEAYQEL

SEQ ID NO: 104 - protein, 16 aa, Artificial sequence
  Diabetes-associated autoantigenic peptide
ANMDISTGHMILAYME

SEQ ID NO: 105 - protein, 16 aa, Artificial sequence
  Diabetes-associated autoantigenic peptide
WQALCAYQAEPNTCAT

SEQ ID NO: 106 - protein, 16 aa, Artificial sequence
  Diabetes-associated autoantigenic peptide
LSHTIADFWQMVWESG

SEQ ID NO: 107 - protein, 16 aa, Artificial sequence
  Diabetes-associated autoantigenic peptide
DFWQMVWESGCTVIVM

SEQ ID NO: 108 - protein, 16 aa, Artificial sequence
  Diabetes-associated autoantigenic peptide
WESGCTVIVMLTPLVE

SEQ ID NO: 109 - protein, 16 aa, Artificial sequence
  Diabetes-associated autoantigenic peptide
VIVMLTPLVEDGVKQC

SEQ ID NO: 110 - protein, 16 aa, Artificial sequence
  Diabetes-associated autoantigenic peptide
SEHIWCEDFLVRSFYL

SEQ ID NO: 111 - protein, 16 aa, Artificial sequence
  Diabetes-associated autoantigenic peptide
WCEDFLVRSFYLKNVQ

SEQ ID NO: 112 - protein, 16 aa, Artificial sequence
  Diabetes-associated autoantigenic peptide
EDFLVRSFYLKNVQTQ

SEQ ID NO: 113 - protein, 16 aa, Artificial sequence
  Diabetes-associated autoantigenic peptide
DFRRKVNKCYRGRSCP

SEQ ID NO: 114 - protein, 16 aa, Artificial sequence
  Diabetes-associated autoantigenic peptide
YILIDMVLNRMAKGVK

SEQ ID NO: 115 - protein, 16 aa, Artificial sequence
  Diabetes-associated autoantigenic peptide
FEFALTAVAEEVNAIL

SEQ ID NO: 116 - protein, 9 aa, Artificial sequence
  Diabetes-associated autoantigenic peptide
EALYLVCGE

SEQ ID NO: 117 - protein, 9 aa, Artificial sequence
  Diabetes-associated autoantigenic peptide
SICSLYQLE

SEQ ID NO: 118 - protein, 9 aa, Artificial sequence
  Diabetes-associated autoantigenic peptide
ALLALWGPD

SEQ ID NO: 119 - protein, 9 aa, Artificial sequence
  Diabetes-associated autoantigenic peptide
GSLQPLALE

SEQ ID NO: 120 - protein, 9 aa, Artificial sequence
  Diabetes-associated autoantigenic peptide
TPKTRREAE

SEQ ID NO: 121 - protein, 9 aa, Artificial sequence
  Diabetes-associated autoantigenic peptide
PAAAFVNQH

SEQ ID NO: 122 - protein, 9 aa, Artificial sequence
  Diabetes-associated autoantigenic peptide
DPAAAFVNQ

SEQ ID NO: 123 - protein, 9 aa, Artificial sequence
  Diabetes-associated autoantigenic peptide
PDPAAAFVN

SEQ ID NO: 124 - protein, 9 aa, Artificial sequence
  Diabetes-associated autoantigenic peptide
QKRGIVEQC

SEQ ID NO: 125 - protein, 9 aa, Artificial sequence
  Diabetes-associated autoantigenic peptide
ELGGGPGAG

SEQ ID NO: 126 - protein, 9 aa, Artificial sequence
  Diabetes-associated autoantigenic peptide
EAEDLQVGQ

SEQ ID NO: 127 - protein, 9 aa, Artificial sequence
  Diabetes-associated autoantigenic peptide
LQVGQVELG

SEQ ID NO: 128 - protein, 9 aa, Artificial sequence
  Diabetes-associated autoantigenic peptide
HLCGSHLVE

SEQ ID NO: 129 - protein, 12 aa, Artificial sequence
  Diabetes-associated autoantigenic peptide
GIVEQCCTSICS

SEQ ID NO: 130 - protein, 14 aa, Artificial sequence
  Diabetes-associated autoantigenic peptide
KRGIVEQCCTSICS

SEQ ID NO: 131 - protein, 16 aa, Artificial sequence
  Diabetes-associated autoantigenic peptide
LALLALWGPDPAAAFV

SEQ ID NO: 132 - protein, 16 aa, Artificial sequence
  Diabetes-associated autoantigenic peptide
PAAAFVNQHLCGSHLV

SEQ ID NO: 133 - protein, 15 aa, Artificial sequence
  Diabetes-associated autoantigenic peptide
SHLVEALYLVCGERG

SEQ ID NO: 134 - protein, 13 aa, Artificial sequence
  Diabetes-associated autoantigenic peptide
FFYTPKTRREAED

SEQ ID NO: 135 - protein, 18 aa, Artificial sequence
  Diabetes-associated autoantigenic peptide
GAGSLQPLALEGSLQKRG

SEQ ID NO: 136 - protein, 17 aa, Artificial sequence
  Diabetes-associated autoantigenic peptide
SLQKRGIVEQCCTSICS

SEQ ID NO: 137 - protein, 20 aa, Artificial sequence
  Diabetes-associated autoantigenic peptide
KFGADARALMLQGVDLLADA

SEQ ID NO: 138 - protein, 20 aa, Artificial sequence
  Diabetes-associated autoantigenic peptide
NPVEIRRGVMLAVDAVIAEL

SEQ ID NO: 139 - protein, 21 aa, Artificial sequence
  Diabetes-associated autoantigenic peptide
QSIVPALEIANAHRKPLVIIA

SEQ ID NO: 140 - protein, 20 aa, Artificial sequence
  Diabetes-associated autoantigenic peptide
LVLNRLKVGLQVVAVKAPGF

SEQ ID NO: 141 - protein, 20 aa, Artificial sequence
  Diabetes-associated autoantigenic peptide
IVLGGGCALLRCIPALDSLT

SEQ ID NO: 142 - protein, 24 aa, Artificial sequence
  Diabetes-associated autoantigenic peptide
VLGGGCALLRCIPALDSLTPANED

SEQ ID NO: 143 - protein, 20 aa, Artificial sequence
  Diabetes-associated autoantigenic peptide
EIIKRTLKIPAMTIAKNAGV

SEQ ID NO: 144 - protein, 20 aa, Artificial sequence
  Diabetes-associated autoantigenic peptide
VNMVEKGIIDPTKVVRTALL

SEQ ID NO: 145 - protein, 20 aa, Artificial sequence
  Diabetes-associated autoantigenic peptide
MAKAAAVGIDLGTTYSCVGV

SEQ ID NO: 146 - protein, 20 aa, Artificial sequence
  Diabetes-associated autoantigenic peptide
GLNVLRIINEPTAAAIAYGL

SEQ ID NO: 147 - protein, 20 aa, Artificial sequence
  Diabetes-associated autoantigenic peptide
TIDDGIFEVKATAGDTHLGG

SEQ ID NO: 148 - protein, 20 aa, Artificial sequence
  Diabetes-associated autoantigenic peptide
THLGGEDFDNRLVNHFVEEF

SEQ ID NO: 149 - protein, 20 aa, Artificial sequence
  Diabetes-associated autoantigenic peptide
KRTLSSSTQASLEIDSLFEG

SEQ ID NO: 150 - protein, 20 aa, Artificial sequence
  Diabetes-associated autoantigenic peptide
LLLLDVAPLSLGLETAGGVM

SEQ ID NO: 151 - protein, 20 aa, Artificial sequence
  Diabetes-associated autoantigenic peptide
PTKQTQIFTTYSDNQPGVLI

SEQ ID NO: 152 - protein, 20 aa, Artificial sequence
  Diabetes-associated autoantigenic peptide
KANKITITNDKGRLSKEEIE

SEQ ID NO: 153 - protein, 20 aa, Artificial sequence
  Diabetes-associated autoantigenic peptide
KEEIERMVQEAEKYKAEDEV

SEQ ID NO: 154 - protein, 13 aa, Artificial sequence
  Diabetes-associated autoantigenic peptide
QHLQKDYRAYYTF

SEQ ID NO: 155 - protein, 13 aa, Artificial sequence
  Diabetes-associated autoantigenic peptide
RVLNIDLLWSVPI

SEQ ID NO: 156 - protein, 13 aa, Artificial sequence
  Diabetes-associated autoantigenic peptide
YTFLNFMSNVGDP

SEQ ID NO: 157 - protein, 13 aa, Artificial sequence
  Diabetes-associated autoantigenic peptide
DWIHIDTTPFAGL

SEQ ID NO: 158 - protein, 215 aa, Artificial sequence
  G3H8 light chain [variable(VL)+ constant (CL) domains] amino acid sequence
LETTLTQSPATLSVSPGERVTLSCRASQSVGSNLAWYQQKFGQAPRLLIYDASTRATGIPARFSGSGSGTEFTLTISRLEPEDFAVYYCHQYGSSPRTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - DNA, 669 bp, Artificial sequence
  G3H8 light chain [variable(VL)+ constant (CL) domains] nucleic acid sequence
cttgaaacgacactcacgcagtctccagccaccctgtctgtgtctccaggggaaagagtcaccctctcctgcagggccagtcagagtgttggcagcaacttagcctggtaccagcagaaatttggccaggctcccaggctcctcatctatgatgcatccaccagggccactggtatcccagccaggttcagtggcagtgggtctgggacagagttcactctcaccatcagcagactggagcctgaagattttgcagtgtattactgtcaccagtatggtagctcacctcggacgttcggccaagggaccaaggtggacatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacggagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataaggcgcgccaattctattt

SEQ ID NO: 160 - protein, 254 aa, Artificial sequence
  G3H8 heavy chain [variable(VH)+ constant 1(CH1) domains] amino acid sequence
QVQLVQSGAEVKKPGASVKVSCKASGYTFTTYGISWVRQAPGQGLEWMGWISAYNGHTNYAQMLQGRVTMTTDTSTSTAYMELRGLRSDDTAVYYCAREAYASYGSGSYWTDYWGQGTLVTVSSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAHHHHHHGAAEQKLISEEDLNGAA

SEQ ID NO: 161 - DNA, 759 bp, Artificial sequence
  G3H8 heavy chain [variable(VH)+ constant 1(CH1) domains] nucleic acid sequence
caggtccagctggtacagtctggggctgaggtgaagaagcctggggcctcagtgaaggtctcctgcaaggcttctggttacacctttaccacctatggtatcagctgggtgcgacaggcccctggacaagggcttgagtggatgggatggatcagcgcttacaatggtcacacaaactatgcacagatgctccagggcagagtcaccatgaccacagacacatccacgagcacagcctacatggagctgaggggcctgagatctgacgacacggccgtgtattactgtgcgagagaggcctatgcttcctatggttcggggagttattggactgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgcggccgcacatcatcatcaccatcacggggccgcagaacaaaaactcatctcagaagaggatctgaatggggccgca

SEQ ID NO: 162 - protein, 216 aa, Artificial sequence
  G1H12 light chain [variable(VL)+ constant (CL) domains] amino acid sequence
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSVWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 163 - DNA, 654 bp, Artificial sequence
  G1H12 light chain [variable(VL)+ constant (CL) domains] nucleic acid sequence
cagtctgtgctgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagtgtctgggtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcataataa

SEQ ID NO: 164 - protein, 252 aa, Artificial sequence
  G1H12 heavy chain [variable(VH)+ constant 1(CH1) domains] amino acid sequence
QVQLVQSGAEVKKPGASVKVSCKASGYTFTSYGISWVRQAPGQGLEWMGGIIPIFGTANYAQKFQGRVTITADKSTSTAYMELSSLRSEDTAVYYCARDPQSYYYDSSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAHHHHHHGAAEQKLISEEDLNGAA

SEQ ID NO: 165 - DNA, 756 bp, Artificial sequence
  G1H12 heavy chain [variable(VH)+ constant 1(CH1) domains] nucleic acid sequence
caggtccagctggtgcagtctggagctgaggtgaagaagcctggggcctcagtgaaggtctcctgcaaggcttctggttacacctttaccagctatggtatcagctgggtgcgacaggcccctggacaagggcttgagtggatgggagggatcatccctatctttggtacagcaaactacgcacagaagttccagggcagagtcacgattaccgcggacaaatccacgagcacagcctacatggagctgagcagcctgagatctgaagacacggctgtgtattactgtgcgagagatccccagtcctattactatgatagtagtggttttgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgcggccgcacatcatcatcaccatcacggggccgcagaacaaaaactcatctcagaagaggatctgaatggggccgca

SEQ ID NO: 166 - protein, 302 aa, Artificial sequence
  Recombinant beta chain (DRB1*0401)
MVCLKFPGGSCMTALTVTLMVLSSPLALAGDTNFFRMVISNPAATGGGSLVPRGSGGGGSRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSKVDGGGGGRIARLEEKVKTLKAQNSELASTANMLREQVAQLKQKVMNH

SEQ ID NO: 167 - protein, 278 aa, Artificial sequence
  Recombinant alpha chain (DRA1*0101)
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVDGGGGGLTDTLQAETDQLEDEKSALQTEIANLLKEKEKLEFILAAHGLNDIFEAQKIEWH

SEQ ID NO: 168 - DNA, 906 bp, Artificial sequence
  Recombinant beta chain (DRB1*0401) nucleic acid sequence
atggtgtgtctgaagttccctggaggctcctgcatgacagcgctgacagtgacactgatggtgctgagctccccactggctttggctggggacaccaacttctttcgtatggttatcagcaatccagctgcgactggtggtggctcactagtgccacggggctctggaggaggtgggtcccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgagtctatcctgaggtgactgtgtatcctgcaaagacccagcccctgcagcaccacaacctcctggtctgctctgtgaatggtttctatccaggcagcattgaagtcaggtggttccggaacggccaggaagagaagactggggtggtgtccacaggcctgatccagaatggagactggaccttccagaccctggtgatgctggaaacagttcctcggagtggagaggtttacacctgccaagtggagcacccaagcctgacgagccctctcacagtggaatggagagcacggtctgaatctgcacagagcaaggtcgacggaggtggcggcggtcgcatcgcccggctcgaggaaaaagtgaaaaccttgaaagctcagaactcggagctggcgtccacggccaacatgctcagggaacaggtggcacagcttaaacagaaagtcatgaaccat

SEQ ID NO: 169 - DNA, 834 bp, Artificial sequence
  Recombinant alpha chain (DRA1*0101) nucleic acid sequence
atggccataagtggagtccctgtgctaggatttttcatcatagctgtgctgatgagcgctcaggaatcatgggctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacgaacagccctgtggaactgagagagcccaacgtcctcatctgtttcatcgacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtcgacggaggtggcggcggtttaactgatacactccaagcggagacagatcaacttgaagacgagaagtctgcgttgcagaccgagattgccaatctactgaaagagaaggaaaaactggagttcatcctggccgcccatggcctgaacgacatcttcgaggcccagaagatcgagtggcac

SEQ ID NO: 170 - protein, 7 aa, Artificial sequence
  Exemplary linker peptide
VDGGGGG

SEQ ID NO: 171 - protein, 11 aa, Artificial sequence
  G3H8 light chain CDR1
RASQSVGSNLA

SEQ ID NO: 172 - protein, 7 aa, Artificial sequence
  G3H8 light chain CDR2
DASTRAT

SEQ ID NO: 173 - protein, 9 aa, Artificial sequence
  G3H8 light chain CDR3
HQYGSSPRT

SEQ ID NO: 174 - DNA, 33 bp, Artificial sequence
  G3H8 light chain CDR1 nucleotide sequence
agggccagtcagagtgttggcagcaacttagcc

SEQ ID NO: 175 - DNA, 21 bp, Artificial sequence
  G3H8 light chain CDR2 nucleotide sequence
gatgcatccaccagggccact

SEQ ID NO: 176 - DNA, 27 bp, Artificial sequence
  G3H8 light chain CDR3 nucleotide sequence
caccagtatggtagctcacctcggacg

SEQ ID NO: 177 - protein, 10 aa, Artificial sequence
  G3H8 heavy chain CDR1
GYTFTTYGIS

SEQ ID NO: 178 - protein, 17 aa, Artificial sequence
  G3H8 heavy chain CDR2
WISAYNGHTNYAQMLQG

SEQ ID NO: 179 - protein, 15 aa, Artificial sequence
  G3H8 heavy chain CDR3
EAYASYGSGSYWTDY

SEQ ID NO: 180 - DNA, 30 bp, Artificial sequence
  G3H8 heavy chain CDR1 nucleotide sequence
ggttacacctttaccacctatggtatcagc

SEQ ID NO: 181 - DNA, 51 bp, Artificial sequence
  G3H8 heavy chain CDR2 nucleotide sequence
tggatcagcgcttacaatggtcacacaaactatgcacagatgctccagggc

SEQ ID NO: 182 - DNA, 45 bp, Artificial sequence
  G3H8 heavy chain CDR3 nucleotide sequence
gaggcctatgcttcctatggttcggggagttattggactgactac

SEQ ID NO: 183 - protein, 13 aa, Artificial sequence
  G1H12 light chain CDR1
SGSSSNIGNNYVS

SEQ ID NO: 184 - protein, 7 aa, Artificial sequence
  G1H12 light chain CDR2
DNNKRPS

SEQ ID NO: 185 - protein, 11 aa, Artificial sequence
  G1H12 light chain CDR3
GTWDSSLSVWV

SEQ ID NO: 186 - DNA, 39 bp, Artificial sequence
  G1H12 light chain CDR1 nucleotide sequence
tctggaagcagctccaacattgggaataattatgtatcc

SEQ ID NO: 187 - DNA, 21 bp, Artificial sequence
  G1H12 light chain CDR2 nucleotide sequence
gacaataataagcgaccctca

SEQ ID NO: 188 - DNA, 33 bp, Artificial sequence
  G1H12 light chain CDR3 nucleotide sequence
ggaacatgggatagcagcctgagtgtctgggtg

SEQ ID NO: 189 - protein, 10 aa, Artificial sequence
  G1H12 heavy chain CDR1
GYTFTSYGIS

SEQ ID NO: 190 - protein, 17 aa, Artificial sequence
  G1H12 heavy chain CDR2
GIIPIFGTANYAQKFQG

SEQ ID NO: 191 - protein, 14 aa, Artificial sequence
  G1H12 heavy chain CDR3
DPQSYYYDSSGFDY

SEQ ID NO: 192 - DNA, 30 bp, Artificial sequence
  G1H12 heavy chain CDR1 nucleotide sequence
ggttacacctttaccagctatggtatcagc

SEQ ID NO: 193 - DNA, 51 bp, Artificial sequence
  G1H12 heavy chain CDR2 nucleotide sequence
gggatcatccctatctttggtacagcaaactacgcacagaagttccagggc

SEQ ID NO: 194 - DNA, 42 bp, Artificial sequence
  G1H12 heavy chain CDR3 nucleotide sequence
gatccccagtcctattactatgatagtagtggttttgactac

SEQ ID NO: 195 - protein, 40 aa, Artificial sequence
  Jun derived peptide
RIARLEEKVKTLKAQNSELASTANMLREQVAQLKQKVMNH

SEQ ID NO: 196 - protein, 40 aa, Artificial sequence
  Fos derived peptide
LTDTLQAETDQLEDEKSALQTEIANLLKEKEKLEFILAAH

SEQ ID NO: 197 - protein, 14 aa, Artificial sequence
  Bir A recognition sequence
LGGIFEAMKMELRD

SEQ ID NO: 198 - DNA, 2824 bp, Homo sapiens
gcggccgcccgcacttcccgcctctggctcgcccgaggacgcgctggcacgcctcccaccccctcactctgactccagctggcgtgcatggtctgcctcgcatcctcacgactcagctccctccctctctcgtgtttttttcctccgccgccccctcattcatccccactgggctccctttccctcaaatgctctggggctctccgcgctttcctgagtccgggctccgaggacccttaggtagtcccggtctcttttaaagctccccggcttccaaagggttgccacgtccctaaaccctgtctccagctcgcatacacacacgcacagacacgcacgttttctgttcctgcgtgacacccgccctcgccgctcggccccgccggtccccgcgcggtgccctcctcccgccacacgggcacgcacgcgcgcgcagggccaagcccgaggcagctcgcccgcagctcgcactcgcaggcgacctgctccagtctccaaagccgatggcatctccgggctctggcttttggtctttcgggtcggaagatggctctggggattccgagaatcccggcacagcgcgagcctggtgccaagtggctcagaagttcacgggcggcatcggaaacaaactgtgcgccctgctctacggagacgccgagaagccggcggagagcggcgggagccaacccccgcgggccgccgcccggaaggccgcctgcgcctgcgaccagaagccctgcagctgctccaaagtggatgtcaactacgcgtttctccatgcaacagacctgctgccggcgtgtgatggagaaaggcccactttggcgtttctgcaagatgttatgaacattttacttcagtatgtggtgaaaagtttcgatagatcaaccaaagtgattgatttccattatcctaatgagcttctccaagaatataattgggaattggcagaccaaccacaaaatttggaggaaattttgatgcattgccaaacaactctaaaatatgcaattaaaacagggcatcctagatacttcaatcaactttctactggtttggatatggttggattagcagcagactggctgacatcaacagcaaatactaacatgttcacctatgaaattgctccagtatttgtgcttttggaatatgtcacactaaagaaaatgagagaaatcattggctggccagggggctctggcgatgggatattttctcccggtggcgccatatctaacatgtatgccatgatgatcgcacgctttaagatgttcccagaagtcaaggagaaaggaatggctgctcttcccaggctcattgccttcacgtctgaacatagtcatttttctctcaagaagggagctgcagccttagggattggaacagacagcgtgattctgattaaatgtgatgagagagggaaaatgattccatctgatcttgaaagaaggattcttgaagccaaacagaaagggtttgttcctttcctcgtgagtgccacagctggaaccaccgtgtacggagcatttgaccccctcttagctgtcgctgacatttgcaaaaagtataagatctggatgcatgtggatgcagcttggggtgggggattactgatgtcccgaaaacacaagtggaaactgagtggcgtggagagggccaactctgtgacgtggaatccacacaagatgatgggagtccctttgcagtgctctgctctcctggttagagaagagggattgatgcagaattgcaaccaaatgcatgcctcctacctctttcagcaagataaacattatgacctgtcctatgacactggagacaaggccttacagtgcggacgccacgttgatgtttttaaactatggctgatgtggagggcaaaggggactaccgggtttgaagcgcatgttgataaatgtttggagttggcagagtatttatacaacatcataaaaaaccgagaaggatatgagatggtgtttgatgggaagcctcagcacacaaatgtctgcttctggtacattcctccaagcttgcgtactctggaagacaatgaagagagaatgagtcgcctctcgaaggtggctccagtgattaaagccagaatgatggagtatggaaccacaatggtcagctaccaacccttgggagacaaggtcaatttcttccgcatggtcatctcaaacccagcggcaactcaccaagacattgacttcctgattgaagaaatagaacgccttggacaagatttataataaccttgctcaccaagctgttccacttctctagagaacatgccctcagctaagccccctactgagaaacttcctttgagaattgtgcgacttcacaaaatgcaaggtgaacaccactttgtctctgagaacagacgttaccaattatggagtgtcaccagctgccaaaatcgtaggtgttggctctgctggtcactggagtagttgctactcttcagaatatggacaaagaaggcacaggtgtaaatatagtagcaggatgaggaacctcaaactgggtatcattttgcacgtgctcttctgttctcaaatgctaaatgcaaacactgtgtatttattagttaggtgtgccaaactaccgttcccaaattggtgtttctgaatgacatcaacattcccccaacattactccattactaaagacagaaaaaaataaaaacataaaatatacaaacatgtggcaacctgttcttcctaccaaatataaacttgtgtatgatccaagtattttatctgtgttgtctctctaaacccaaataaatgtgtaaatgtggacacatctc

SEQ ID NO: 199 - DNA, 2419 bp, Homo sapiens
gcggccgcccgcacttcccgcctctggctcgcccgaggacgcgctggcacgcctcccaccccctcactctgactccagctggcgtgcatggtctgcctcgcatcctcacgactcagctccctccctctctcgtgtttttttcctccgccgccccctcattcatccccactgggctccctttccctcaaatgctctggggctctccgcgctttcctgagtccgggctccgaggacccttaggtagtcccggtctcttttaaagctccccggcttccaaagggttgccacgtccctaaaccctgtctccagctcgcatacacacacgcacagacacgcacgttttctgttcctgcgtgacacccgccctcgccgctcggccccgccggtccccgcgcggtgccctcctcccgccacacgggcacgcacgcgcgcgcagggccaagcccgaggcagctcgcccgcagctcgcactcgcaggcgacctgctccagtctccaaagccgatggcatctccgggctctggcttttggtctttcgggtcggaagatggctctggggattccgagaatcccggcacagcgcgagcctggtgccaagtggctcagaagttcacgggcggcatcggaaacaaactgtgcgccctgctctacggagacgccgagaagccggcggagagcggcgggagccaacccccgcgggccgccgcccggaaggccgcctgcgcctgcgaccagaagccctgcagctgctccaaagtggatgtcaactacgcgtttctccatgcaacagacctgctgccggcgtgtgatggagaaaggcccactttggcgtttctgcaagatgttatgaacattttacttcagtatgtggtgaaaagtttcgatagatcaaccaaagtgattgatttccattatcctaatgagcttctccaagaatataattgggaattggcagaccaaccacaaaatttggaggaaattttgatgcattgccaaacaactctaaaatatgcaattaaaacagggcatcctagatacttcaatcaactttctactggtttggatatggttggattagcagcagactggctgacatcaacagcaaatactaacatgttcacctatgaaattgctccagtatttgtgcttttggaatatgtcacactaaagaaaatgagagaaatcattggctggccagggggctctggcgatgggatattttctcccggtggcgccatatctaacatgtatgccatgatgatcgcacgctttaagatgttcccagaagtcaaggagaaaggaatggctgctcttcccaggctcattgccttcacgtctgaacatagtcatttttctctcaagaagggagctgcagccttagggattggaacagacagcgtgattctgattaaatgtgatgagagagggaaaatgattccatctgatcttgaaagaaggattcttgaagccaaacagaaagggtttgttcctttcctcgtgagtgccacagctggaaccaccgtgtacggagcatttgaccccctcttagctgtcgctgacatttgcaaaaagtataagatctggatgcatgtggatgcagcttggggtgggggattactgatgtcccgaaaacacaagtggaaactgagtggcgtggagagggccaactctgtgacgtggaatccacacaagatgatgggagtccctttgcagtgctctgctctcctggttagagaagagggattgatgcagaattgcaaccaaatgcatgcctcctacctctttcagcaagataaacattatgacctgtcctatgacactggagacaaggccttacagtgcggacgccacgttgatgtttttaaactatggctgatgtggagggcaaaggggactaccgggtttgaagcgcatgttgataaatgtttggagttggcagagtatttatacaacatcataaaaaaccgagaaggatatgagatggtgtttgatgggaagcctcagcacacaaatgtctgcttctggtacattcctccaagcttgcgtactctggaagacaatgaagagagaatgagtcgcctctcgaaggtggctccagtgattaaagccagaatgatggagtatggaaccacaatggtcagctaccaacccttgggagacaaggtcaatttcttccgcatggtcatctcaaacccagcggcaactcaccaagacattgacttcctgattgaagaaatagaacgccttggacaagatttataataaccttgctcaccaagctgttccacttctctaggtagacaattaagttgtcacaaactgtgtgaatgtatttgtagtttgttccaaagtaaatctatttctatattgtggtgtcaaagtagagtttaaaaattaaacaaaaaagacattgctccttt

SEQ ID NO: 200 - protein, 585 aa, Homo sapiens
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 201 - DNA, 3488 bp, Homo sapiens
gcaaaaccgtgagctggatttataatcgccctataaagctccagaggcggtcaggcacctgcagaggagccccgccgctccgccgactagctgcccccgcgagcaacggcctcgtgatttccccgccgatccggtccccgcctccccactctgcccccgcctaccccggagccgtgcagccgcctctccgaatctctctcttctcctggcgctcgcgtgcgagagggaactagcgagaacgaggaagcagctggaggtgacgccgggcagattacgcctgtcagggccgagccgagcggatcgctgggcgctgtgcagaggaaaggcgggagtgcccggctcgctgtcgcagagccgagcctgtttctgcgccggaccagtcgaggactctggacagtagaggccccgggacgaccgagctgatggcgtcttcgaccccatcttcgtccgcaacctcctcgaacgcgggagcggaccccaataccactaacctgcgccccacaacgtacgatacctggtgcggcgtggcccatggatgcaccagaaaactggggctcaagatctgcggcttcttgcaaaggaccaacagcctggaagagaagagtcgccttgtgagtgccttcaaggagaggcaatcctccaagaacctgctttcctgtgaaaacagcgaccgggatgcccgcttccggcgcacagagactgacttctctaatctgtttgctagagatctgcttccggctaagaacggtgaggagcaaaccgtgcaattcctcctggaagtggtggacatactcctcaactatgtccgcaagacatttgatcgctccaccaaggtgctggactttcatcacccacaccagttgctggaaggcatggagggcttcaacttggagctctctgaccaccccgagtccctggagcagatcctggttgactgcagagacaccttgaagtatggggttcgcacaggtcatcctcgatttttcaaccagctctccactggattggatattattggcctagctggagaatggctgacatcaacggccaataccaacatgtttacatatgaaattgcaccagtgtttgtcctcatggaacaaataacacttaagaagatgagagagatagttggatggtcaagtaaagatggtgatgggatattttctcctgggggcgccatatccaacatgtacagcatcatggctgctcgctacaagtacttcccggaagttaagacaaagggcatggcggctgtgcctaaactggtcctcttcacctcagaacagagtcactattccataaagaaagctggggctgcacttggctttggaactgacaatgtgattttgataaagtgcaatgaaagggggaaaataattccagctgattttgaggcaaaaattcttgaagccaaacagaagggatatgttcccttttatgtcaatgcaactgctggcacgactgtttatggagcttttgatccgatacaagagattgcagatatatgtgagaaatataacctttggttgcatgtcgatgctgcctggggaggtgggctgctcatgtccaggaagcaccgccataaactcaacggcatagaaagggccaactcagtcacctggaaccctcacaagatgatgggcgtgctgttgcagtgctctgccattctcgtcaaggaaaagggtatactccaaggatgcaaccagatgtgtgcaggatacctcttccagccagacaagcagtatgatgtctcctacgacaccggggacaaggcaattcagtgtggccgccacgtggatatcttcaagttctggctgatgtggaaagcaaagggcacagtgggatttgaaaaccagatcaacaaatgcctggaactggctgaatacctctatgccaagattaaaaacagagaagaatttgagatggttttcaatggcgagcctgagcacacaaacgtctgtttttggtatattccacaaagcctcaggggtgtgccagacagccctcaacgacgggaaaagctacacaaggtggctccaaaaatcaaagccctgatgatggagtcaggtacgaccatggttggctaccagccccaaggggacaaggccaacttcttccggatggtcatctccaacccagccgctacccagtctgacattgacttcctcattgaggagatagaaagactgggccaggatctgtaatcatccttcgcagaacatgagtttatgggaatgccttttccctctggcactccagaacaaacctctatatgttgctgaaacacacaggccatttcattgagggaaaacataatatcttgaagaatattgttaaaaccttacttaaagcttgtttgttctagttagcaggaaatagtgttctttttaaaaagttgcacattaggaacagagtatatatgtacagttatacatacctctctctatatatacatgtatagtgagtgtggcttagtaatagatcacggcatgtttcccgctccaagagaattcactttaccttcagcagttaccgaggagctaaacatgctgccaaccagcttgtccaacaactccaggaaaactgtttttcaaaacgccatgtcctaggggccaagggaaatgctgttggtgagaatcgacctcactgtcagcgtttctccacctgaagtgatgatggatgagaaaaaacaccaccaaatgacaagtcacaccctccccattagtatcctgttaggggaaaatagtagcagagtcattgttacaggtgtactatggctgtatttttagagattaatttgtgtagattgtgtaaattcctgttgtctgaccttggtggtgggagggggagactatgtgtcatgatttcaatgattgtttaattgtaggtcaatgaaatatttgcttatttatattcagagatgtaccatgttaaagaggcgtcttgtattttcttcccatttgtaatgtatcttatttatatatgaagtaagttctgaaaactgtttatggtattttcgtgcatttgtgagccaaagagaaaagattaaaattagtgagatttgtatttatattagagtgcccttaaaataatgatttaagcattttactgtctgtaagagaattctaagattgtacataaagtcatatatatggaaatcctgttacttaaatagcatctgctcttctcttacgctctctgtctggctgtacgtctggtgttctcaatgcttttctagcaactgttggataataactagatctcctgtaattttgtagtagttgatgaccaatctctgtgactcgcttagctgaaacctaaggcaacatttccgaagaccttctgaagatctcagataaagtgaccaggctcacaactgtttttgaagaagggaaattcacactgtgcgttttagagtatgcaagaagaatataaataaataaaaatattctccatggagaatttgaacaaaaaaaaaaaaaaaa

SEQ ID NO: 202 - protein, 594 aa, Homo sapiens
MASSTPSSSATSSNAGADPNTTNLRPTTYDTWCGVAHGCTRKLGLKICGFLQRTNSLEEKSRLVSAFKERQSSKNLLSCENSDRDARFRRTETDFSNLFARDLLPAKNGEEQTVQFLLEVVDILLNYVRKTFDRSTKVLDFHHPHQLLEGMEGFNLELSDHPESLEQILVDCRDTLKYGVRTGHPRFFNQLSTGLDIIGLAGEWLTSTANTNMFTYEIAPVFVLMEQITLKKMREIVGWSSKDGDGIFSPGGAISNMYSIMAARYKYFPEVKTKGMAAVPKLVLFTSEQSHYSIKKAGAALGFGTDNVILIKCNERGKIIPADFEAKILEAKQKGYVPFYVNATAGTTVYGAFDPIQEIADICEKYNLWLHVDAAWGGGLLMSRKHRHKLNGIERANSVTWNPHKMMGVLLQCSAILVKEKGILQGCNQMCAGYLFQPDKQYDVSYDTGDKAIQCGRHVDIFKFWLMWKAKGTVGFENQINKCLELAEYLYAKIKNREEFEMVFNGEPEHTNVCFWYIPQSLRGVPDSPQRREKLHKVAPKIKALMMESGTTMVGYQPQGDKANFFRMVISNPAATQSDIDFLIEEIERLGQDL

SEQ ID NO: 203 - protein, 21 aa, Artificial sequence
  Gad derived peptide (552-572)
DKVNFFRMVISNPAATHQDID

SEQ ID NO: 204 - protein, 13 aa, Artificial sequence
  HA307-319 peptide
PKYVKQNTLKLAT

SEQ ID NO: 205 - protein, 15 aa, Artificial sequence
  InsA1-15  peptide
GIVEQCCTSICSLYQ

SEQ ID NO: 206 - protein, 13 aa, Artificial sequence
  CII261-273 peptide
AGFKGEQGPKGEP

SEQ ID NO: 207 - protein, 13 aa, Artificial sequence
  GAD65 APL M559Z peptide
NFFRZVISNPAAT

SEQ ID NO: 208 - protein, 13 aa, Artificial sequence
  GAD65 APL I561M peptide
NFFRMVMSNPAAT

SEQ ID NO: 209 - protein, 13 aa, Artificial sequence
  GAD65 APL N563Q peptide
NFFRMVISQPAAT

SEQ ID NO: 210 - protein, 13 aa, Artificial sequence
  GAD65 APL I561M-N563Q peptide
NFFRMVMSQPAAT

SEQ ID NO: 211 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLRKSNAAERRGPL

SEQ ID NO: 212 - protein, 266 aa, Homo sapiens [sequence omitted]

SEQ ID NO: 213 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 214 - protein, 585 aa, Homo sapiens; identical to SEQ ID NO: 200

SEQ ID NO: 215 - protein, 566 aa, Homo sapiens
MPRGFLVKRTKRTGGLYRVRLAERVFPLLGPQGAPPFLEEAPSASLPGAERATPPTREEPGKGLTAEAAREQSGSPCRAAGVSPGTGGREGAEWRAGGREGPGPSPSPSPSPAKPAGAELRRAFLERCLSSPVSAESFPGGAAAVAAFSCSVAPAAAPTPGEQFLLPLRAPFPEPALQPDPAPLSAALQSLKRAAGGERRGKAPTDCASGPAAAGIKKPKAMRKLSFADEVTTSPVLGLKIKEEEPGAPSRGLGGSRTPLGEFICQLCKEQYADPFALAQHRCSRIVRVEYRCPECDKVFSCPANLASHRRWHKPRPAAANAATVSSADGKPPSSSSSSSRDSGAIASFLAEGKENSRIERTADQHPQARDSSGADQHPDSAPRQGLQVLTHPEPPLPQGPYTEGVLGRRVPVPGSTSGGRGSEIFVCPYCHKKFRRQAYLRKHLSTHEAGSARALAPGFGSERGAPLAFACPLCGAHFPTADIREKHRLWHAVREELLLPALAGAPPETSGPSGPSDGSAQQIFSCKHCPSTFFSSPGLTRHINKCHPSESRQVLLLQMPLRPGC

SEQ ID NO: 216 - protein, 355 aa, Homo sapiens
MDFLHRNGVLIIQHLQKDYRAYYTFLNFMSNVGDPRNIFFIYFPLCFQFNQTVGTKMIWVAVIGDWLNLIFKWILFGHRPYWWVQETQIYPNHSSPCLEQFPTTCETGPGSPSGHAMGASCVWYVMVTAALSHTVCGMDKFSITLHRLTWSFLWSVFWLIQISVCISRVFIATHFPHQVILGVIGGMLVAEAFEHTPGIQTASLGTYLKTNLFLFLFAVGFYLLLRVLNIDLLWSVPIAKKWCANPDWIHIDTTPFAGLVRNLGVLFGLGFAINSEMFLLSCRGGNNYTLSFRLLCALTSLTILQLYHFLQIPTHEEHLFYVLSFCKSASIPLTVVAFIPYSVHMLMKQSGKKSQ

SEQ ID NO: 217 - protein, 154 aa, Homo sapiens
MDFLHRNGVLIIQHLQKDYRAYYTFLNFMSNVGDPRNIFFIYFPLCFQFNQTVGTKMIWVAVIGDWLNLIFKWILFGHRPYWWVQETQIYPNHSSPCLEQFPTTCETGPGSPSGHAMGASCVWYVMVTAALSHTVCGMDKFSITLHRHAGGRGL

SEQ ID NO: 218 - protein, 457 aa, Homo sapiens
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVMEKREDSKEAEKSGEATDGARPQALPEPMQESKAEGNNQAPGEEEEEEEEATNTHPPASLPSQKYPGPQAEGDSEGLSQGLVDREKGLSAEPGWQAKREEEEEEEEEAEAGEEAVPEEEGPTVVLNPHPSLGYKEIRKGESRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQLQALRRG

SEQ ID NO: 219 - protein, 369 aa, Homo sapiens
MEFLERTYLVNDKAAKMYAFTLESVELQQKPVNKDQCPRERPEELESGGMYHCHSGSKPTEKGANEYAYAKWKLCSASAICFIFMIAEVVGGHIAGSLAVVTDAAHLLIDLTSFLLSLFSLWLSSKPPSKRLTFGWHRAEILGALLSILCIWVVTGVLVYLACERLLYPDYQIQATVMIIVSSCAVAANIVLTVVLHQRCLGHNHKEVQANASVRAAFVHALGDLFQSISVLISALIIYFKPEYKIADPICTFIFSILVLASTITILKDFSILLMEGVPKSLNYSGVKELILAVDGVLSVHSLHIWSLTMNQVILSAHVATAASRDSQVVRREIAKALSKSFTMHSLTIQMESPVDQDPDCLFCEDPCD

SEQ ID NO: 220 - protein, 573 aa, Homo sapiens
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 221 - protein, 998 aa, Homo sapiens
MGPPLPLLLLLLLLLPPRVLPAAPSSVPRGRQLPGRLDGVFGRCQKVPAMDFYRYEVSPVALQRLRVALQKLSGTGFTWQDDYTQYVMDQELADLPKTYLRRPEASSPARPSKHSVGSERRYSREGGAALANALRRHLPFLEALSQAPASDVLARTHTAQDRPPAEGDDRFSESILTYVAHTSALTYPPGSRTQLREDLLPRTLGQLQPDELSPKVDSGVDRHHLMAALSAYAAQRPPAPPGEGSLEPQYLLRAPSRMPRPLLAPAAPQKWPSPLGDSEDPSSTGDGARIHTLLKDLQRQPAEVRGLSGLELDGMAELMAGLMQGVDHGVARGSPGRAALGESGEQADGPKATLRGDSFPDDGVQDDDDRLYQEVHRLSATLGGLLQDHGSRLLPGALPFARPLDMERKKSEHPESSLSSEEETAGVENVKSQTYSKDLLGQQPHSEPGAAAFGELQNQMPGPSKEEQSLPAGAQEALSDGLQLEVQPSEEEARGYIVTDRDPLRPEEGRRLVEDVARLLQVPSSAFADVEVLGPAVTFKVSANVQNVTTEDVEKATVDNKDKLEETSGLKILQTGVGSKSKLKFLPPQAEQEDSTKFIALTLVSLACILGVLLASGLIYCLRHSSQHRLKEKLSGLGGDPGADATAAYQELCRQRMATRPPDRPEGPHTSRISSVSSQFSDGPIPSPSARSSASSWSEEPVQSNMDISTGHMILSYMEDHLKNKNRLEKEWEALCAYQAEPNSSFVAQREENVPKNRSLAVLTYDHSRVLLKAENSHSHSDYINASPIMDHDPRNPAYIATQGPLPATVADFWQMVWESGCVVIVMLTPLAENGVRQCYHYWPDEGSNLYHIYEVNLVSEHIWCEDFLVRSFYLKNLQTNETRTVTQFHFLSWYDRGVPSSSRSLLDFRRKVNKCYRGRSCPIIVHCSDGAGRSGTYVLIDMVLNKMAKGAKEIDIAATLEHLRDQRPGMVQTKEQFEFALTAVAEEVNAILKALPQ

SEQ ID NO: 222 - protein, 1015 aa, Homo sapiens
MGPPLPLLLLLLLLLPPRVLPAAPSSVPRGRQLPGRLGCLLEEGLCGASEACVNDGVFGRCQKVPAMDFYRYEVSPVALQRLRVALQKLSGTGFTWQDDYTQYVMDQELADLPKTYLRRPEASSPARPSKHSVGSERRYSREGGAALANALRRHLPFLEALSQAPASDVLARTHTAQDRPPAEGDDRFSESILTYVAHTSALTYPPGSRTQLREDLLPRTLGQLQPDELSPKVDSGVDRHHLMAALSAYAAQRPPAPPGEGSLEPQYLLRAPSRMPRPLLAPAAPQKWPSPLGDSEDPSSTGDGARIHTLLKDLQRQPAEVRGLSGLELDGMAELMAGLMQGVDHGVARGSPGRAALGESGEQADGPKATLRGDSFPDDGVQDDDDRLYQEVHRLSATLGGLLQDHGSRLLPGALPFARPLDMERKKSEHPESSLSSEEETAGVENVKSQTYSKDLLGQQPHSEPGAAAFGELQNQMPGPSKEEQSLPAGAQEALSDGLQLEVQPSEEEARGYIVTDRDPLRPEEGRRLVEDVARLLQVPSSAFADVEVLGPAVTFKVSANVQNVTTEDVEKATVDNKDKLEETSGLKILQTGVGSKSKLKFLPPQAEQEDSTKFIALTLVSLACILGVLLASGLIYCLRHSSQHRLKEKLSGLGGDPGADATAAYQELCRQRMATRPPDRPEGPHTSRISSVSSQFSDGPIPSPSARSSASSWSEEPVQSNMDISTGHMILSYMEDHLKNKNRLEKEWEALCAYQAEPNSSFVAQREENVPKNRSLAVLTYDHSRVLLKAENSHSHSDYINASPIMDHDPRNPAYIATQGPLPATVADFWQMVWESGCVVIVMLTPLAENGVRQCYHYWPDEGSNLYHIYEVNLVSEHIWCEDFLVRSFYLKNLQTNETRTVTQFHFLSWYDRGVPSSSRSLLDFRRKVNKCYRGRSCPIIVHCSDGAGRSGTYVLIDMVLNKMAKGAKEIDIAATLEHLRDQRPGMVQTKEQFEFALTAVAEEVNAILKALPQ

SEQ ID NO: 223 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 224 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 225 - protein, 238 aa, Aequorea macrodactyla
MSKGEELFTGIVPVLIELDGDVHGHKFSVRGEGEGDADYGKLEIKFICTTGKLPVPWPTLVTTLGYGIQCFARYPEHMKMNDFFKSAMPEGYIQERTIFFQDDGKYKTRGEVKFEGDTLVNRIELKGMDFKEDGNILGHKLEYNFNSHNVYIMPDKANNGLKVNFKIRHNIEGGGVQLADHYQTNVPLGDGPVLIPINHYLSLQTAISKDRNETRDHMVFLEFFSACGHTHGMDELYK

SEQ ID NO: 226 - DNA, 717 bp, Aequorea macrodactyla
atgagtaaaggagaagaacttttcactgggattgtcccagttctcattgagttagacggtgatgtccatggacataaattctctgtcagaggagaaggggaaggcgatgcagattatggaaaacttgaaatcaaattcatttgcactactggaaagctaccagttccatggccaacacttgttactacactgggctacggcatccaatgtttcgcaagatacccagaacacatgaaaatgaatgacttcttcaagagtgccatgcctgagggttacattcaagaaagaaccatctttttccaagatgatggaaaatacaagacacgtggtgaagtcaagtttgaaggtgatactcttgttaacagaattgagctcaaaggtatggactttaaagaagatggcaatatccttggacacaagttggagtacaattttaattcacataatgtatacattatgccggacaaagccaataatggactcaaagtcaatttcaaaattagacacaatatcgaaggtggtggtgtccaacttgctgatcattaccaaacaaatgttccccttggagacggtcctgtccttataccaatcaatcactacctatccttgcaaacagccatttcaaaagatcgaaatgagacgagagatcatatggtgtttctggaatttttctcagcttgtggacatacacatggcatggatgaactatacaaataa

SEQ ID NO: 227 - protein, 489 aa, Artificial sequence
  Alkaline phosphatase
MKQSTIALALLPLLFTPVTKARTPEMPLQGTAVDGGGGSMHASLEVLENRAAQGDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLK

SEQ ID NO: 228 - DNA, 1470 bp, Artificial sequence
  Alkaline phosphatase coding sequence
ttatttcagccccagagcggctttcatggtgtagaagagatcggtctggtcggtcagtccaacaacattggcggcatgcgggccatacgccgcaatacgcaactgactgccggtatgttcttgtgaatcctcttcggagttcccgtaactcatcaccatcactgcgccatctttggtatttagcgcctgggtgaggcccggagctttggtatccggcgcaacaatctggctggcgtgggcgtgatcagcggtgactatgaccagcgtgttaccctcctttttagcgaattccagcgcccgttgtacggcttcatcgagatcgaccgtctcgccaatttgcccacaaggattcgcagcatgatcctgtttatcgattgacgcaccttcaacttgcaggaaaaagcctttctcatttttactcaacaattcaatggctttgtcggtcatctgcgccagggttggtacactgtcattacgttgcggatttggcgtacaggtgactgcgggcttatcgatattgccatggtacgttgctttcggtcctagccagcgcactggcatattgccgtcagcaaacaggccaagcaggggtttttgctgattcgcttccgtcaccgaattcagtgaggcagcatcgctcaccaactgataaccacgcgcctgtgcctgttcacgcagcgtttttccctgccattcaccagcggttgccgtttcagcaaaggtttttgcgccgccgccaagcgtaacgtcggcacgagcgttaagcagctgttcggtaatcgatccttttccgcctttttccagagcgttacccggacatttttcactggtcgcgctcggaccgtagcatttgcgcgaggtcacatgtgccaccagcgcagcgggcgtggcatcctgcaactctgcggtagaaacgttaccggtcgccagacctgcggcttttgccatttccagaatcgttgggtgatctttttcgtgaatatcgacgcccagcgcgccgttataggttttgacaccggttgaccaggcggttgctgatgcagccgagtcggtgacgtagtccggtttgccggtttttttattcagcgcatagtgagtgtattgcccggtaagcggtaaggcatctatacctttaaaaaagccgcccgcaccttcggcataattacgtgcggcagtaatttccgagtcccccatcccatcgccaatcagcaaaataatattttttgcaggtttatcgctaagagaatcacgcagagcggcagtctgatcacccgttaaacggcgagcaccgccgggtgcagtaatatcgccctgagcagcccggttttccagaacctcgaggctagcatgcatagaaccgccaccaccgtcgacagcggtaccctgcagaggcatttctggtgtccgggcttttgtcacaggggtaaacagtaacggtaagagtgccagtgcaatagtgctttgtttcac

SEQ ID NO: 229 - protein, 308 aa, Artificial sequence
  Horseradish peroxidase
MQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSN

SEQ ID NO: 230 - DNA, 930 bp, Artificial sequence
  Horseradish peroxidase coding sequence
atgcagttaacccctacattctacgacaatagctgtcccaacgtgtccaacatcgttcgcgacacaatcgtcaacgagctcagatccgatcccaggatcgctgcttcaatattacgtctgcacttccatgactgcttcgtgaatggttgcgacgctagcatattactggacaacaccaccagtttccgcactgaaaaggatgcattcgggaacgctaacagcgccaggggctttccagtgatcgatcgcatgaaggctgccgttgagtcagcatgcccacgaacagtcagttgtgcagacctgctgactatagctgcgcaacagagcgtgactcttgcaggcggaccgtcctggagagtgccgctcggtcgacgtgactccctacaggcattcctagatctggccaacgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggactttgatctgcggaccccaaccatcttcgataacaagtactatgtgaatctagaggagcagaaaggcctgatacagagtgatcaagaactgtttagcagtccaaacgccactgacaccatcccactggtgagaagttttgctaactctactcaaaccttctttaacgccttcgtggaagccatggaccgtatgggtaacattacccctctgacgggtacccaaggccagattcgtctgaactgcagagtggtcaacagcaactcttaa

SEQ ID NO: 231 - protein, 286 aa, Artificial sequence
  Synthetic construct fused to 6XHis and C-myc tags
QSVLTQPPSVSGSPGQSITISCTGTSSDIGTYKIVSWYQQHPGKAPKLMIYDVNQRPSGVSDRFSGSKSGNTASLTISGLQAEDEADYYCSSYTSGSTLVFGGGTKLTVLGSSGGGGSGGGGSGGSALQVQLQQSGAEVKKPGESLKISCKGSGYSFTSYWIGWVRQMPGKGLEWMGIIYPGDSDTRYSPSFQGQVTISADKSISTAYLQWSSLKASDTAMYYCARHRAASGSPDACDYWGQGTLVTVSSGSASAPTLFPAAAHHHHHHGAAEQKLISEEDLNGAA

SEQ ID NO: 232 - DNA, 861 bp, Artificial sequence
  Synthetic construct fused to 6XHis and C-myc tags
cagtctgtgttgacgcagccgccctcagtgtctgggtctcctggacagtcgatcaccatctcctgcactggaaccagcagtgatattgggacttataaaattgtctcctggtaccaacagcaccctggcaaagcccccaaactcatgatttatgacgtcaatcagcggccctcaggggtttctgatcgcttctctggctccaagtctggcaacacggcctccctgacaatctctgggctccaggctgaggacgaggctgattattactgcagctcatatacaagcggcagcactctggtattcggcggggggaccaagctgaccgtcctaggctcgagtggtggaggcggttcaggcggaggtggctctggcggtagtgcacttcaggtacagctgcagcagtcaggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacatcgggccgctagtgggagcccggacgcgtgtgactactggggccagggaaccctggtcaccgtctcctcagggagtgcatccgccccaacccttttccccgcggccgcacatcatcatcaccatcacggggccgcagaacaaaaactcatctcagaagaggatctgaatggggccgcatag

SEQ ID NO: 233 - protein, 345 aa, Artificial sequence
  PE38KDEL amino acid sequence
EGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 234 - DNA, 1035 bp, Artificial sequence
  PE38KDEL coding sequence
gagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgaaagacgagctc

SEQ ID NO: 235 - protein, 238 aa, Artificial sequence
  Orange fluorescent protein
MSKGEELFTGVVPILVELDGDVHGHKFSVRGEGEGDADYGKLEIKFICTTGKLPVPWPTLVTTLGYGILCFARYPEHMKMNDFFKSAMPEGYIQERTIFFQDDGKYKTRGEVKFEGDTLVNRIELKGMDFKEDGNILGHKLEYNFNSHNVYIMPDKANNGLKVNFKIRHNIEGGGVQLADHYQTNVPLGDGPVLIPINHYLSYQTAISKDRNETRDHMVFLEFFSACGHTHGMDELYK

SEQ ID NO: 236 - DNA, 717 bp, Artificial sequence
  Orange fluorescent protein coding sequence
atgagtaaaggagaagaacttttcactggagttgtcccaattcttgttgaattagatggtgatgtccatggacataaattctctgtcagaggagaaggggaaggcgatgcagattatggaaaacttgaaatcaaattcatttgcactactggaaagctaccagttccatggccaacacttgttactacactgggctatggcatcctatgtttcgcaagatacccagaacacatgaaaatgaatgacttcttcaagagtgccatgcctgagggttacattcaagaaagaaccatctttttccaagatgatggaaaatacaagacacgtggtgaagtcaagtttgaaggtgatactcttgttaacagaattgagctcaaaggtatggactttaaagaagatggcaatatccttggacacaagttggagtacaattttaactcacataatgtatacattatgccggacaaagccaataatggactcaaagtcaatttcaaaattagacacaatatcgaaggtggtggtgtccaactcgctgatcattaccaaacaaatgttccccttggagacggtcctgtccttataccaatcaatcactacctatcctatcaaacagccatttcaaaagatcgaaatgagacgagagatcatatggtgtttctggaatttttctcagcttgtggacatacacatggcatggatgaactatacaaataa

SEQ ID NO: 237 - protein, 1019 aa, Artificial sequence
  Beta-galactosidase
MADPVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSADFQLSAGRYHYQLVWCQK

SEQ ID NO: 238 - DNA, 3060 bp, Artificial sequence
  Beta-galactosidase coding sequence
ttatttttgacaccagaccaactggtaatggtagcgaccggcgctcagctggaaatccgccgatactgacgggctccaggagtcgtcgccaccaatccccatatggaaaccgtcgatattcagccatgtgccttcttccgcgtgcagcagatggcgatggctggtttccatcagttgctgttgactgtagcggctgatgttgaactggaagtcgccgcgccactggtgtgggccataattcaattcgcgcgtcccgcagcgcagaccgttttcgctcgggaagacgtacggggtatacatgtctgacaatggcagatcccagcggtcaaaacaggcggcagtaaggcggtcgggatagttttcttgcggccctaatccgagccagtttacccgctctgctacctgcgccagctggcagttcaggccaatccgcgccggatgcggtgtatcgctcgccacttcaacatcaacggtaatcgccatttgaccactaccatcaatccggtaggttttccggctgataaataaggttttcccctgatgctgccacgcgtgagcggtcgtaatcagcaccgcatcagcaagtgtatctgccgtgcactgcaacaacgctgcttcggcctggtaatggcccgccgccttccagcgttcgacccaggcgttagggtcaatgcgggtcgcttcacttacgccaatgtcgttatccagcggtgcacgggtgaactgatcgcgcagcggcgtcagcagttgttttttatcgccaatccacatctgtgaaagaaagcctgactggcggttaaattgccaacgcttattacccagctcgatgcaaaaatccatttcgctggtggtcagatgcgggatggcgtgggacgcggcggggagcgtcacactgaggttttccgccagacgccactgctgccaggcgctgatgtgcccggcttctgaccatgcggtcgcgttcggttgcactacgcgtactgtgagccagagttgcccggcgctctccggctgcggtagttcaggcagttcaatcaactgtttaccttgtggagcgacatccagaggcacttcaccgcttgccagcggcttaccatccagcgccaccatccagtgcaggagctcgttatcgctatgacggaacaggtattcgctggtcacttcgatggtttgcccggataaacggaactggaaaaactgctgctggtgttttgcttccgtcagcgctggatgcggcgtgcggtcggcaaagaccagaccgttcatacagaactggcgatcgttcggcgtatcgccaaaatcaccgccgtaagccgaccacgggttgccgttttcatcatatttaatcagcgactgatccacccagtcccagacgaagccgccctgtaaacggggatactgacgaaacgcctgccagtatttagcgaaaccgccaagactgttacccatcgcgtgggcgtattcgcaaaggatcagcgggcgcgtctctccaggtagcgaaagccattttttgatggaccatttcggcacagccgggaagggctggtcttcatccacgcgcgcgtacatcgggcaaataatatcggtggccgtggtgtcggctccgccgccttcatactgcaccgggcgggaaggatcgacagatttgatccagcgatacagcgcgtcgtgattagcgccgtggcctgattcattccccagcgaccagatgatcacactcgggtgattacgatcgcgctgcaccattcgcgttacgcgttcgctcatcgccggtagccagcgcggatcatcggtcagacgattcattggcaccatgccgtgggtttcaatattggcttcatccaccacatacaggccgtagcggtcgcacagcgtgtaccacagcggatggttcggataatgcgaacagcgcacggcgttaaagttgttctgcttcatcagcaggatatcctgcaccatcgtctgctcatccatgacctgaccatgcagaggatgatgctcgtgacggttaacgcctcgaatcagcaacggcttgccgttcagcagcagcagaccattttcaatccgcacctcgcggaaaccgacatcgcaggcttctgcttcaatcagcgtgccgtcggcggtgtgcagttcaaccaccgcacgatagagattcgggatttcggcgctccacagtttcgggttttcgacgttcagacgtagtgtgacgcgatcggcataaccaccacgctcatcgataatttcaccgccgaaaggcgcggtgccgctggcgacctgcgtttcaccctgccataaagaaactgttacccgtaggtagtcacgcaactcgccgcacatctgaacttcagcctccagtacagcgcggctgaaatcatcattaaagcgagtggcaacatggaaatcgctgatttgtgtagtcggtttatgcagcaacgagacgtcacggaaaatgccgctcatccgccacatatcctgatcttccagataactgccgtcactccaacgcagcaccatcaccgcgaggcggttttctccggcgcgtaaaaatgcgctcaggtcaaattcagacggcaaacgactgtcctggccgtaaccgacccagcgcccgttgcaccacagatgaaacgccgagttaacgccatcaaaaataattcgcgtctggccttcctgtagccagctttcatcaacattaaatgtgagcgagtaacaacccgtcggattctccgtgggaacaaacggcggattgaccgtaatgggataggttacgttggtgtagatgggcgcatcgtaaccgtgcatctgccagtttgaggggacgacgacagtatcggcctcaggaagatcgcactccagccagctttccggcaccgcttctggtgccggaaaccaggcaaagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacgggatcagccat

SEQ ID NO: 239 - protein, 159 aa, Artificial sequence
  Streptavidin
DPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 240 - DNA, 480 bp, Artificial sequence
  Streptavidin coding sequence
gacccgagcaaagattctaaagcacaagtatctgctgcagaagcaggaattacaggcacatggtataatcagctgggatctacatttattgttacagccggcgcagatggagctcttacaggaacatatgaatctgctgttggaaatgcagaatctagatacgtgcttacaggaagatatgattctgcacctgcaacagatggatccggaacagcacttggatggacagttgcatggaaaaacaattatagaaacgcacatagcgctacaacatggtctggccaatatgtgggaggtgcagaagcaagaattaacacacaatggcttttaacatctggaacaacagaagcaaatgcatggaaaagtactcttgttggacatgatacatttacaaaagttaaacctagcgcagcatctatcgatgcagcgaaaaaagcaggagttaacaatggcaatcctttagatgcagttcaacaataa

SEQ ID NO: 241 - protein, 216 aa, Pseudomonas aeruginosa
AEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLAPSSIPDQEQAISALPDYASQPGKPSREDLK

SEQ ID NO: 242 - DNA, 651 bp, Pseudomonas aeruginosa
gcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggaccttgacgcgatctggcgcggtttctatatcgccggcgatccggcgctggcctacggctacgcccaggaccaggaacccgacgcgcgcggccggatccgcaacggtgccctgctgcgggtctatgtgccgcgctcgagtctgccgggcttctaccgcaccggcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccacgcaacgtcggcggcgacctcgccccgtccagcatccccgaccaggaacaggcgatcagcgccctgccggactacgccagccagcccggcaaaccgtcgcgcgaggacctgaagtaa

SEQ ID NO: 243 - protein, 536 aa, Corynebacterium diphtheriae
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 244 - DNA, 1611 bp, Corynebacterium diphtheriae
atgggcgctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatcagtaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcatctttaatggttgctcaagctattccattggtaggagagctagttgatattggtttcgctgcatataattttgtagagagtattatcaatttatttcaagtagttcataattcgtataatcgtcccgcgtattctccggggcataaaacgcaaccatttcttcatgacgggtatgctgtcagttggaacactgttgaagattcgataatccgaactggttttcaaggggagagtgggcacgacataaaaattactgctgaaaataccccgcttccaatcgcgggtgtcctactaccgactattcctggaaagctggacgttaataagtccaagactcatatttccgtaaatggtcggaaaataaggatgcgttgcagagctatagacggtgatgtaactttttgtcgccctaaatctcctgtttatgttggtaatggtgtgcatgcgaatcttcacgtggcatttcacagaagcagctcggagaaaattcattctaatgaaatttcatcggattccataggcgttcttgggtaccagaaaacagtagatcacaccaaggttaattctaagctatcgctattttttgaaatcaaaagctga

SEQ ID NO: 245 - protein, 127 aa, Artificial sequence
  Interleukin 2 (IL-2)
TKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 246 - DNA, 384 bp, Artificial sequence
  Interleukin 2 (IL-2)
acaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 247 - protein, 207 aa, Artificial sequence
  Human derived CD3 polypeptide
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 248 - DNA, 624 bp, Artificial sequence
  Human derived CD3 polynucleotide
atgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctga

SEQ ID NO: 249 - protein, 290 aa, Artificial sequence
  CD16
MGGGAGERLFTSSCLVGLVPLGLRISLVTCPLQCGIMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 250 - DNA, 873 bp, Artificial sequence
  CD16 coding region
atgggtggaggggctggggaaaggctgtttacttcctcctgtctagtcggtttggtccctttagggctccggatatctttggtgacttgtccactccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 251 - protein, 153 aa, Artificial sequence [sequence omitted]

SEQ ID NO: 252 - DNA, 462 bp, Artificial sequence
  Interleukin 4 coding sequence
atgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctga

SEQ ID NO: 253 - protein, 365 aa, Artificial sequence [sequence omitted]

SEQ ID NO: 254 - DNA, 1098 bp, Artificial sequence
  Human derived HLA-A2 coding sequence
atggccgtcatggcgccccgaaccctcgtcctgctactctcgggggctctggccctgacccagacctgggcgggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgtga

SEQ ID NO: 255 - protein, 178 aa, Artificial sequence
  Human derived interleukine 10
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 256 - DNA, 537 bp, Artificial sequence
  Human derived interleukine 10 coding sequence
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 257 - protein, 576 aa, Artificial sequence
  Ricin toxin
MKPGGNTIVIWMYAVATWLCFGSTSGWSFTLEDNNIFPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCAPPPSSQFSLLIRPVVPNFNADVCMDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTNNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRASDPSLKQIILYPLHGDPNQIWLPLF

SEQ ID NO: 258 - DNA, 1731 bp, Artificial sequence
  Ricin toxin coding sequence
atgaaaccgggaggaaatactattgtaatatggatgtatgcagtggcaacatggctttgttttggatccacctcagggtggtctttcacattagaggataacaacatattccccaaacaatacccaattataaactttaccacagcgggtgccactgtgcaaagctacacaaactttatcagagctgttcgcggtcgtttaacaactggagctgatgtgagacatgaaataccagtgttgccaaacagagttggtttgcctataaaccaacggtttattttagttgaactctcaaatcatgcagagctttctgttacattagcgctggatgtcaccaatgcatatgtggtcggctaccgtgctggaaatagcgcatatttctttcatcctgacaatcaggaagatgcagaagcaatcactcatcttttcactgatgttcaaaatcgatatacattcgcctttggtggtaattatgatagacttgaacaacttgctggtaatctgagagaaaatatcgagttgggaaatggtccactagaggaggctatctcagcgctttattattacagtactggtggcactcagcttccaactctggctcgttcctttataatttgcatccaaatgatttcagaagcagcaagattccaatatattgagggagaaatgcgcacgagaattaggtacaaccggagatctgcaccagatcctagcgtaattacacttgagaatagttgggggagactttccactgcaattcaagagtctaaccaaggagcctttgctagtccaattcaactgcaaagacgtaatggttccaaattcagtgtgtacgatgtgagtatattaatccctatcatagctctcatggtgtatagatgcgcacctccaccatcgtcacagttttctttgcttataaggccagtggtaccaaattttaatgctgatgtttgtatggatcctgagcccatagtgcgtatcgtaggtcgaaatggtctatgtgttgatgttagggatggaagattccacaacggaaacgcaatacagttgtggccatgcaagtctaatacagatgcaaatcagctctggactttgaaaagagacaatactattcgatctaatggaaagtgtttaactacttacgggtacagtccgggagtctatgtgatgatctatgattgcaatactgctgcaactgatgccacccgctggcaaatatgggataatggaaccatcataaatcccagatctagtctagttttagcagcgacatcagggaacagtggtaccacacttacagtgcaaaccaacatttatgccgttagtcaaggttggcttcctactaataatacacaaccttttgtgacaaccattgttgggctatatggtctgtgcttgcaagcaaatagtggacaagtatggatagaggactgtagcagtgaaaaggctgaacaacagtgggctctttatgcagatggttcaatacgtcctcagcaaaaccgagataattgccttacaagtgattctaatatacgggaaacagttgtcaagatcctctcttgtggccctgcatcctctggccaacgatggatgttcaagaatgatggaaccattttaaatttgtatagtgggttggtgttagatgtgagggcatcggatccgagccttaaacaaatcattctttaccctctccatggtgacccaaaccaaatatggttaccattattttga

SEQ ID NO: 259 - protein, 15 aa, Artificial sequence
  Biotin lygase tag
LHHILDAQKMVWNHR

SEQ ID NO: 260 - protein, 10 aa, Artificial sequence
  GAD556-565 peptide
FFRMVISNPA

SEQ ID NO: 261 - protein, 30 aa, Artificial sequence
  Exemplary linker peptide
GGGGSGGGGSGGGGSGGGGSGGGGSGGGGS

SEQ ID NO: 262 - protein, 15 aa, Artificial sequence
  Exemplary linker peptide
GGGSLVPRGSGGGGS

SEQ ID NO: 263 - protein, 16 aa, Artificial sequence
  Exemplary linker peptide
GGGGSLVPRGSGGGGS

SEQ ID NO: 264 - protein, 198 aa, Homo sapiens
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 265 - DNA, 594 bp, Homo sapiens
ggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgagtctatcctgaggtgactgtgtatcctgcaaagacccagcccctgcagcaccacaacctcctggtctgctctgtgaatggtttctatccaggcagcattgaagtcaggtggttccggaacggccaggaagagaagactggggtggtgtccacaggcctgatccagaatggagactggaccttccagaccctggtgatgctggaaacagttcctcggagtggagaggtttacacctgccaagtggagcacccaagcctgacgagccctctcacagtggaatggagagcacggtctgaatctgcacagagcaag

SEQ ID NO: 266 - DNA, 39 bp, Artificial sequence
  GAD peptide coding polynucleotide sequence
aacttctttcgtatggttatcagcaatccagctgcgact

SEQ ID NO: 267 - protein, 17 aa, Artificial sequence
  Diabetes-associated autoantigenic peptide
VNFFRMVISNPAATHQD

SEQ ID NO: 268 - protein, 21 aa, Artificial sequence
  Diabetes-associated autoantigenic peptide
DKVNFFRMVISNPAATHQDID

SEQ ID NO: 269 - protein, 8 aa, Artificial sequence
  GAD556-563 peptide
FFRMVISN

SEQ ID NO: 270 - protein, 986 aa, Homo sapiens
MGPPLPLLLLLLLLLPPRVLPAAPSSVPRGRQLPGRLGCLLEEGLCGASEACVNDGVFGRCQKVPAMDFYRYEVSPVALQRLRVALQKLSGTGFTWQDDYTQYVMDQELADLPKTYLRRPEASSPARPSKHSVGSERRYSREGGAALANALRRHLPFLEALSQAPASDVLARTHTAQDRPPAEGDDRFSESILTYVAHTSALTYPPGSRTQLREDLLPRTLGQLQPDELSPKVDSGVDRHHLMAALSAYAAQRPPAPPGEGSLEPQYLLRAPSRMPRPLLAPAAPQKWPSPLGDSEDPSSTGDGARIHTLLKDLQRQPAEVRGLSGLELDGMAELMAGLMQGVDHGVARGSPGRAALGESGEQADGPKATLRGDSFPDDGVQDDDDRLYQEVHRLSATLGGLLQDHGSRLLPGALPFARPLDMERKKSEHPESSLSSEEETAGVENVKSQTYSKDLLGQQPHSEPGAAAFGELQNQMPGPSKEEQSLPAGAQEALSDGLQLEVQPSEEEARGYIVTDREVLGPAVTFKVSANVQNVTTEDVEKATVDNKDKLEETSGLKILQTGVGSKSKLKFLPPQAEQEDSTKFIALTLVSLACILGVLLASGLIYCLRHSSQHRLKEKLSGLGGDPGADATAAYQELCRQRMATRPPDRPEGPHTSRISSVSSQFSDGPIPSPSARSSASSWSEEPVQSNMDISTGHMILSYMEDHLKNKNRLEKEWEALCAYQAEPNSSFVAQREENVPKNRSLAVLTYDHSRVLLKAENSHSHSDYINASPIMDHDPRNPAYIATQGPLPATVADFWQMVWESGCVVIVMLTPLAENGVRQCYHYWPDEGSNLYHIYEVNLVSEHIWCEDFLVRSFYLKNLQTNETRTVTQFHFLSWYDRGVPSSSRSLLDFRRKVNKCYRGRSCPIIVHCSDGAGRSGTYVLIDMVLNKMAKGAKEIDIAATLEHLRDQRPGMVQTKEQFEFALTAVAEEVNAILKALPQ

SEQ ID NO: 271 - protein, 641 aa, Homo sapiens; identical to SEQ ID NO: 224

SEQ ID NO: 272 - protein, 60 aa, Artificial sequence
  Exemplary linker peptide
GSGSGSGSGSGSGSGSGSGSGSGSGSGSGSGSGSGSGSGSGSGSGSGSGSGSGSGSGSGS

(19) United States
(12) Patent Application Publication
(10) Pub. No.: US 2013/0189284 A1
(43) Pub. Date: Jul. 25, 2013

(54) ANTIBODIES WITH T-CELL RECEPTOR LIKE SPECIFICITY TOWARDS NATIVE COMPLEXES OF MHC CLASS II AND DIABETES-ASSOCIATED AUTOANTIGENIC PEPTIDES

(71) Applicants: Yoram Reiter, Haifa (IL); Rony Dahan, Mazkeret Batia (IL)

(72) Inventors: Yoram Reiter, Haifa (IL); Rony Dahan, Mazkeret Batia (IL)

(73) Assignee: Technion Research & Development Foundation Ltd., Haifa, IL (US)

(21) Appl. No.: 13/810,248

(22) PCT Filed: Jul. 14, 2011
(86) PCT No.: PCT/IL2011/000563

§ 371 Date: Jan. 15, 2013

Related U.S. Application Data
(60) Provisional application No. 61/364,437, filed on Jul. 15, 2010.

(51) Int. Cl. C07K 14/705; C07K 14/62; C12N 9/16; G01N 33/68; C12N 9/88; C07K 16/28; C07K 14/47

(52) U.S. Cl. CPC C07K 14/705; C07K 16/28; C07K 14/62; C07K 14/47; G01N 33/6854; G01N 33/6893; C12N 9/88; C12N 9/16
USPC 424/173.1; 530/395; 530/389.6; 536/23.4; 424/192.1; 530/391.3; 506/9; 435/7.24; 530/391.7

(57) ABSTRACT

Provided are isolated complexes comprising a major histocompatibility complex (MHC) class II and a type I diabetes-associated autoantigenic peptide, the isolated complex having a structural conformation which enables isolation of a high affinity entity which comprises an antigen binding domain capable of specifically binding to a native conformation of a complex composed of the MHC class II and the type I diabetes-associated autoantigenic peptide; and isolated high affinity entities comprising an antigen binding domain capable of specifically binding the complex, wherein the isolated high affinity entity does not bind to the MHC class II in an absence of the diabetes-associated autoantigenic peptide, wherein the isolated high affinity entity does not bind to the diabetes-associated autoantigenic peptide in an absence of the MHC class II; and methods and kits using same for diagnostic and therapeutic purposes.

### FIELD AND BACKGROUND OF THE INVENTION

The present invention, in some embodiments thereof, relates to isolated complexes of MHC class II and diabetes-associated autoantigenic peptides, isolated high affinity entities such as antibodies which specifically bind to same and, more particularly, but not exclusively, to uses thereof for diagnosing and treating type I diabetes.

Major histocompatibility complex (MHC) class II molecules are expressed in professional antigen presenting cells (APCs) such as macrophages, dendritic cells and B cells. Each MHC class II molecule is a heterodimer composed of two homologous subunits, alpha chain (with α1 and α2 domains) and beta chain (with β1 and β2 domains). Peptides, which are derived from extracellular proteins, enter the cells via endocytosis, are digested in the lysosomes and further bind to MHC class II molecules for presentation on the membrane.

Antigen-specific activation or regulation of CD4+T cells is a multistep process in which co-ligation of the T cell receptor (TCR) with complexes of MHC II/peptide on the surface of APCs plays a central role.

MHC class II molecules with bound self peptides presented by professional APCs play a central role in activating specific CD4+ T cells involved in autoimmune diseases such as Type 1 Diabetes (T1D).

T1D (also known as juvenile diabetes) occurs when the autoimmune destruction of pancreatic beta-islet cells prevents production of the hormone insulin. This causes an inability to regulate glucose metabolism, which results in dangerously raised blood glucose concentrations. It is generally accepted that thymus-derived lymphocytes (T cells) are critically involved in the onset and progression of type 1 diabetes, but the antigens that initiate and drive this destructive process remain poorly characterized—although several candidates have been considered such as insulin, insulin derivatives, islet-specific glucose-6-phosphatase catalytic subunit related peptide (IGRP), carboxypeptidase H, insulinoma-associated antigen (IA-2), glutamic acid decarboxylase (GAD65), carboxypeptidase E and heat shock protein 60.

Genetic factors affecting susceptibility to T1D include the Insulin-Dependent Diabetes Mellitus 1 (IDDM1) gene (GeneID 7924) which is located in the MHC class II region on chromosome 6p21 and which is likely to be responsible for the histocompatibility disorder characteristic of type 1 diabetes in which pancreatic beta cells display improper antigens to T cells. Linkage analysis shows that 96% of diabetic patients express HLA-DR3 and/or HLA-DR4, including over-representation of the HLA-DR3/DR4 heterozygosity in diabetics as compared with non-diabetic controls. These alleles are tightly linked to HLA-DQ alleles that confer susceptibility to IDDM. Other non-genetic factors which might affect susceptibility to type 1 diabetes include diet, which affects gut flora, intestinal permeability, and immune function in the gut.

Glutamate decarboxylase (GAD) enzyme in mammals exists in two isoforms-GAD 65 kDa (GAD2; GeneID 2572) and GAD 67 kDa (GAD1; GeneID 2571). While both isoforms are expressed in brain, GAD 65 kDa is also expressed in the pancreas. Importance of GAD as an islet autoantigen initially highlighted because of the high frequency of auto-antibodies in patient sera directed against this molecule. Subsequent studies led to a large accumulation of data, which support the notion that a dominant CD4+ T-cell response to GAD 65 kDa is a relevant marker for cellular autoimmunity in T1D (Nepom G T. 2003. Conversations with GAD. J. Autoimmun.20:195-8).

Based on the high association of the HLA-DR4 gene to T1D, many epitope identification studies were done, revealing a limited number of GAD peptides presented by the DR4 molecule (Nepom, G. T., et al., 2001). Human CD4+ T cell responses to the DR4/GAD peptides were obtained both among T1D patients and controls (Masewicz, S. A., et al., 2002; Bach, J. M. et al., 1997; Ou, D., et al., 1999; Roep, B. O., et al., 1999; Lohmann, T. et al., 1996; Rharbaoui, et al., 1999), suggesting that the potential for autoreactivity is present in many individuals.

GAD₅₅₅₋₅₆₇ peptide in the context of HLA-DR4 has been shown to be an efficiently processed immunodominant epitope in patients with type 1 diabetes and DR401 transgenic mice (Reijonen, H., et al., 2002; Patel, S. D., et al., 1997). DR4/GAD₅₅₅₋₅₆₇ tetramer detection of autoreactive CD4+ T-cells were observed in the peripheral blood of T1D and at risk subjects but not in healthy controls (Oling, V., et al., 2005).

Additional background art includes U.S. Patent Application No. 20020114816 (ENDL, JOSEF; et al.); U.S. Patent Application No. 20090155292; U.S. Patent Application No. 20030166277; and Krogsgaard M., et al., 2000, Journal pf Experimental Medicine, Pages 1395-1412).

### SUMMARY OF THE INVENTION

According to an aspect of some embodiments of the present invention there is provided an isolated complex comprising a major histocompatibility complex (MHC) class II and a type I diabetes-associated autoantigenic peptide, the isolated complex having a structural conformation which enables isolation of a high affinity entity which comprises an antigen binding domain capable of specifically binding to a native conformation of a complex composed of the MHC class II and the type I diabetes-associated autoantigenic peptide.

According to an aspect of some embodiments of the present invention there is provided an isolated high affinity entity comprising an antigen binding domain capable of specifically binding a complex composed of a major histocompatibility complex (MHC) class II and a type I diabetes-associated autoantigenic peptide, wherein the isolated high affinity entity does not bind to the MHC class II in an absence of the diabetes-associated autoantigenic peptide, wherein the isolated high affinity entity does not bind to the diabetes-associated autoantigenic peptide in an absence of the MHC class II.

According to an aspect of some embodiments of the present invention there is provided an isolated high affinity entity comprising an antigen binding domain being isolatable by the complex of some embodiments of the invention.

According to an aspect of some embodiments of the present invention there is provided an isolated high affinity entity comprising an antigen binding domain capable of specifically binding to the isolated complex of some embodiments of the invention.

According to an aspect of some embodiments of the present invention there is provided an isolated high affinity entity comprising complementarity determining regions (CDRs) set forth by SEQ ID NOs:171-173 and 177-179 (CDRs 1-3 of light and heavy chains of G3H8); or SEQ ID NOs:183-185 and 189-191 (CDRs 1-3 of light and heavy chains G1H12).

According to an aspect of some embodiments of the present invention there is provided a method of isolating a high affinity entity which specifically binds to a complex composed of a major histocompatibility complex (MHC) class II and a type I diabetes-associated autoantigenic peptide, comprising:

(a) screening a library comprising a plurality of high affinity entities with the isolated complex of some embodiments of the invention; and

(b) isolating at least one high affinity entity which specifically binds to the isolated complex of some embodiments of the invention and not to the MHC class II in the absence of the type I diabetes-associated autoantigenic peptide or to the type I diabetes-associated autoantigenic peptide in an absence of the MHC class II,

thereby isolating the high affinity entities which specifically bind to the complex of the MHC class II and the type I diabetes-associated autoantigenic peptide.

According to an aspect of some embodiments of the present invention there is provided a molecule comprising the isolated high affinity entity of some embodiments of the invention, being conjugated to a detectable moiety.

According to an aspect of some embodiments of the present invention there is provided an isolated antibody comprising a multivalent form of the antibody or of the antibody fragment of some embodiments of the invention.

According to an aspect of some embodiments of the present invention there is provided a molecule comprising the isolated high affinity entity of some embodiments of the invention, being conjugated to a therapeutic moiety.

According to an aspect of some embodiments of the present invention there is provided a pharmaceutical composition comprising as an active ingredient the isolated high affinity entity of some embodiments of the invention, the molecule of some embodiments of the invention, or the antibody of some embodiments of the invention, and a pharmaceutically acceptable carrier.

According to an aspect of some embodiments of the present invention there is provided a method of detecting presentation of a type I diabetes-associated autoantigenic peptide on a cell, comprising contacting the cell with the high affinity entity of some embodiments of the invention, the molecule of some embodiments of the invention, or the antibody of some embodiments of the invention, under conditions which allow immunocomplex formation, wherein a presence or a level above a predetermined threshold of the immunocomplex is indicative of presentation of the diabetes-associated autoantigenic peptide on the cell.

According to an aspect of some embodiments of the present invention there is provided a method of diagnosing type 1 diabetes (T1D) in a subject, comprising contacting a cell of the subject with the high affinity entity of some embodiments of the invention, the molecule of some embodiments of the invention, or the antibody of some embodiments of the invention under conditions which allow immunocomplex formation, wherein a presence or a level above a pre-determined threshold of the immunocomplex in or on the cell is indicative of the type 1 diabetes in the subject.

According to an aspect of some embodiments of the present invention there is provided a method of treating type 1 diabetes (T1D), comprising administering to a subject in need thereof a therapeutically effective amount of the high affinity entity of some embodiments of the invention, the molecule of some embodiments of the invention, or the antibody of some embodiments of the invention or the pharmaceutical composition of some embodiments of the invention, thereby treating the type 1 diabetes.

According to an aspect of some embodiments of the present invention there is provided a kit for detecting presence and/or level of a complex which comprises major histocompatibility complex (MHC) class II and a type I diabetes-associated autoantigenic peptide, the kit comprising the high affinity entity of some embodiments of the invention, the molecule of some embodiments of the invention, or the antibody of some embodiments of the invention.

According to an aspect of some embodiments of the present invention there is provided an isolated polynucleotide comprising a first nucleic acid sequence encoding an extracellular domain of an MHC class II beta chain and a second nucleic acid sequence encoding a diabetes-associated autoantigenic peptide, wherein the second nucleic acid sequence being translationally fused upstream of the first nucleic acid sequence or between the nucleic acid sequence encoding amino acids 1-6 of the extracellular domain. According to an aspect of some embodiments of the present invention there is provided a nucleic acid system comprising:

(i) a first polynucleotide comprising the isolated polynucleotide of some embodiments of the invention; and

(ii) a second polynucleotide which comprises a forth nucleic acid sequence encoding an MHC class II alpha chain.

According to an aspect of some embodiments of the present invention there is provided a composition of matter comprising the isolated complex of some embodiments of the invention and a functional moiety conjugated thereto.

According to an aspect of some embodiments of the present invention there is provided a pharmaceutical composition comprising the composition of matter of some embodiments of the invention and a therapeutically acceptable carrier.

According to some embodiments of the invention, the high affinity entity does not bind to the MHC class II in an absence of the diabetes-associated autoantigenic peptide, wherein the isolated high affinity entity does not bind to the diabetes-associated autoantigenic peptide in an absence of the MHC class II.

According to some embodiments of the invention, the diabetes-associated autoantigenic peptide is covalently bound at a C terminus thereof to an N-terminus of an extracellular domain of a beta chain of the MHC class II.

According to some embodiments of the invention, the diabetes-associated autoantigenic peptide is covalently embedded between amino acids 1-6 of an extracellular domain of a beta chain of the MHC class II.

According to some embodiments of the invention, the diabetes-associated autoantigenic peptide is flanked at a C-terminus thereof by a linker peptide. According to some embodiments of the invention, wherein diabetes-associated autoantigenic peptide being translationally fused to the extracellular domain.

According to some embodiments of the invention, the beta chain of the MHC class II comprises a first member of a binding pair which upon expression in eukaryotic cells binds to a second member of the binding pair, wherein the second member is comprised in an alpha chain of the MHC class II, wherein the beta chain and the alpha chain form the MHC class II.

According to some embodiments of the invention, the antigen binding domain is capable of specifically binding to a native conformation of the complex composed of the MHC class II and the type I diabetes-associated autoantigenic peptide.

According to some embodiments of the invention, the antigen binding domain of the isolated high affinity entity is capable of specifically binding to a native conformation of a complex composed of the MHC class II and the type I diabetes-associated autoantigenic peptide.

According to some embodiments of the invention, the antigen binding domain of the isolated high affinity entity is further capable of specifically binding to the isolated complex of some embodiments of the invention.

According to some embodiments of the invention, the high affinity entity further specifically binds to a native conformation of the complex of the MHC class II and the type I diabetes-associated autoantigenic peptide.

According to some embodiments of the invention, the native conformation comprises the structural conformation of the complex of the type I diabetes-associated autoantigenic peptide and the MHC class II when presented on an antigen presenting cell (APC).

According to some embodiments of the invention, the high affinity entity is selected from the group consisting of an antibody, an antibody fragment, a phage displaying an antibody, a peptibody, a bacteria displaying an antibody, a yeast displaying an antibody, and a ribosome displaying an antibody.

According to some embodiments of the invention, the high affinity entity is an antibody or an antibody fragment.

According to some embodiments of the invention, the diabetes-associated autoantigenic peptide is derived from a polypeptide selected from the group consisting of preproinsulin (SEQ ID NO:213), proinsulin (SEQ ID NO:223), Glutamic acid decarboxylase (GAD (SEQ ID NO:214), Insulinoma Associated protein 2 (IA-2; SEQ ID NO:215), IA-213 (SEQ ID NO:221), Islet-specific Glucose-6-phosphatase catalytic subunit-Related Protein (IGRP isoform 1 (SEQ ID NO:216), and Islet-specific Glucose-6-phosphatase catalytic subunit-Related Protein (IGRP isoform 2 (SEQ ID NO:217), chromogranin A (ChgA) (SEQ ID NO:218), Zinc Transporter 8 (ZnT8 (SEQ ID NO:219), Heat Shock Protein-60 (HSP-60; SEQ ID NO:220), Heat Shock Protein-70 (HSP-70; SEQ ID NO:271 and 224).

According to some embodiments of the invention, the diabetes-associated autoantigenic peptide comprises the amino acid sequence selected from the group consisting of SEQ ID NOs:1-157 and no more than 30 amino acids in length.

According to some embodiments of the invention, the diabetes-associated autoantigenic peptide is selected from the group consisting of SEQ ID NOs:1-157, 260, and 267-268.

According to some embodiments of the invention, the diabetes-associated autoantigenic peptide is a Glutamic acid decarboxylase (GAD) autoantigenic peptide.

According to some embodiments of the invention, the GAD autoantigenic peptide comprises a core amino acid sequence set forth by SEQ ID NO:260 (GAD556-565, FFRMVISNPA).

According to some embodiments of the invention, the GAD autoantigenic peptide comprises a core amino acid sequence set forth by SEQ ID NO:260 (GAD556-565, FFRMVISNPA) and no more than 30 amino acids.

According to some embodiments of the invention, the GAD autoantigenic peptide is GAD₅₅₅₋₅₆₇ (NFFRMVISNPAAT; SEQ ID NO:12).

According to some embodiments of the invention, the MHC class II is selected from the group consisting of HLA-DM, HLA-DO, HLA-DP, HLA-DQ, and HLA-DR.

According to some embodiments of the invention, the beta chain of the MHC class II is DR-B1*0401.

According to some embodiments of the invention, the alpha chain of the MHC class II is DR-A1*0101.

According to some embodiments of the invention, the antigen binding domain comprises complementarity determining regions (CDRs) set forth by SEQ ID NOs:171-173 and 177-179 (CDRs 1-3 of light and heavy chains of G3H8); or SEQ ID NOs:183-185 and 189-191 (CDRs 1-3 of light and heavy chains G1H12).

According to some embodiments of the invention, the multivalent form is an IgG antibody.

According to some embodiments of the invention, the high affinity entity is capable of blocking presentation of the complex comprising the MHC class II and the type I diabetes-associated autoantigenic peptide on antigen presenting cells.

According to some embodiments of the invention, the high affinity entity is capable of killing antigen presenting cells which display the complex comprising the MHC class II and the type I diabetes-associated autoantigenic peptide.

According to some embodiments of the invention, the kit further comprising instructions for use in diagnosing type 1 diabetes.

According to some embodiments of the invention, the isolated polynucleotide further comprises a nucleic acid sequence encoding a linker peptide being translationally fused downstream of the second nucleic acid sequence.

According to some embodiments of the invention, the isolated polynucleotide further comprises a third nucleic acid sequence encoding a first member of a binding pair which upon expression in eukaryotic cells binds to a second member of the binding pair.

According to some embodiments of the invention, the second polynucleotide further comprises a fifth nucleic acid construct encoding the second member of the binding pair.

According to some embodiments of the invention, the isolated complex does not include a heterologous immunoglobulin attached thereto.

According to some embodiments of the invention, the functional moiety comprises an antibody or a fragment specific for a cell surface marker.

According to some embodiments of the invention, the diabetes-associated autoantigenic peptide is covalently attached to the beta chain between the third and forth amino acids of a mature polypeptide of the MHC class II beta chain.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D depict the production of recombinant DR4/GAD₅₅₅₋₅₆₇ complex. FIG. 1A-A schematic presentation of the DR-A and DR-B constructs for production in S2 cells. FIGS. 1B-C—SDS-PAGE analyses of purified DR4/GAD complex. The DR4 complex is highly purified and forms SDS-stable hetrodimer. Boiling of the sample disassociates the DR-A and DR-B chains (FIG. 1B; “B”—boiled, “NB”—not boiled). High biotinylation levels were verified by incubation of purified DR4-GAD complexes with increasing concentrations of streptavidin prior to SDS-PAGE analysis (FIG. 1C). All detectable DR-A chains were biotinylated and therefore bound to the streptavidin. FIG. 1D—DR4/GAD complex is folded in the right native conformation. ELISA binding assay of immobilized DR4/GAD-555-567 complex with diluted concentrations of anti-DR conformation sensitive mAb (L243) and anti-DR mAb TU39.

FIGS. 2A-E depict characterization of G3H8 and G1H12 TCRL Fabs directed at DR4/GAD₅₅₅₋₅₆₇, FIG. 2A-ELISA of purified TCRL Fabs with immobilized DR4/GAD₅₅₅₋₅₆₇, control complex DR4/HA₃₀₇₋₃₁₉, GAD₅₅₅₋₅₆₇ peptide, and HA₃₀₇₋₃₁₉ peptide. Anti-DR mAb L243 was used to determine the correct conformation and stability of the bound complexes during the binding assay. Note the specific binding of Fab antibodies G1A1, G1A2 and G3H8 (clone G3H8) and G1H12 (clone G1H12) to the DR4/GAD₅₅₅₋₅₆₇ complex as compared to absence of binding to the other control peptide complexes. FIG. 2B-Flow cytometry analysis of Fab G3H8 binding to Preiss APCs pulsed with GAD₅₅₅₋₅₆₇ peptide or the control peptides: InsA₁₋₁₅, CII₂₆₁₋₂₇₃, Ha₃₀₇₋₃₁₉. FIG. 2C—Flow cytometry analysis of Fab G3H8 to the naturally processed peptide GAD₅₅₂₋₅₇₂. FIG. 2D—binding intensity of the Fab G3H8 antibody at various antibody's concentrations (20, 50 and 100 μg/ml). FIG. 2E—binding intensity of the Fab G3H8 to various loaded GAD₅₅₅₋₅₆₇ peptide concentrations (0, 50, 75, 150, 300 and 400 μg/ml). Note that the binding intensity is dose-dependent on antibody's concentration (FIG. 2D) and peptide concentration (FIG. 2E).

FIGS. 3A-F are flow cytometry analyses depicting the mapping of the recognition epitope of DR4/GAD TCRLs. Flow cytometry analysis of Fab G3H8 binding to Preiss APCs pulsed with wild type (WT) GAD₅₅₅₋₅₆₇ peptide (FIG. 3A), GAD altered peptide ligand (APL): M559Z (FIG. 3B), I561M (FIG. 3C), N563Q (FIG. 3D), I561M+N563Q (FIG. 3E), and the control HA307-319 peptide (FIG. 3F).

FIGS. 4A-B are graphs depicting G3H8Fab ability to inhibit DR4-restricted GAD-specific T cell response to GAD₅₅₅₋₅₆₇ peptide. T cell hybridomas were Ag-specific activated by peptide-pulsed DR0401-Tg splenocytes in the presence of increasing Fab concentrations. FIG. 4A—G2.1.38.1 hybridoma specific to the DR4/GAD₅₅₅₋₅₆₇ epitope was inhibited in a dose-depended manner by G3H8Fab and not by control 1F11 TCRL Fab. FIG. 4B—H1.13.2 hybridoma specific to the DR4/Ha₃₀₇₋₃₁₉ epitope was not inhibited by G3H8 TCRL Fab. These results demonstrate that G3H8 can inhibit GAD₅₅₅₋₅₆₇ specific DR0401 restricted T cell hybridoma response.

FIGS. 5A-E are photographs depicting immunofluorescence analysis using G3H8Fab antibody demonstrating GAD₅₅₅₋₅₆₇ presentation by DR4 in islets of Langerhans of diabetic mice. Frozen sections from diabetic B7/0401 (FIGS. 5A-C) and C57BL/6 (FIGS. 5D-E) mice were subjected to immunostaining analysis using the G3H8 antibody followed by staining with an anti-human IgG-Alexa-488 (green) and 4′,6-diamidino-2-phenylindole (DAPI; blue). Sections were visualized by Cell Observer—Zeiss Fluorescent Microscope. Note the green labeling in islets of Langerhans in B7/041 diabetic mice (FIGS. 5A-C) and the absence of labeling in control C57BL/6 mice (FIGS. 5D-E).

FIGS. 6A-D depict the amino acid [FIGS. 6A (SEQ ID NO:158) and 6C (SEQ ID NO:160)] and nucleic acid [FIGS. 6B (SEQ ID NO:159) and 6D (SEQ ID NO:161)] sequence of the G3H8Fab antibody (Anti HLA-DR4/GAD555-567 Fab) light chain (FIGS. 6A-B) and heavy chain (FIGS. 6C-D). CDRs (by Kabat definition) are underlined (SEQ ID NOs:171-173 CDRs 1-3 for light chain; SEQ ID NOs:177-179 CDRs 1-3 for heavy chain; SEQ ID NO:s174-176 nucleic acid sequence encoding CDRs 1-3 of light chain; SEQ ID NOs:180-182 nucleic acid sequence encoding CDRs 1-3 of heavy chain). For heavy chains: Black letter—VH (variable domain) Blue letters—constant 1 domain (CH1); Red letters—Connector; Purple letters—His tag; Green letters—Myc tag.

FIGS. 7A-D depict the amino acid [FIGS. 7A (SEQ ID NO:162) and 7C (SEQ ID NO:164)] and the nucleic acid [FIGS. 7B (SEQ ID NO:163) and 7D (SEQ ID NO:165)] sequence of the G1H12 (Anti HLA-DR4/GAD555-567 Fab) antibody light chain (FIGS. 7A-B) and heavy chain (FIGS. 7C-D). CDRs (by Kabat definition) are underlined (SEQ ID NOs:183-185 CDRs 1-3 for light chain; SEQ ID NOs:189-191 CDRs 1-3 for heavy chain; SEQ ID NO:s186-188 nucleic acid sequence encoding CDRs 1-3 of light chain; SEQ ID NOs:192-194 nucleic acid sequence encoding CDRs 1-3 of heavy chain). For heavy chains: Black letter—VH (variable domain) Blue letters—CH1 (constant 1 domain); Red letters—Connector; Purple letters—His tag; Green letters—Myc tag.

FIGS. 8A-B depict the amino acid sequence of the recombinant beta (DRB1*0401; FIG. 8A) and alpha (DRA1*0101; FIG. 8B) chains according to some embodiments of the invention. FIG. 8A—leader peptide—highlighted in yellow, beta chain (red), GAD-555-567 peptide (blue), linker (black and underlined), Jun dimerization domain (Green); FIG. 8B-leader peptide—highlighted in yellow, alpha chain (red), GAD-555-567 peptide (blue), linker (black and underlined), Jun dimerization domain (Green) BirA tag (purple).

FIGS. 9A-B depict the nucleic acid sequence of the recombinant beta (DRB1*0401; FIG. 9A) and alpha (DRA1*0101; FIG. 9B) chains according to some embodiments of the invention. FIG. 9A—leader peptide—highlighted in yellow, beta chain (red), GAD-555-567 peptide (blue), linker (black and underlined), Jun dimerization domain (Green); FIG. 9B-leader peptide—highlighted in yellow, alpha chain (red), GAD-555-567 peptide (blue), linker (black and underlined), Jun dimerization domain (Green) BirA tag (purple).

FIGS. 10A-B are histograms depicting flow cytometry analyses depicting binding of G3H8 to murine lymph node cells. Flow cytometry analysis of G3H8 IgG binding to cell suspensions derived from inguinal (draining) lymph nodes (LN) of HLA-DR4 Transgenic (Tg) mice immunized with GAD-555-567 (FIG. 10A) or HA-306-318 (FIG. 10B). Y-axis depicts mean fluorescence intensity of positive cells. X-axis depicts forward side scatter (FCS) counts. Note that while the G3H8 antibody detects APCs presenting the HLA-DR4-GAD-555-567 complexes (6.5% positive cells) from HLA-DR4 Transgenic mice immunized with GAD-555567 (FIG. 10A), this antibody does not detect cells expressing the HLA-DR4—HA-306-318 (background level of 0.9%) from HLA-DR4 Transgenic mice immunized with HA-306-318 (FIG. 10B). Non-draining para-aortic LN and spleen cell suspensions from GAD-immunized mice did not show staining above background levels obtained from the HA-immunized mice (data not shown). These results demonstrate specific detection of GAD-555-567 presenting APCs from inguinal lymph node of GAD-immunized DR4 mice.

FIGS. 11A-C are histograms depicting the increased binding and T-cell blocking capacity of the G3H8 IgG1 antibody compared to that of the G3H8Fab. FIG. 11A—A histogram depicting binding of Fab or IgG G3H8 antibodies to DR4+ Priess cells loaded with the GAD555-567 peptide. Note that the fully human G3H8 IgG1 Ab maintains specificity to DR4/GAD and binds at much higher intensity to cells with 10-fold lower concentration compared to the Fab. FIG. 11B—A histogram depicting blocking of GAD555-567 specific, DR4 restricted T cell response. The G3H8Fab and IgG compete with the autoreactive TCR on the GAD555-567 hybridoma and inhibit the GAD-specific response in a dose-dependent manner. IgG inhibition is >10 fold more efficient compared to the Fab inhibition. FIG. 11C—A histogram depicting blocking of HA-306-318 specific, DR4 restricted T cell response by HB298 but not with G3H8. G3H8 IgG Ab did not inhibit other T cell specificity against a flu peptide (HA-306-318). This is compared to the inhibition obtained by control anti-DR mAb (HB298). These results demonstrate the specificity of the G3H8 antibody towards the DR4/GAD-555-567 and not to unrelated complexes (e.g., of flu).

### DESCRIPTION OF SPECIFIC EMBODIMENTS OF THE INVENTION

The present invention, in some embodiments thereof, relates to isolated complexes of MHC class II and diabetes-associated autoantigenic peptides, isolated high affinity entities such as antibodies which specifically bind to same and, more particularly, but not exclusively, to uses thereof for diagnosing and treating type I diabetes.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have generated MHC class II-diabetes-associated autoantigenic peptides complexes which were used for the isolation of T-cell receptor like antibodies useful for studying antigen presentation during progression of type I diabetes as well as for diagnosing and treating type I diabetes.

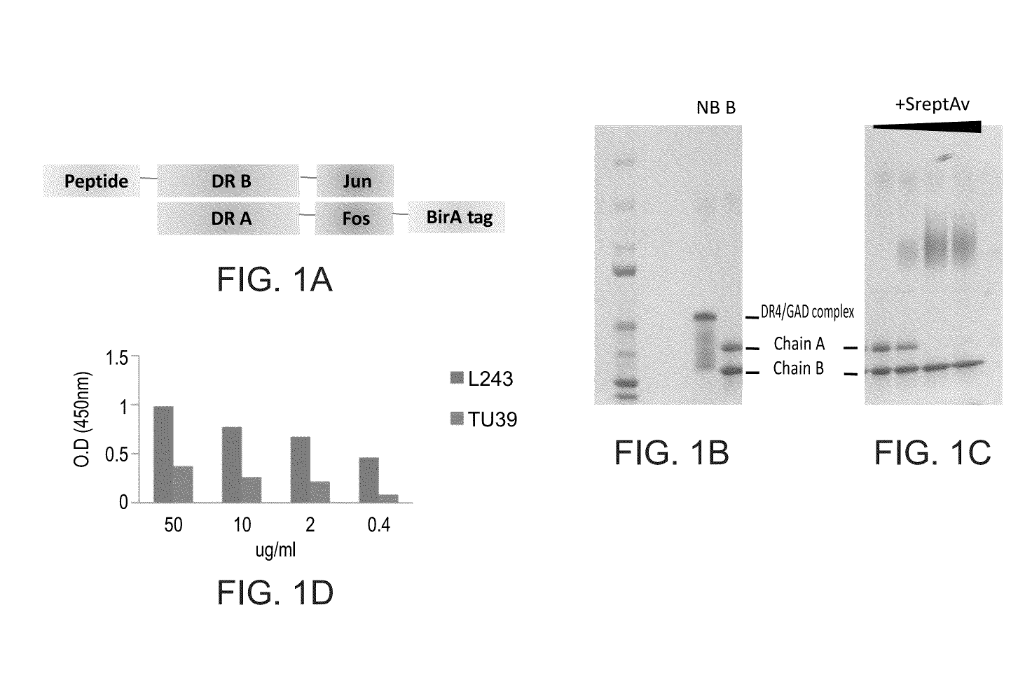

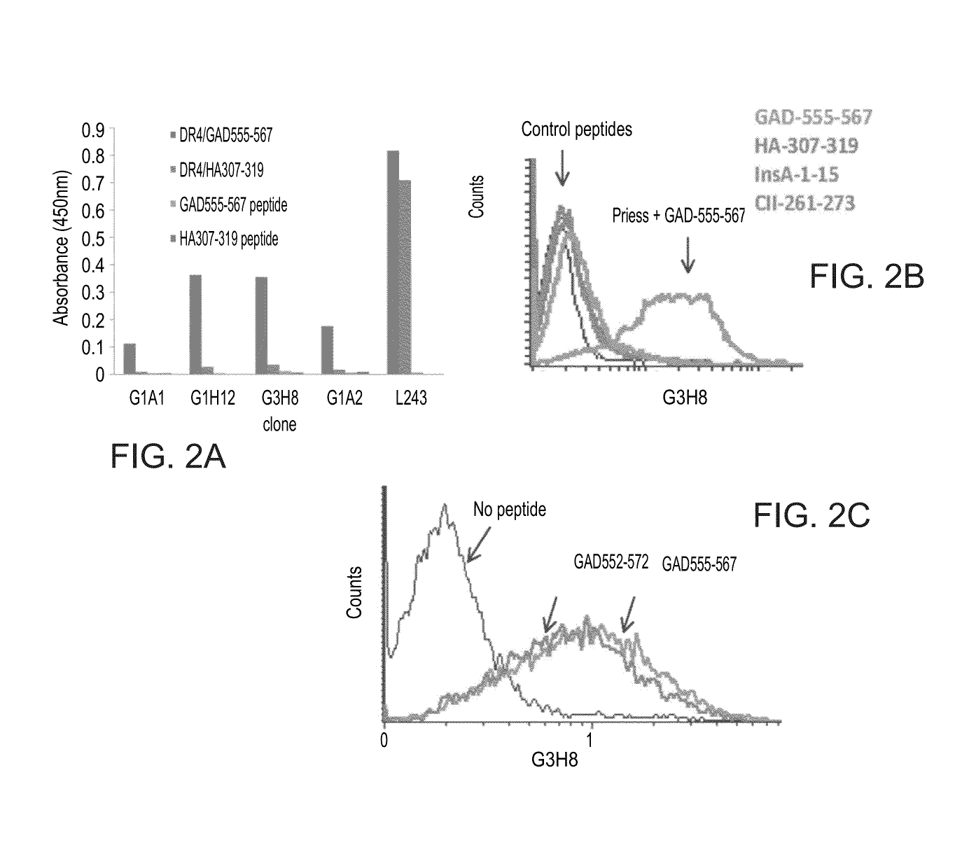

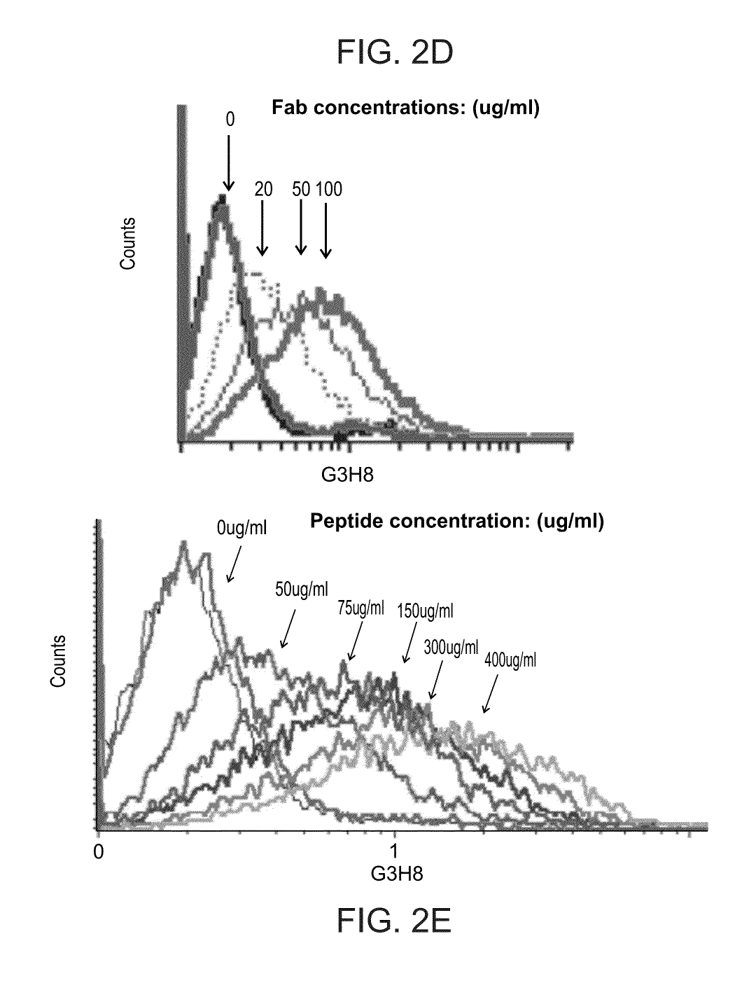

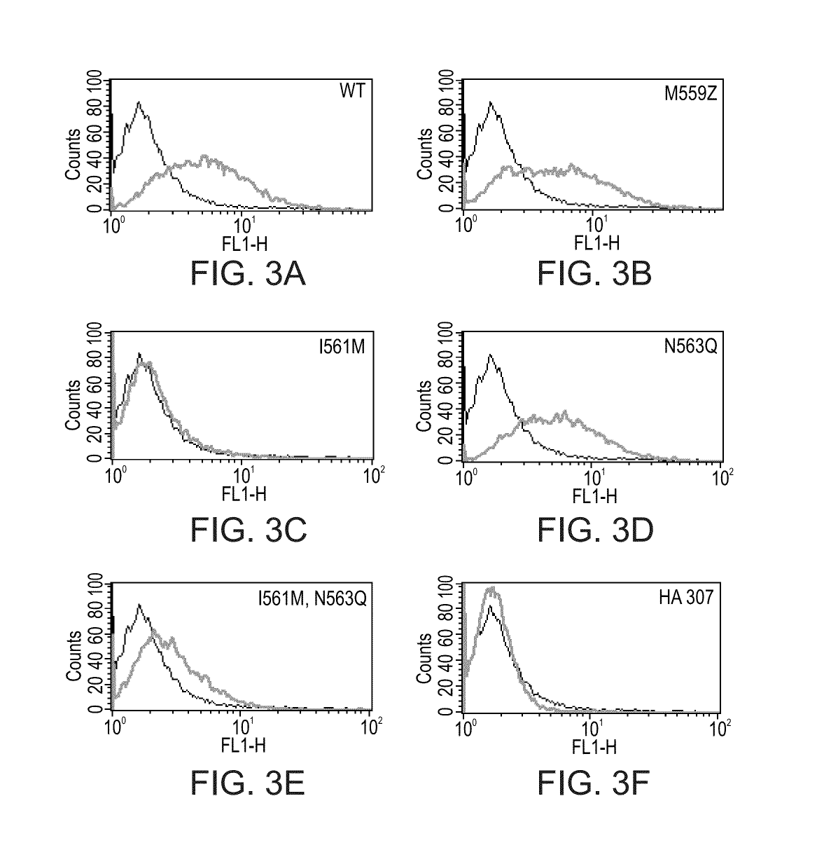

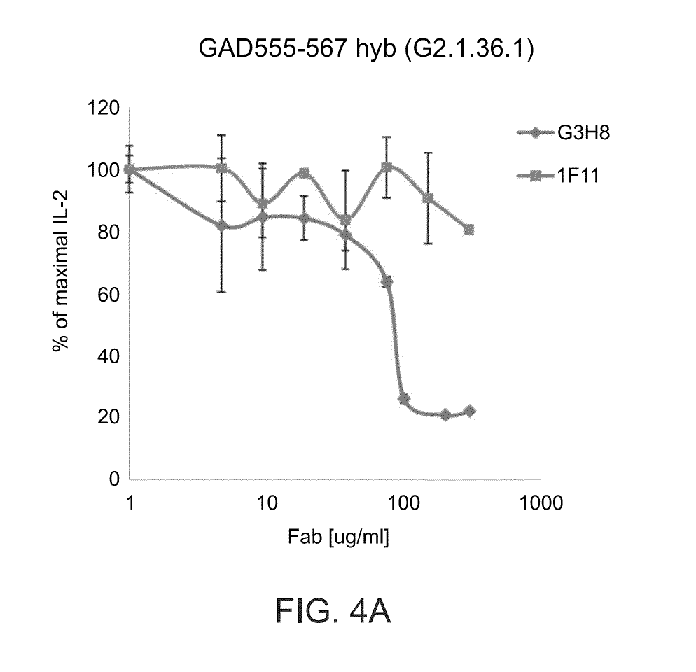

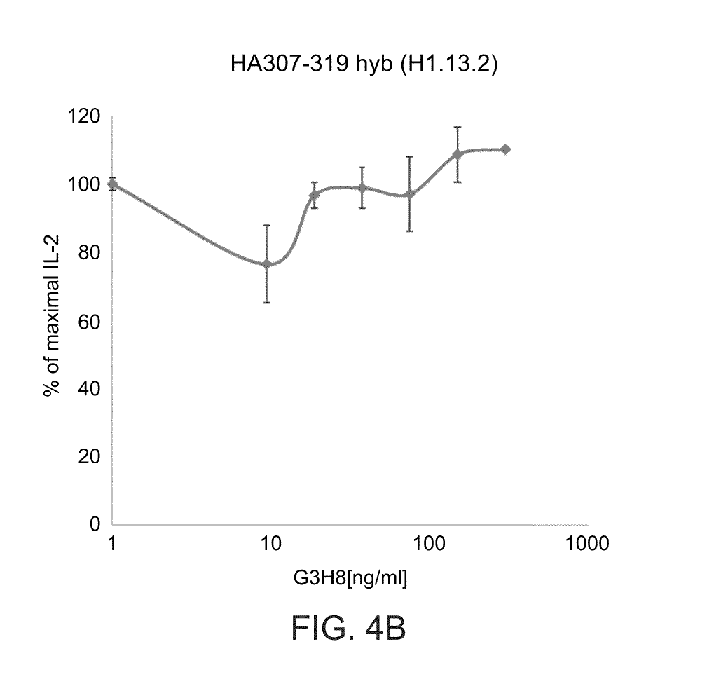

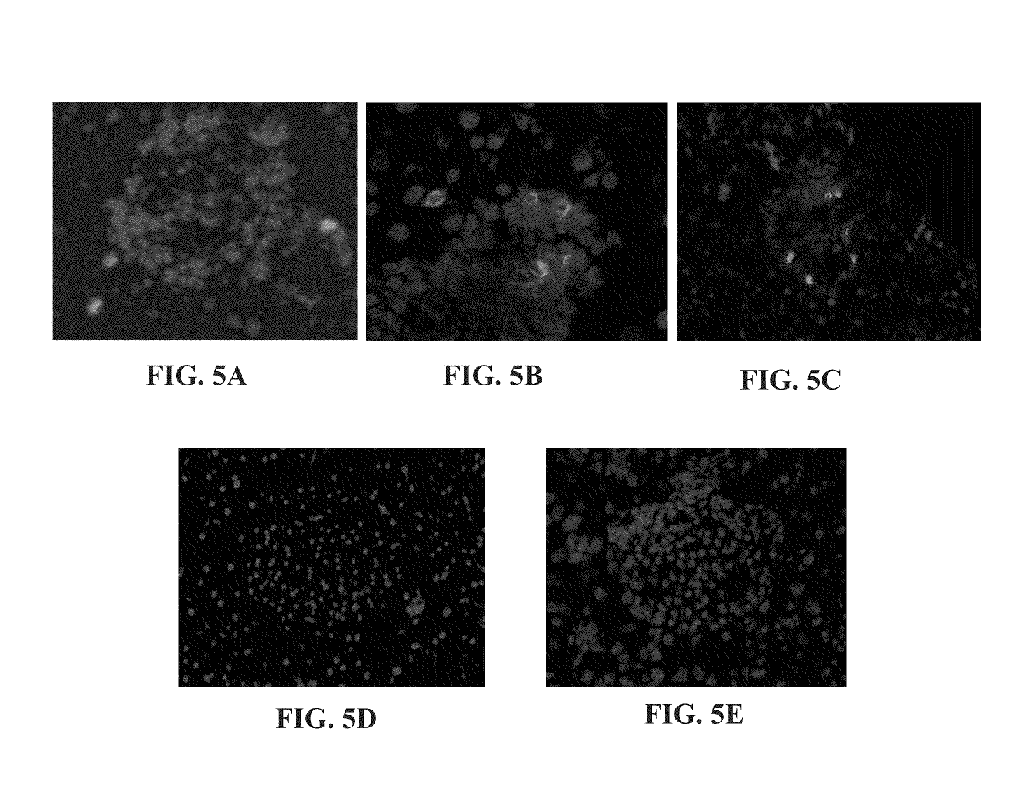

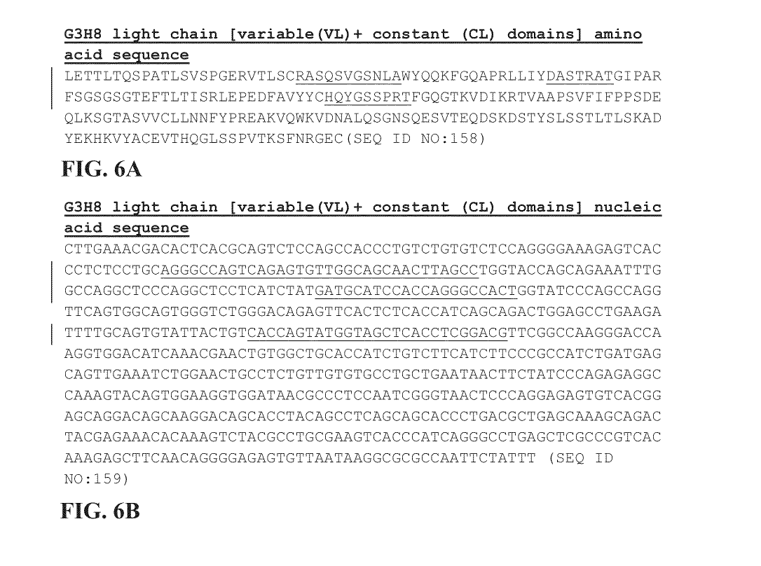

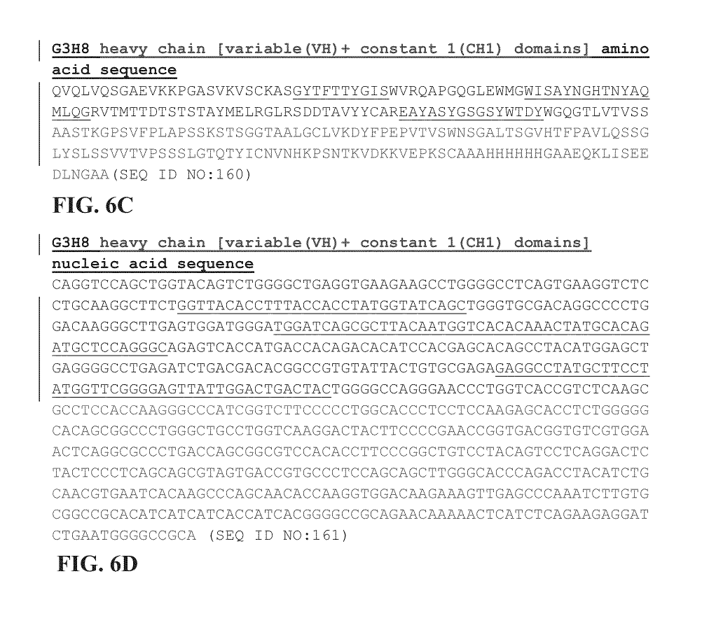

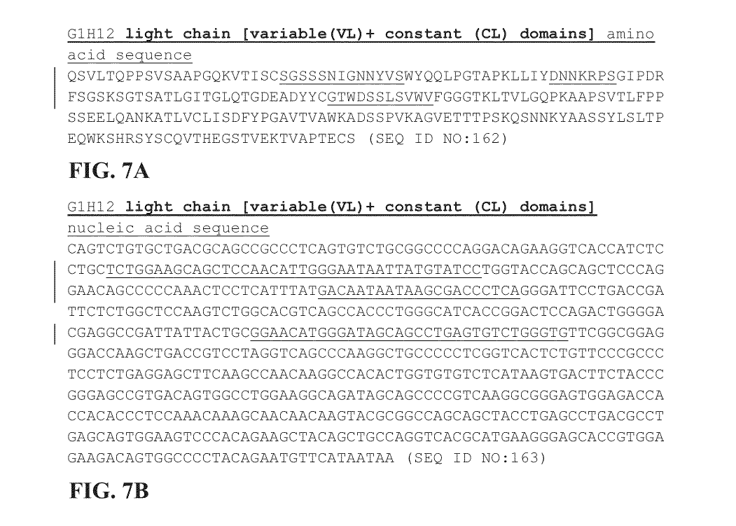

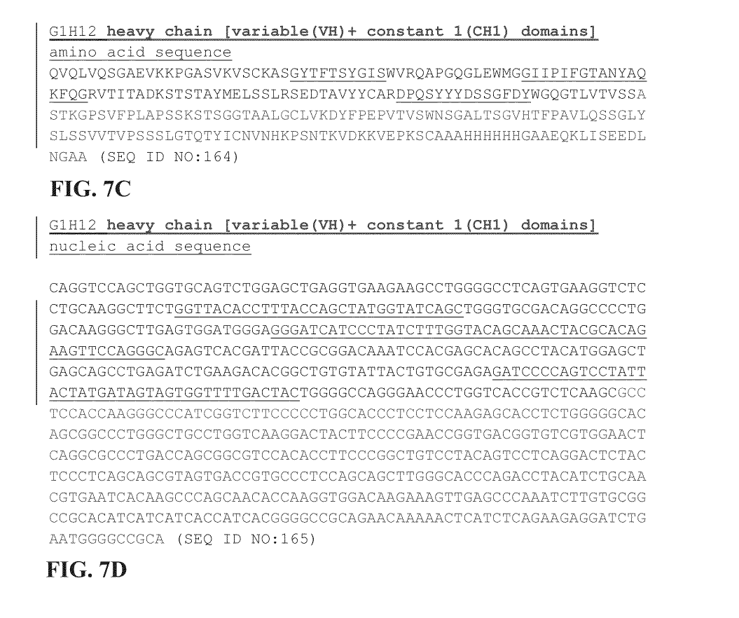

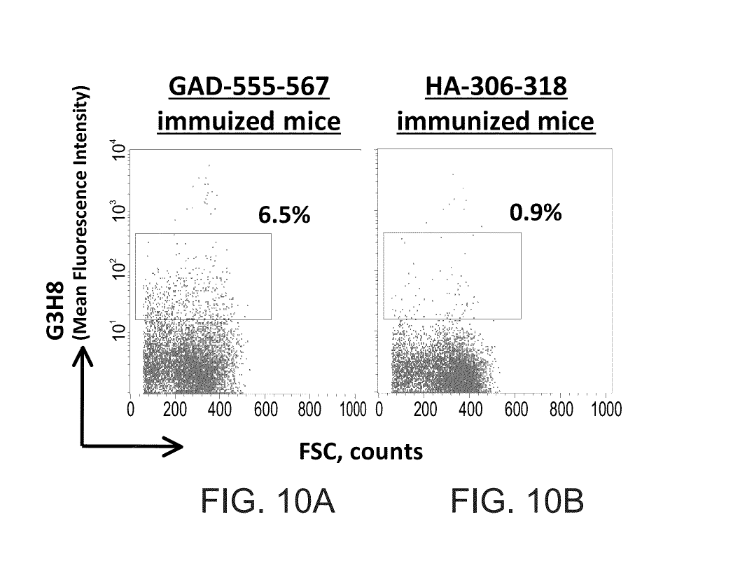

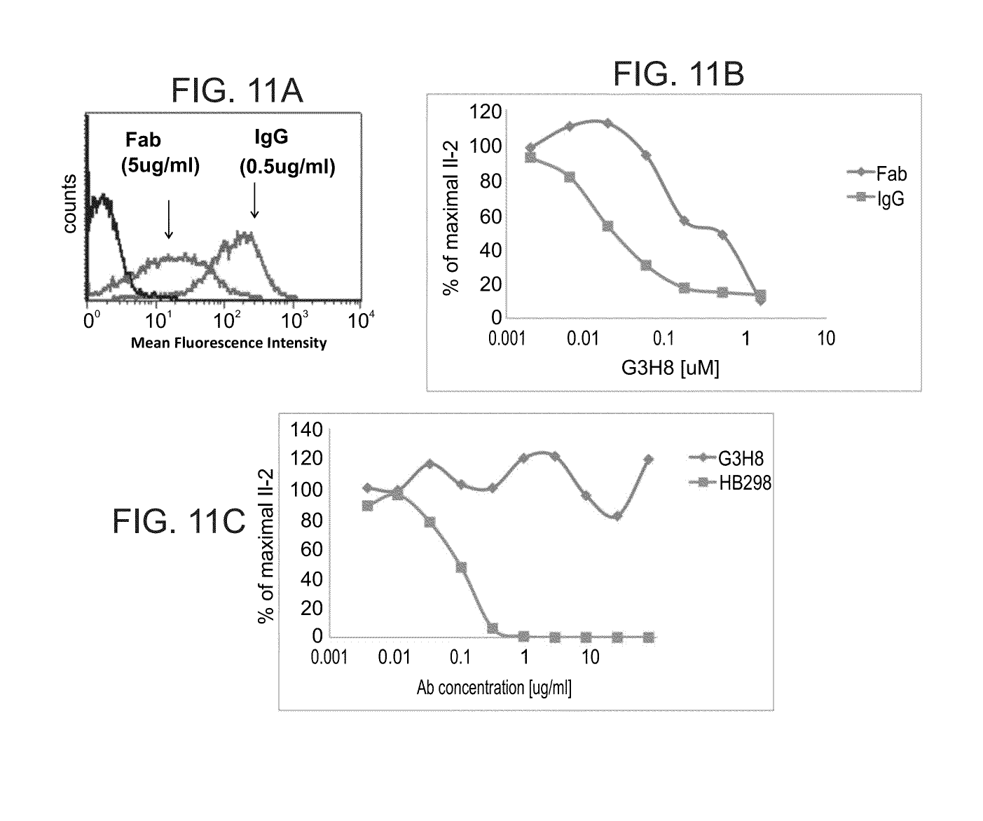

As described in the Examples section which follows, the present inventors generated an isolated complex of MHC class II and a GAD₅₅₅₋₅₆₇ antigenic peptide in which the antigenic peptide is covalently linked to the N-terminus of the MHC class II beta chain (FIG. 1A, Example 1). The MHC class II/GAD peptide complex was used for isolating specific soluble antibodies (e.g., Fabs) which specifically bind the MHC class II (e.g., DR4) when bound to the GAD₅₅₅₋₅₆₇ antigenic peptide both in vitro and in the native conformation (e.g., when presented on cells), but not to the MHC class II in the absence of the specific antigenic peptide (FIGS. 2A-B). In addition, these antibodies were found capable of binding to cells loaded with the naturally T1D-associated epitope GAD₅₅₂₋₅₇₂ (SEQ ID NO:203) (FIG. 2C, Example 1 and data not shown); exhibit T-cell receptor like specificity at various antibody's concentrations (FIG. 2D, Example 1) and various antigenic-peptide concentrations (FIG. 2E, Example 1), with increasing antibody's staining in correlation with increases in the total MHC class II/antigenic peptide complexes on the cells. These results show that the isolated antibodies can be used in quantifying antigen presentation of antigen-presenting cells-of-interest. In addition, as described in Example 2, the isolated antibodies of the invention exhibit fine specificity to their targeted complex and differentially bind to complexes including a wild type peptide, but not to complexes with a mutated amino acid at position P5 of the MHC class II-GAD restricted antigenic peptide (FIGS. 3A-E). Furthermore, as shown in Example 3, G3H8Fab was found to inhibit—80% response of G2.1.36.1 T cell hybridoma specific to GAD-555-567 restricted by HLA-DR*0401 (FIG. 4A) but not the H1.13.2 hybridoma response to HA307-319 peptide restricted by HLA-DR*0401 (FIG. 4B), thus demonstrating an antigen-specific blocking of autoreactive T cells response to the autoreactive GAD-epitope by G3H8 Fab. In addition, as described in Example 4, the G3H8Fab specifically bound to the MHC class II-GAD₅₅₅₋₅₆₇ complexes in islets of B7/DR4 diabetic mice (FIGS. 5A-C) and in infiltrated islets of B7/DR4 pre-diabetic mice (data not shown) but not to islets of C57B6 control mice (FIGS. 5D-E). Moreover, as described in Example 6, a whole IgG G3H8 antibody was generated and was shown to be specific towards cells presenting the HLA-DR4-GAD555-567 complexes ex vivo (FIGS. 10A-B), with enhanced binding as compared to the G3H8Fab (FIG. 11A), with higher potency (FIG. 11B) while maintaining the unique TCR-like specificity (FIG. 11C). Altogether, these results demonstrate the specificity of the antibodies, their use in diagnosing diabetes at early stages and the accessibility of the antibodies to the islets infiltrating APC, which is essential for therapeutic purposes, for blocking specific MHC class II/peptide events associated with the progression of the disease.

Thus, according to an aspect of some embodiments of the invention, there is provided an isolated complex comprising a major histocompatibility complex (MHC) class II and a type I diabetes-associated autoantigenic peptide.

As used herein the term “isolated” refers to at least partially separated from the natural environment e.g., the human body.

According to some embodiments the isolated complex is soluble.

As used herein the phrase “major histocompatibility complex (MHC)” refers to a complex of antigens encoded by a group of linked loci, which are collectively termed H-2 in the mouse and human leukocyte antigen (HLA) in humans. The two principal classes of the MHC antigens, class I and class II, each comprise a set of cell surface glycoproteins which play a role in determining tissue type and transplant compatibility. In transplantation reactions, cytotoxic T-cells (CTLs) respond mainly against foreign class I glycoproteins, while helper T-cells respond mainly against foreign class II glycoproteins.

MHC class II molecules are expressed in professional antigen presenting cells (APCs) such as macrophages, dendritic cells and B cells. Each MHC class II molecule is a heterodimer composed of two homologous subunits, alpha chain (with α1 and α2 extracellular domains, transmembrane domain and short cytoplasmic tail) and beta chain (with β1 and β2 extracellular domains, transmembrane domain and short cytoplasmic tail). Peptides, which are derived from extracellular proteins, enter the cells via endocytosis, are digested in the lysosomes and further bind to MHC class II molecules for presentation on the membrane.

Various MHC class II molecules are found in humans. Examples include, but are not limited to HLA-DM, HLA-DO, HLA-DP, HLA-DQ (e.g., DQ2, DQ4, DQ5, DQ6, DQ7, DQ8, DQ9), HLA-DR (e.g., DR1, DR2, DR3, DR4, DR5, DR7, DRB, DR9, DR10, DR11, DR12, DR13, DR14, DR15, and DR16).

Non-limiting examples of DQ A1 alleles include 0501, 0201, 0302, 0301, 0401, 0101, 0102, 0104, 0102, 0103, 0104, 0103, 0102, 0303, 0505 and 0601.

Non-limiting examples of DQ B1 alleles include 0201, 0202, 0402, 0501, 0502, 0503, 0504, 0601, 0602, 0603, 0604, 0609, 0301, 0304, 0302 and 0303.

Non-limiting examples of DPA1 alleles include 01, e.g., 0103, 0104, 0105, 0106, 0107, 0108, 0109; 02, e.g., 0201, 0202, 0203; 03 e.g., 0301, 0302, 0303, 0401.

Non-limiting examples of DPB1 alleles include 01, e.g., 0101, 0102; 02 e.g., 0201, 0202, 0203; 03; 04, e.g., 0401, 0402, 0403; 05, e.g., 0501, 0502; 06; 08, e.g., 0801, 0802; 09, e.g., 0901, 0902; 10, e.g., 1001, 1002; 11 e.g., 1101, 1102; 13, e.g., 1301, 1302; 14, e.g., 1401, 1402; 15, e.g., 1501, 1502; 16, e.g., 1601, 1602; 17, e.g., 1701, 1702; 18, e.g., 1801, 1802; 19, e.g., 1901, 1902; 20, e.g., 2001, 2002; 21; 22; 23; 24; 25; 26, e.g., 2601, 2602; and 27.

Non-limiting examples of DP haplotypes include HLA-DPA1*0103/DPB1*0401 (DP401); and HLA-DPA1*0103/DPB1*0402 (DP402).

Non-limiting examples of DR B1 alleles include 0101, 0102, 0103, 0301, 0401, 0407, 0402, 0403, 0404, 0405, 0701, 0701, 0801, 0803, 0901, 1001, 1101, 1103, 1104, 1201, 1301, 1302, 1302, 1303, 1401, 1501, 1502, 1601 alleles.

Non-limiting examples of DR-DQ haplotypes include DR1-DQ5, DR3-DQ2, DR4-DQ7, DR4-DQ8, DR7-DQ2, DR7-DQ9, DR8-DQ4, DR8-DQ7, DR9-DQ9, DR10-DQ5, DR11-DQ7, DR12-DQ7, DR13-DQ6, DR13-DQ7, DR14-DQ5, DR15-DQ6, and DR16-DQ5.

According to some embodiments of the invention, the beta chain of the MHC class II complex is DR-B1*0401 (SEQ ID NO:212; native DR-B1*0401 molecule)

According to some embodiments of the invention, the alpha chain of the MHC class II is DR-A1*0101 (SEQ ID NO:211; native DR-A1*0101 molecule).

As used herein the phrase “type I diabetes-associated autoantigenic peptide” refers to an antigen derived from a self protein (i.e., an endogenous protein), which is expressed in pancreatic cells such as beta cells of the pancreas, and against which an inflammatory response is elicited as part of an autoimmune inflammatory response.

It should be noted that a type I diabetes-associated autoantigenic peptide is an MHC class II-restricted peptide, which when presented on antigen presenting cells (APCs) is recognized by specific T cells. Such a presentation by APCs generates an inflammatory response that can activate and recruit T cell and B cell responses against beta cells, including the generation of cytotoxic T cells and antibodies which kill and destroy beta cells and thus lead to a decreased insulin production.

According to some embodiments of the invention, the diabetes-associated autoantigenic peptide is a beta-cell autoantigenic peptide.

According to some embodiments of the invention, the diabetes-associated autoantigenic peptide is derived from a polypeptide selected from the group consisting of preproinsulin (amino acids 1-110 of GenBank Accession No. NP_{—}000198, SEQ ID NO:213), proinsulin (amino acids 25-110 of GenBank Accession No. NP_{—}000198, SEQ ID NO:223), Glutamic acid decarboxylase (GAD, GenBank Accession No. NP_{—}000809.1, SEQ ID NO:214), Insulinoma Associated protein 2 (IA-2, GenBank accession No. NP_{—}115983) SEQ ID NO:215), IA-2β [also referred to as phogrin, GenBank Accession No. NP_{—}570857.2 (SEQ ID NO:221), NP_{—}570858.2 (SEQ ID NO:270), NP_{—}002838.2 (SEQ ID NO:222)], Islet-specific Glucose-6-phosphatase catalytic subunit-Related Protein [IGRP; GeneID: 57818, GenBank Accession No. NP_{—}066999.1, glucose-6-phosphatase 2 isoform 1 (SEQ ID NO:216) and GenBank Accession No. NP_{—}001075155.1, glucose-6-phosphatase 2 isoform 2 (SEQ ID NO:217)], chromogranin A (GenBank Accession No. NP_{—}001266 (SEQ ID NO:218), Zinc Transporter 8 (ZnT8 (GenBank Accession NO. NP_{—}776250.2, SEQ ID NO:219), Heat Shock Protein-60 (GenBank Accession No. NP_{—}955472.1; SEQ ID NO:220), and Heat Shock Protein-70 (GenBank Accession No. NP_{—}005337.2 (SEQ ID NO:271) and NP_{—}005336.3 (SEQ ID NO:224).

According to some embodiments of the invention, the diabetes-associated autoantigenic peptide is a GAD derived autoantigenic peptide selected from the group consisting of SEQ ID NOs:1-45 and 260, 267-268 (Table 3, Example 5 of the Examples section).

According to some embodiments of the invention, the diabetes-associated autoantigenic peptide is a ZnT8 derived autoantigenic peptide selected from the group consisting of SEQ ID NOs: 46-53 (Table 3, Example 5 of the Examples section).

According to some embodiments of the invention, the diabetes-associated autoantigenic peptide is a IA-2 derived autoantigenic peptide selected from the group consisting of SEQ ID NOs: 54-115 (Table 3, Example 5 of the Examples section).

According to some embodiments of the invention, the diabetes-associated autoantigenic peptide is a preproinsulin derived autoantigenic peptide selected from the group consisting of SEQ ID NOs:116-136 (Table 4, Example 5 of the Examples section).

According to some embodiments of the invention, the diabetes-associated autoantigenic peptide is a HSP-60 derived autoantigenic peptide selected from the group consisting of SEQ ID NOs: 137-144 (Table 4, Example 5 of the Examples section).

According to some embodiments of the invention, the diabetes-associated autoantigenic peptide is a HSP-70 derived autoantigenic peptide selected from the group consisting of SEQ ID NOs: 145-153 (Table 3, Example 5 of the Examples section).

According to some embodiments of the invention, the diabetes-associated autoantigenic peptide is a IGRP derived autoantigenic peptide selected from the group consisting of SEQ ID NOs:154-157 (Table 5, Example 5 of the Examples section).

Further description of type I diabetes-associated autoantigenic peptides can be found in Lieberman S M, DiLorenzo TP, 2003. A comprehensive guide to antibody and T-cell responses in type 1 diabetes. Tissue Antigens, 62:359-77; Liu J, Purdy L E, Rabinovitch S, Jevnikar A M, Elliott J F. 1999, Major DQ8-restricted T-cell epitopes for human GAD65 mapped using human CD4, DQA1*0301, DQB1*0302 transgenic IA(null) NOD mice, Diabetes, 48: 469-77; Di Lorenzo T P, Peakman M, Roep B O. 2007, Translational mini-review series on type 1 diabetes: Systematic analysis of T cell epitopes in autoimmune diabetes. Clin Exp Immunol. 148:1-16; Stadinski et al Immunity 32:446, 2010; each of which is fully incorporated herein by reference).

Since the amino acid sequence of the autoantigen may vary in length between the same or different MHC class II alleles, the length of the autoantigenic peptides according to some embodiments of the invention may vary from at least 6 amino acids, to autoantigenic peptides having at least 8, 10, 25, or up to 30 amino acids.

According to some embodiments of the invention, the diabetes-associated autoantigenic peptide includes a core amino acids of at least 6 amino acids, e.g., at least 7, at least 8, at least 9 and more.

According to some embodiments of the invention, the length of the diabetes-associated autoantigenic peptide does not exceed about 100 amino acids, e.g., does not exceed about 50 amino acids, e.g., does not exceed about 30 amino acids.

According to some embodiments of the invention, the diabetes-associated autoantigenic peptide comprises the amino acid sequence selected from the group consisting of SEQ ID NOs:1-157 260, and 267-268 and no more than 30 amino acids in length.

According to some embodiments of the invention, the diabetes-associated autoantigenic peptide is selected from the group consisting of SEQ ID NOs:1-157, 260, and 267-268.

According to some embodiments of the invention, the length of the diabetes-associated autoantigenic peptide includes at least 6 and no more than 30 amino acids.

In addition, it should be noted that although some amino acids in each autoantigenic peptide are conserved between various alleles of MHC class II and cannot be substituted, other amino acids can be substituted with amino acids having essentially equivalent specificity and/or affinity of binding to MHC molecules and resulting in equivalent T cell epitope as the amino acid sequences shown in the exemplary autoantigens described above and in Tables 3-5 (Example 5 of the Examples section). Thus, in each autoantigenic peptide there are at least six amino acids constituting a core amino acid which are required for recognition with the respective MHC class II molecule. Identification of the core amino acids for each autoantigenic peptide can be done experimentally, e.g., by mutagenesis of the amino acids constituting the autoantigenic peptide and detection of: (i) binding to the restricted MHC class II molecules; (ii) Stimulating the restricted T cell response. For example, for the GAD₅₅₅₋₅₆₇ the core amino acids are the amino acids at positions 556-565. The core amino acid sequence consists of anchor residues and the T-cell receptor (TCR) contact residues. Anchor residues in the sequence NFFRMVISNPAAT (SEQ ID NO:12) are the P1 (F557), P4 (V560), P6 (S562), and P9 (A565) MHC pocket-binding residues. TCR contact residues in the sequence NFFRMVISNPAAT (SEQ ID NO:12) are at positions F556, R558, M559, I561, N563. Accordingly, the core amino acids of the GAD555-567 autoantigenic peptide are GAD556-565 (FFRMVISNPA, SEQ ID NO:260).

The invention according to some embodiments thereof also concerns peptide variants whose sequences do not completely correspond with the aforementioned amino acid sequences but which only have identical or closely related “anchor positions”. The term “anchor position” in this connection denotes an essential amino acid residue for binding to a MHC class II complex (e.g., DR1, DR2, DR3, DR4 or DQ). The anchor position for the DRB1*0401 binding motif are for example stated in Hammer et al., Cell 74 (1993), 197-203. Such anchor positions are conserved in the diabetes-associated autoantigenic peptide or are optionally replaced by amino acid residues with chemically very closely related side chains (e.g. alanine by valine, leucine by isoleucine and visa versa). The anchor position in the peptides according to some embodiments of the invention can be determined in a simple manner by testing variants of the aforementioned specific peptides for their binding ability to MHC molecules. Peptides according to some embodiments of the invention are characterized in that they have an essentially equivalent specificity or/and affinity of binding to MHC molecules as the aforementioned peptides. Homologous peptides having at least 50%, e.g., at least 60%, 70%, 80%, 90%, 95% or more identity to the diabetes-associated autoantigenic peptides described herein are also contemplated by some embodiments of the invention.

It should be noted that each of the above described diabetes-associated autoantigenic peptides can be complexed with an MHC class II allele. Such MHC class II specific alleles are known in the art. Non-limiting examples of MHC class II alleles and their restricted autoantigenic peptides are illustrated in Table 3 in Example 5 of the Examples section which follows.

As used herein the phrase “glutamic acid decarboxylase (GAD)” refers to a family of proteins which are responsible for catalyzing the production of gamma-aminobutyric acid from L-glutamic acid. There are two major GAD enzymes in humans, GAD 65 kDa which is expressed in both brain and pancreas (GeneID 2572; encoded by GenBank accession No. NM_{—}000818.2 (SEQ ID NO:198); NM_{—}001134366.1 (SEQ ID NO:199); NP_{—}000809.1 (SEQ ID NO:200)] and GAD 67 kDa which is expressed in brain [GeneID 2571; encoded by GenBank accession No. NM_{—}000817.2 (SEQ ID NO:201); NP_{—}000808.2 (SEQ ID NO:202)]. GAD 65 kDa has been identified as an autoantibody and an autoreactive T cell target in insulin-dependent diabetes.

According to some embodiments of the invention, the diabetes-associated autoantigenic peptide is GAD₅₅₅₋₅₆₇ (NFFRMVISNPAAT; SEQ ID NO:12).

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2—NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time. According to some embodiments of the invention, but not in all cases necessary, these modifications should exclude anchor amino acids.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the peptides of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

The peptides of the invention are preferably utilized in a linear form, although it will be appreciated that in cases where cyclicization does not severely interfere with peptide characteristics, cyclic forms of the peptide can also be utilized.

The peptides of the invention may include one or more non-natural or natural polar amino acids, including but not limited to serine and threonine which are capable of increasing peptide solubility due to their hydroxyl-containing side chain.

The peptides of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W.H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965. Large scale peptide synthesis is described by Andersson Biopolymers 2000; 55(3):227-50.

According to some embodiments of the invention, the isolated complex which comprises the MHC class II and the type I diabetes-associated autoantigenic peptide has a structural conformation which enables isolation of a high affinity entity which comprises an antigen binding domain capable of specifically binding to a native conformation of a complex composed of the MHC class II and the type I diabetes-associated autoantigenic peptide.

According to some embodiments of the invention, the high affinity entity does not bind to the MHC class II in an absence of the diabetes-associated autoantigenic peptide, wherein the isolated high affinity entity does not bind to the diabetes-associated autoantigenic peptide in an absence of the MHC class II.

The phrase “MHC class II in the absence of the diabetes-associated autoantigenic peptide” as used herein encompasses an empty MHC class II complex (i.e., devoid of any antigenic peptide) as well as an MHC class II complex which is bound to another antigen peptide which is not the diabetes-associated autoantigenic peptide of some embodiments of the invention, e.g., a different MHC class II-restricted antigenic peptide.

The phrase “diabetes-associated autoantigenic peptide in an absence of the MHC class II” as used herein encompasses the diabetes-associated autoantigenic peptide of some embodiments of the invention when not bound to the MHC class II complex as well as to the diabetes-associated autoantigenic peptide of some embodiments of the invention when bound to another MHC class II complex, e.g., a different allele of an MHC class II beta or alpha chain than the chain(s) used for forming the complex of some embodiments of the invention.

According to some embodiments of the invention, the isolated complex which comprises the MHC class II and the diabetes-associated autoantigenic peptide does not include an heterologous immunoglobulin (e.g., an Fc, Fab and/or a single chain Fv antibody) attached thereto (either a covalent or a non-covalent attachment to the MHC class II molecules, e.g., via the C′-terminus of the MHC class II molecules).

In order to isolate high affinity entities which can specifically bind to MHC class

II/diabetes-associated autoantigenic peptides having a native structural conformation, the isolated MHC/peptide complexes should be generated such that a correct folding of the MHC class II alpha and beta chains with the antigenic peptide occurs. It should be noted that for preparation of a recombinant complex of MHC class II and a restricted antigen peptide the extracellular domains of the alpha and beta chains are required.

When expressed in eukaryotic cells, the signal peptide of the MHC class II molecules is cleaved post translationally, thus obtaining a mature protein. To enable correct folding of the antigenic peptide within the MHC class II molecules, the antigenic peptide should be covalently attached close to the N-terminus of the extracellular domain of the mature MHC class II beta chain.

According to some embodiments of the invention, the structural conformation is obtainable when the diabetes-associated autoantigenic peptide is covalently conjugated or bound to the extracellular domain of the mature beta chain of the MHC class II.

According to some embodiments of the invention, the structural conformation is obtained when the diabetes-associated autoantigenic peptide is covalently conjugated or bound to the extracellular domain of the mature beta chain of the MHC class II.

According to some embodiments of the invention, the diabetes-associated autoantigenic peptide is covalently bound at a C terminus thereof to an N-terminus of an extracellular domain of the MHC class II.

As used herein the phrase “covalently bound” (or conjugated) refers to being part of the polypeptide chain of the mature beta chain. Such a covalent conjugation can be achieved by translationally fusing the coding sequence of the diabetes-associated autoantigenic peptide to the coding sequence of the extracellular domain of the beta chain MHC class II molecule.

According to some embodiments of the invention, the diabetes-associated autoantigenic peptide is covalently embedded between amino acids 1-6 of an extracellular domain of the beta chain of the MHC class II.

As used herein the phrase “covalently embedded between” refers to being covalently bound within an amino acid sequence (a polypeptide).

According to some embodiments of the invention, the diabetes-associated autoantigenic peptide is covalently embedded between amino acids 1-2, 2-3, 3-4, 4-5, or 5-6 of the extracellular domain of the beta chain of the MHC class II.

Thus, the diabetes-associated autoantigenic peptide can be embedded after the first, second, third, forth or fifth amino acid position of the mature extracellular domain of the beta chain of the MHC class II.

According to some embodiments of the invention, the diabetes-associated autoantigenic peptide is covalently attached after the third amino acid of the mature MHC class II beta chain (i.e., between the third and forth amino acids of the mature MHC class II beta chain).

According to some embodiments of the invention, the diabetes-associated autoantigenic peptide is flanked at a C-terminus thereof by a linker peptide. The linker peptide can be selected according to the expression system used for preparing the recombinant MHC class II-antigenic peptide.

Usually, the linker peptide confers flexibility to the mature beta chain and enables the folding of the conjugated antigenic peptide within the peptide-binding grooves within the MHC class II molecules.

In some embodiments of the invention, the linker peptide comprises a site for an enzymatic cleavage of the recombinant protein. Cleavage can be done in vivo (i.e., within a living organism), ex vivo (when cell of an organism are cultured) or in vitro.

According to some embodiments of the invention, the linker peptide may include a thrombin cleavage site. For example, a linker peptide may comprise a thrombin cleavage site (e.g., the sequence LVPRGS) flanked by two sequences which increase flexibility of the recombinant protein such as GGGGS.

Following are non-limiting examples of linker peptides which can be covalently conjugated to the diabetes-associated autoantigenic peptide complexes:

(1) A linker peptide comprising the Glycine (G)—Serine (S) pair of amino acids being repeated between one to 30 times [GS]n (wherein n=1-30) (SEQ ID NO:272).

(2). A linker peptide comprising the GGGGS sequence being repeated between one to 6 times [GGGGS]n (wherein n=1-6) (SEQ ID NO:261).

(3) A linker peptide GGGSLVPRGSGGGGS (SEQ ID NO:262);

(4) A linker peptide GGGGSLVPRGSGGGGS (SEQ ID NO:263).

The linker peptide can be translationally fused to the diabetes-associated autoantigenic peptide and to the extracellular domain of the mature beta chain MHC class II. For example, the C-terminus of the diabetes-associated autoantigenic peptide is fused directly to the N-terminus of the linker peptide; and the C-terminus of the linker peptide is fused directly to the N-terminus or to an amino acid position between 1-6 of the N-terminal end of the mature beta chain extracellular domain.

In addition, in order to form a non-covalent complex between the alpha and beta chains of the MHC class II, each of the extracellular domains of the alpha and beta chains comprises a member of a binding pair, which upon interaction with the other member forms a binding pair.

Non-limiting examples of such binding pairs include the leucine-zipper dimerization domains of Jun-Fos binding pairs and the acidic (AZ) and basic (BZ) leucin zipper motives which form a stable protein complex.

According to some embodiments of the invention, the beta chain of the MHC class II comprises a first member of a binding pair which upon expression in eukaryotic cells binds to a second member of the binding pair, wherein the second member is comprised in an alpha chain of the MHC class II, wherein the beta chain and the alpha chain form the MHC class II.

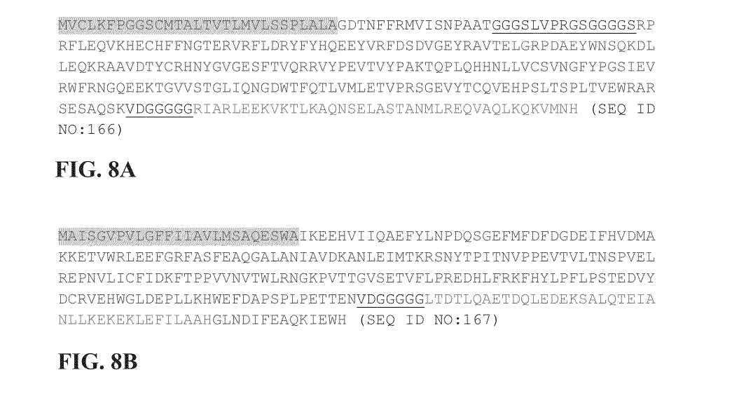

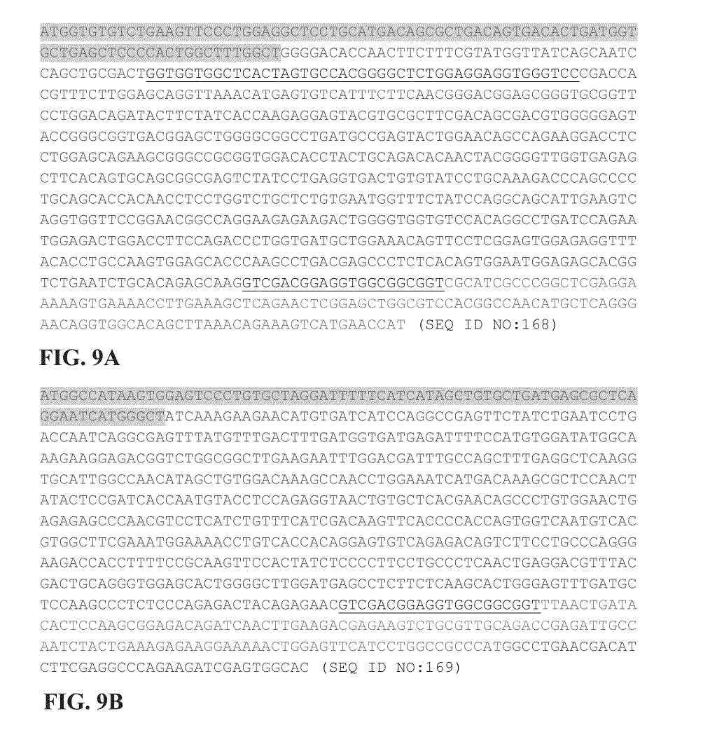

For example, as described in the Examples section which follows, the MHC class II complex of some embodiments of the invention was generated by expressing in a host cell (e.g., S2 cells) a polynucleotide which comprises a nucleic acid sequence encoding a diabetes-associated autoantigenic peptide (e.g., GAD peptide) which is translationally fused to a nucleic acid sequence encoding an MHC class II beta chain (e.g., DR-B1*0401; SEQ ID NO:212) such that the encoded antigenic peptide is fused between the third and forth amino acid positions of the beta chain (of the mature extracellular domain of the beta chain). As further shown in FIGS. 8A-B, the antigenic peptide is covalently fused to a linker peptide which is bound directly to the forth amino acid position (4^{th} amino acid) of the mature extracellular domain of the beta chain.

The phrases “translationally fused” and “in frame” are interchangeably used herein to refer to polynucleotides which are covalently linked to form a single continuous open reading frame spanning the length of the coding sequences of the linked polynucleotides. Such polynucleotides can be covalently linked directly or preferably indirectly through a spacer or linker region.

According to an aspect of some embodiments of the invention, there is provided an isolated polynucleotide comprising a first nucleic acid sequence encoding an extracellular domain of an MHC class II beta chain [e.g., DR-B1*0401; (SEQ ID NO:264 for the amino acid sequence) and; (SEQ ID NO:265 for the nucleic acid sequence)] and a second nucleic acid construct encoding a diabetes-associated autoantigenic peptide [e.g., GAD-peptide NFFRMVISNPAAT (SEQ ID NO:12), AACTTCTTTCGTATGGTTATCAGCAATCCAGCTGCGACT (SEQ ID NO:266) for the nucleic acid sequence encoding the GAD-peptide], wherein the second nucleic acid construct being translationally fused upstream of the first nucleic acid construct or between the nucleic acid sequence encoding amino acids 1-6 of the extracellular domain.

According to some embodiments of the invention, the isolated polynucleotide further comprises a nucleic acid sequence encoding a linker peptide being translationally fused downstream of the second nucleic acid sequence.

According to some embodiments of the invention, the first nucleic acid sequence and the second nucleic acid sequence are connected via a nucleic acid sequence encoding a linker peptide (GGGSLVPRGSGGGGS; SEQ ID NO:262).

According to some embodiments of the invention, the isolated polynucleotide further comprises a third nucleic acid sequence encoding a first member of a binding pair [(e.g., Jun, the amino acid sequence set forth in SEQ ID NO:195 (RIARLEEKVKTLKAQNSELASTANMLREQVAQLKQKVMNH)] which upon expression in eukaryotic cells binds to a second member of the binding pair.

According to some embodiments of the invention, the third nucleic acid sequence encoding a first member of a binding pair is translationally fused downstream of the first nucleic acid sequence encoding an MHC class II beta chain.

According to some embodiments of the invention, the first member of binding pair (e.g., Jun amino acid sequence) is connected via a short peptide linker to the MHC class II beta chain. A non-limiting example of such a linker is set forth in SEQ ID NO:170 (VDGGGGG).

According to an aspect of some embodiments of the invention, there is provided a nucleic acid system comprising:

(i) a first polynucleotide comprising a first nucleic acid sequence encoding an MHC class II beta chain and a second nucleic acid construct encoding a diabetes-associated autoantigenic peptide, wherein the second nucleic acid construct being translationally fused upstream of the first nucleic acid construct; and a third nucleic acid sequence encoding a first member of a binding pair which upon expression in eukaryotic cells binds to a second member of the binding pair; and

(ii) a second polynucleotide which comprises a forth nucleic acid sequence encoding an MHC class II alpha chain [e.g., DR-A1*0101; amino acids 1-217 of SEQ ID NO:167 (of the recombinant molecule); and nucleic acids 1-651 of SEQ ID NO:169].

According to some embodiments of the invention, the second polynucleotide further comprises a fifth nucleic acid sequence encoding the second member of the binding pair [e.g., Fos, the amino acid sequence set forth in SEQ ID NO:196 (LTDTLQAETDQLEDEKSALQTEIANLLKEKEKLEFILAAH)].

According to some embodiments of the invention, the fifth nucleic acid sequence encoding the second member of the binding pair is translationally fused downstream of the forth nucleic acid sequence encoding the MHC class II alpha chain.

According to some embodiments of the invention, the Fos amino acid sequence is connected via a short peptide linker to the MHC class II alpha chain. A non-limiting example of such a linker is set forth in SEQ ID NO:170 (VDGGGGG).

According to some embodiments of the invention, the fifth nucleic acid sequence encoding the second member of the binding pair and the forth nucleic acid sequence encoding an MHC class II alpha chain are connected via a nucleic acid sequence encoding a linker peptide (e.g., VDGGGGG; SEQ ID NO:170).

Non-limiting examples of recombinant beta chain and alpha chain molecules are illustrated in FIGS. 8A-B and 9A-B, and exemplary sequences thereof are provided in SEQ ID NOs: 166-167 and 168-169, respectively.

According to some embodiments of the invention, at least one molecule of the MHC class II complex (i.e., an alpha or beta chain) further comprises an in-frame tag, i.e., a nucleic acid sequence which encodes a peptide capable of being enzymatically modified to include a binding entity. For example, such a peptide can be used for site specific biotinylation using e.g., a biotin protein ligase—Bir A enzyme (AVIDITY). Non-limiting examples of such tags includes the Bir A recognition sequence is set forth by SEQ ID NO:197 (Leu Gly Gly Ile Phe Glu Ala Met Lys Met Glu Leu Arg Asp).

According to some embodiments of the invention, the Bir A recognition sequence for biotinylation is covalently conjugated at the carboxy terminal (C^{t}) of the recombinant alpha chain.

It should be noted that an in-frame tag can be used for isolation of antibodies which specifically bind to the specific MHC-peptide complex, such as using streptavidin.

According to some embodiments of the invention, the MHC class II-peptide complexes forms multimers which are bound by a common binding entity.

For example, multimers (e.g., tetramers) of MHC class II-peptide complexes can be formed using a streptavidin which binds to the biotinylated complexes.

According to an aspect of some embodiments of the invention, there is provided an isolated high affinity entity comprising an antigen binding domain capable of specifically binding a complex composed of a major histocompatibility complex (MHC) class II and a type I diabetes-associated autoantigenic peptide, wherein the isolated high affinity entity does not bind to the MHC class II in an absence of the diabetes-associated autoantigenic peptide, wherein the isolated high affinity entity does not bind to the diabetes-associated autoantigenic peptide in an absence of the MHC class II.

According to some embodiments of the invention, the antigen binding domain is capable of specifically binding to a native conformation of the complex composed of the MHC class II and the type I diabetes-associated autoantigenic peptide.

As used herein the phrase “native conformation” refers to the conformation of the complex when naturally presented on cells, e.g., cells of a mammal, e.g., human cells.

According to some embodiments of the invention, the native conformation comprises the structural conformation of the complex of the type I diabetes-associated autoantigenic peptide and the MHC class II when presented on an antigen presenting cell (APC).

Non-limiting examples of antigen presenting cells which display or present the complex of the MHC class II and the diabetes-associated autoantigenic peptide include macrophages, dendritic cells (DCs) and B-cells.

According to an aspect of some embodiments of the invention, there is provided an isolated high affinity entity comprising an antigen binding domain, the high affinity entity being isolatable by the isolated complex of some embodiments of the invention.

According to an aspect of some embodiments of the invention, there is provided an isolated high affinity entity comprising an antigen binding domain capable of specifically binding to the isolated complex of some embodiments of the invention.

According to some embodiments of the invention, the antigen binding domain of the isolated high affinity entity is capable of specifically binding to a native conformation of a complex composed of the MHC class II and the type I diabetes-associated autoantigenic peptide.

According to some embodiments of the invention, the antigen binding domain of the isolated high affinity entity is further capable of specifically binding to the isolated complex of some embodiments of the invention.

According to an aspect of some embodiments of the invention, there is provided an isolated high affinity entity comprising an antigen binding domain, the antigen binding domain being capable of specifically binding:

(i) a complex composed of a major histocompatibility complex (MHC) class II and a type I diabetes-associated autoantigenic peptide, wherein the isolated high affinity entity does not bind to the MHC class II in an absence of the diabetes-associated autoantigenic peptide, wherein the isolated high affinity entity does not bind to the diabetes-associated autoantigenic peptide in an absence of the MHC class II; and

(ii) a native conformation of a complex composed of an MHC class II and a type I diabetes-associated autoantigenic peptide.

According to an aspect of some embodiments of the invention, there is provided an isolated high affinity entity comprising an antigen binding domain capable of specifically binding to an isolated complex comprising an MHC class II and a type I diabetes-associated autoantigenic peptide, wherein the diabetes-associated autoantigenic peptide being covalently conjugated to the amino terminal (N^{t}) end of a recombinant beta chain of the MHC class II.

According to an aspect of some embodiments of the invention, there is provided an isolated high affinity entity being isolatable by an isolated complex which comprises an MHC class II and a type I diabetes-associated autoantigenic peptide, wherein the diabetes-associated autoantigenic peptide being covalently conjugated at the amino terminal (N^{t}) end of a recombinant beta chain of the MHC class II, wherein an antigen binding domain of the isolated high affinity entity is capable of specifically binding to a native conformation of a complex composed of the MHC class II and the type I diabetes-associated autoantigenic peptide.

According to an aspect of some embodiments of the invention, there is provided an isolated high affinity entity being isolatable by an isolated complex which comprises an MHC class II and a type I diabetes-associated autoantigenic peptide, wherein the diabetes-associated autoantigenic peptide being covalently conjugated at the amino terminal (N^{t}) end of a recombinant beta chain of the MHC class II, wherein an antigen binding domain of the isolated high affinity entity is capable of specifically binding to:

(i) an isolated complex which comprises an MHC class II and a type I diabetes-associated autoantigenic peptide, wherein the diabetes-associated autoantigenic peptide being covalently conjugated at the amino terminal (N^{t}) end of a recombinant beta chain of the MHC class II; and

(ii) a native conformation of a complex composed of the MHC class II and the type I diabetes-associated autoantigenic peptide.

According to an aspect of some embodiments of the invention, there is provided an isolated high affinity entity comprising a complementarity determining regions (CDRs) set forth by SEQ ID NOs:171-173 CDRs 1-3 for light chain; SEQ ID NOs:177-179 CDRs 1-3 for heavy chain (CDRs 1-3 of heavy chain and light chain of G3H8).

According to an aspect of some embodiments of the invention, there is provided an isolated high affinity entity comprising a complementarity determining regions (CDRs) set forth by SEQ ID NOs:183-185 CDRs 1-3 for light chain and SEQ ID NOs:189-191 CDRs 1-3 for heavy chain.

The phrase “high affinity entity” refers to any naturally occurring or artificially produced molecule, composition, or organism which binds to a specific antigen with a higher affinity than to a non-specific antigen.

It should be noted that the affinity can be quantified using known methods such as, Surface Plasmon Resonance (SPR) (described in Scarano S, Mascini M, Turner A P, Minunni M. Surface plasmon resonance imaging for affinity-based biosensors. Biosens Bioelectron. 2010, 25: 957-66), and can be calculated using, e.g., a dissociation constant, Kd, such that a lower Kd reflects a higher affinity.

As described, the high affinity entity binds to a complex comprising an MHC class II and an MHC class II-restricted autoantigen (a diabetes-associated autoantigenic peptide).

According to some embodiments of the invention, the high affinity entity binds to a certain specific complex with a higher affinity as compared to the affinity of the same entity to a similar complex in which at least one of the complex components, i.e., the MHC class II alpha chain, the MHC class II beta chain, and/or the MHC class II-restricted autoantigen being replaced with a component having at least one mutation (substitution, deletion or insertion) with respect to the component of the specific complex.

According to some embodiments of the invention, the mutation is in an amino acid position which is conserved between restricted antigens of various MHC class II alleles.

According to some embodiments of the invention, the high affinity entity exhibits an affinity to a specific antigen which is higher in at least about one order of magnitude as compared to the affinity of the same entity to a non-specific antigen, e.g., at least about 2, at least about 3, at least about 4, at least about 5, at least about 6, at least about 7, at least about 8, at least about 9, at least about 10 orders of magnitudes higher.

According to some embodiments of the invention, the dissociation constant of the high affinity entity to the specific antigen is about 10⁻⁴ M or less, e.g., about 10⁻⁵ M or less, e.g., about 10⁻⁶ M or less, e.g., about 10⁻⁷ or less, e.g., about 10⁻⁸ or less, e.g., about 10⁻⁹ M or less, e.g., about 10⁻¹⁰ M or less.

Non-limiting examples of high affinity entities include an antibody, an antibody fragment, a phage displaying an antibody, a peptibody, a cell-based display entity (e.g., a bacterium or yeast displaying an antibody), and cell-free displaying entity (e.g., a ribosome displaying a peptide or antibody).

Bacteriophages which display antibodies and which can be used according to some embodiments of the invention include M13 and fd filamentous phage, T4, T7, and λ phages.

The techniques of using bacteria (e.g., E. Coli) and yeast for displaying antibodies are well (See e.g., Daugherty P S., et al., 1998. Antibody affinity maturation using bacterial surface display. Protein Engineering 11:825-832; Johan Rockberg et al., Epitope mapping of antibodies using bacterial surface display. Nature Methods 5, 1039-1045 (2008); Sachdev S Sidhu, Full-length antibodies on display, Nature Biotechnology 25, 537-538 (2007); each of which is fully incorporated herein by reference).

Cell-free displaying entities include a ribosome displaying a protein (described in Mingyue He and Michael J. Taussig, 2002. Ribosome display: Cell-free protein display technology. Briefings in functional genomics and proteomics. Vol 1: 204-212; Patrick Dufner et al., 2006. Harnessing phage and ribosome display for antibody optimization. Trends in Biotechnology, Vol. 24: 523-529; each of which is fully incorporated herein by reference).

Peptibodies are isolated polypeptide comprising at least one peptide capable of binding to an antigen (e.g., a CDR) attached to an Fc domain of an antibody (e.g., IgG, IgA, IgD, IgE, IgM antibodies) or a fragment of an Fc domain. A peptibody can include more than one peptide capable of binding an antigen (e.g., 2, 3, 4 or 5 peptides) which may be the same as one another or may be different from one another.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; (5) Single chain antibody (“SCA”), a genetically engineered molecule containing the variable region of the light chain and the variable region of the heavy chain, linked by a suitable polypeptide linker as a genetically fused single chain molecule; (6) CDR peptide is a peptide coding for a single complementarity-determining region (CDR); and (7) Single domain antibodies (also called nanobodies), a genetically engineered single monomeric variable antibody domain which selectively binds to a specific antigen. Nanobodies have a molecular weight of only 12-15 kDa, which is much smaller than a common antibody (150-160 kDa).

According to some embodiments of the invention, the antigen binding domain comprises complementarity determining region (CDR) selected from the group of the CDRs set forth by SEQ ID NOs:171-173 CDRs 1-3 for light chain; SEQ ID NOs:177-179 CDRs 1-3 for heavy chain, and 183-185 CDRs 1-3 for light chain; SEQ ID NOs:189-191 CDRs 1-3 for heavy chain.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

According to some embodiments of the invention, the antibodies are multivalent forms such as tetrameric Fabs, IgM or IgG1 antibodies, thus forming a multivalent composition with higher avidity to the target.

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including screening of phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

For in vivo use (for administering in a subject, e.g., human), the human or humanized antibody will generally tend to be better tolerated immunologically than one of non human origin since non variable portions of non human antibodies will tend to trigger xenogeneic immune responses more potent than the allogeneic immune responses triggered by human antibodies which will typically be allogeneic with the individual. It will be preferable to minimize such immune responses since these will tend to shorten the half-life, and hence the effectiveness, of the antibody in the individual. Furthermore, such immune responses may be pathogenic to the individual, for example by triggering harmful inflammatory reactions.

Alternately, an antibody of a human origin, or a humanized antibody, will also be advantageous for applications (such as targeted cell killing) in which a functional physiological effect, for example an immune response against a target cell, activated by a constant region of the antibody in the individual is desired. In these cases, an optimal functional interaction occurs when the functional portion of the antibody, such as the Fc region, and the molecule interacting therewith such as the Fc receptor or the Fc-binding complement component are of a similar origin (e.g., human origin).

Depending on the application and purpose, the antibody of the invention, which includes a constant region, or a portion thereof of any of various isotypes, may be employed. According to some embodiments of the invention, the isotype is selected so as to enable or inhibit a desired physiological effect, or to inhibit an undesired specific binding of the antibody via the constant region or portion thereof. For example, for inducing antibody-dependent cell mediated cytotoxicity (ADCC) by a natural killer (NK) cell, the isotype can be IgG; for inducing ADCC by a mast cell/basophil, the isotype can be IgE; and for inducing ADCC by an eosinophil, the isotype can be IgE or IgA. For inducing a complement cascade the antibody may comprise a constant region or portion thereof capable of initiating the cascade. For example, the antibody may advantageously comprise a Cgamma2 domain of IgG or Cmu3 domain of IgM to trigger a Clq-mediated complement cascade.

Conversely, for avoiding an immune response, such as the aforementioned one, or for avoiding a specific binding via the constant region or portion thereof, the antibody of the invention may not comprise a constant region (be devoid of a constant region), a portion thereof or specific glycosylation moieties (required for complement activation) of the relevant isotype.

According to an aspect of some embodiments of the invention, there is provided an isolated antibody comprising an antigen binding domain capable of specifically binding the isolated complex of MHC class II-GAD antigenic peptide of some embodiments of the invention. The isolated antibody does not bind to the MHC class II in an absence of the antigenic peptide, wherein the isolated antibody does not bind the antigenic peptide in an absence of the MHC class II.

According to some embodiments of the invention the antibody of some embodiments of the invention binds to the target complex (MHC class II-GAD autoantigen) with an affinity characterized by a dissociation constant which is lower than about 100 nanomolar, e.g., lower than about 50 nanomolar, e.g., lower than about 20 nanomolar, e.g., about 10 nanomolar or lower.

Once the CDRs of an antibody are identified, using conventional genetic engineering techniques, expressible polynucleotides encoding any of the forms or fragments of antibodies described herein can be synthesized and modified in one of many ways in order to produce a spectrum of related-products.

For example, to generate the high affinity entity of the invention (e.g., the antibody of the invention), an isolated polynucleotide sequence [e.g., SEQ ID NOs:174 (CDR1 of the G3H8 Ab light chain), 175 (CDR2 of the G3H8 Ab light chain), 176 (CDR3 of the G3H8 Ab light chain), 180 (CDR1 of the G3H8 Ab heavy chain), 181 (CDR2 of the G3H8 Ab heavy chain), 182 (CDR3 of the G3H8 Ab heavy chain), 159 (nucleic acid sequence encoding the G3H8 Ab light chain) or 161 (nucleic acid sequence encoding the G3H8 Ab heavy chain] encoding the amino acid sequence of the antibody of the invention [e.g., SEQ ID NOs:171 (CDR1 of the G3H8 Ab light chain), 172 (CDR2 of the G3H8 Ab light chain), 173 (CDR3 of the G3H8 Ab light chain), 177 (CDR1 of the G3H8 Ab heavy chain), 178 (CDR2 of the G3H8 Ab heavy chain), 189 (CDR3 of the G3H8 Ab heavy chain), 158 (amino acid sequence of the G3H8 Ab light chain) or 160 (amino acid sequence of the G3H8 Ab heavy chain)] is preferably ligated into a nucleic acid construct (expression vector) suitable for expression in a host cell. Such a nucleic acid construct includes a promoter sequence for directing transcription of the polynucleotide sequence in the cell in a constitutive or inducible manner.

The nucleic acid construct of the invention may also include an enhancer, a transcription and translation initiation sequence, transcription and translation terminator and a polyadenylation signal, a 5′ LTR, a tRNA binding site, a packaging signal, an origin of second-strand DNA synthesis, and a 3′ LTR or a portion thereof; a signal sequence for secretion of the antibody polypeptide from a host cell; additional polynucleotide sequences that allow, for example, the translation of several proteins from a single mRNA such as an internal ribosome entry site (IRES) and sequences for genomic integration of the promoter-chimeric polypeptide; sequences engineered to enhance stability, production, purification, yield or toxicity of the expressed peptide.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p205. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Various methods can be used to introduce the nucleic acid construct of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Recombinant viral vectors are useful for in vivo expression since they offer advantages such as lateral infection and targeting specificity. Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses.

As mentioned hereinabove, a variety of prokaryotic or eukaryotic cells can be used as host-expression systems to express the antibody of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the antibody of the invention.

Recovery of the recombinant antibody polypeptide is effected following an appropriate time in culture. The phrase “recovering the recombinant polypeptide” refers to collecting the whole fermentation medium containing the polypeptide and need not imply additional steps of separation or purification. Not withstanding the above, antibody polypeptides of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

According to an aspect of some embodiments of the invention, there is provided a molecule comprising the high affinity entity (e.g., the antibody) of the invention being conjugated to a functional moiety (also referred to as an “immunoconjugate”) such as a detectable or a therapeutic moiety. The immunoconjugate molecule can be an isolated molecule such as a soluble or synthetic molecule.

Various types of detectable or reporter moieties may be conjugated to the high affinity entity of the invention (e.g., the antibody of the invention). These include, but not are limited to, a radioactive isotope (such as ^{[125]}iodine), a phosphorescent chemical, a chemiluminescent chemical, a fluorescent chemical (fluorophore), an enzyme, a fluorescent polypeptide, an affinity tag, and molecules (contrast agents) detectable by Positron Emission Tomagraphy (PET) or Magnetic Resonance Imaging (MRI).

Examples of suitable fluorophores include, but are not limited to, phycoerythrin (PE), fluorescein isothiocyanate (FITC), Cy-chrome, rhodamine, green fluorescent protein (GFP), blue fluorescent protein (BFP), Texas red, PE-Cy5, and the like. For additional guidance regarding fluorophore selection, methods of linking fluorophores to various types of molecules see Richard P. Haugland, “Molecular Probes: Handbook of Fluorescent Probes and Research Chemicals 1992-1994”, 5th ed., Molecular Probes, Inc. (1994); U.S. Pat. No. 6,037,137 to Oncoimmunin Inc.; Hermanson, “Bioconjugate Techniques”, Academic Press New York, N.Y. (1995); Kay M. et al., 1995. Biochemistry 34:293; Stubbs et al., 1996. Biochemistry 35:937; Gakamsky D. et al., “Evaluating Receptor Stoichiometry by Fluorescence Resonance Energy Transfer,” in “Receptors: A Practical Approach,” 2nd ed., Stanford C. and Horton R. (eds.), Oxford University Press, UK. (2001); U.S. Pat. No. 6,350,466 to Targesome, Inc.]. Fluorescence detection methods which can be used to detect the high affinity entity (e.g., antibody) when conjugated to a fluorescent detectable moiety include, for example, fluorescence activated flow cytometry (FACS), immunofluorescence confocal microscopy, fluorescence in-situ hybridization (FISH) and fluorescence resonance energy transfer (FRET).

Numerous types of enzymes may be attached to the high affinity entity (e.g., the antibody) of some embodiments of the invention [e.g., horseradish peroxidase (HPR), beta-galactosidase, and alkaline phosphatase (AP)] and detection of enzyme-conjugated antibodies can be performed using ELISA (e.g., in solution), enzyme-linked immunohistochemical assay (e.g., in a fixed tissue), enzyme-linked chemiluminescence assay (e.g., in an electrophoretically separated protein mixture) or other methods known in the art [see e.g., Khatkhatay M I. and Desai M., 1999. J Immunoassay 20:151-83; Wisdom G B., 1994. Methods Mol. Biol. 32:433-40; Ishikawa E. et al., 1983. J Immunoassay 4:209-327; Oellerich M., 1980. J Clin Chem Clin Biochem. 18:197-208; Schuurs A H. and van Weemen B K., 1980. J Immunoassay 1:229-49).

The affinity tag (or a member of a binding pair) can be an antigen identifiable by a corresponding antibody [e.g., digoxigenin (DIG) which is identified by an anti-DIG antibody) or a molecule having a high affinity towards the tag [e.g., streptavidin and biotin]. The antibody or the molecule which binds the affinity tag can be fluorescently labeled or conjugated to enzyme as described above.

Various methods, widely practiced in the art, may be employed to attach a streptavidin or biotin molecule to the antibody of the invention. For example, a biotin molecule may be attached to the antibody of the invention via the recognition sequence of a biotin protein ligase (e.g., BirA) as described in the Examples section which follows and in Denkberg, G. et al., 2000. Eur. J. Immunol. 30:3522-3532. Alternatively, a streptavidin molecule may be attached to an antibody fragment, such as a single chain Fv, essentially as described in Cloutier S M. et al., 2000. Molecular Immunology 37:1067-1077; Dubel S. et al., 1995. J Immunol Methods 178:201; Huston J S. et al., 1991. Methods in Enzymology 203:46; Kipriyanov S M. et al., 1995. Hum Antibodies Hybridomas 6:93; Kipriyanov S M. et al., 1996. Protein Engineering 9:203; Pearce L A. et al., 1997. Biochem Molec Biol Intl 42:1179-1188).

Functional moieties, such as fluorophores, conjugated to streptavidin are commercially available from essentially all major suppliers of immunofluorescence flow cytometry reagents (for example, Pharmingen or Becton-Dickinson).

According to some embodiments of the invention, biotin conjugated antibodies are bound to a streptavidin molecule to form a multivalent composition (e.g., a dimer or tetramer form of the antibody).

Table 1 provides non-limiting examples of identifiable moieties which can be conjugated to the antibody of the invention.

TABLE 1
Table 1.

| | Amino Acid sequence | Nucleic Acid sequence |
|---|---|---|
| Identifiable | (GenBank Accession No.)/ | (GenBank Accession |
| Moiety | SEQ ID NO: | No.)/SEQ ID NO: |
| Green Fluorescent | AAL33912/225 | AF435427/226 |
| protein | | |
| Alkaline | AAK73766/227 | AY042185/228 |
| phosphatase | | |
| Peroxidase | CAA00083/229 | A00740/230 |
| Histidine tag | Amino acids 264-269 of | Nucleotides 790-807 of |
| | GenBank Accession No. | GenBank Accession No. |
| | AAK09208/231 | AF329457/232 |
| Myc tag | Amino acids 273-283 of | Nucleotides 817-849 of |
| | GenBank Accession No. | GenBank Accession No. |
| | AAK09208/231 | AF329457/232 |
| Biotin lygase tag | LHHILDAQKMVWNHR/ | |
| | 259 | |
| orange | AAL33917/235 | AF435432/236 |
| fluorescent | | |
| protein | | |
| Beta | ACH42114/237 | EU626139/238 |
| galactosidase | | |
| Streptavidin | AAM49066/239 | AF283893/240 |

As mentioned, the high affinity entity (e.g., the antibody) may be conjugated to a therapeutic moiety. The therapeutic moiety can be, for example, a cytotoxic moiety, a toxic moiety, a cytokine moiety and a second antibody moiety comprising a different specificity to the antibodies of the invention.

Non-limiting examples of therapeutic moieties which can be conjugated to the high affinity entity (e.g., the antibody) of the invention are provided in Table 2, hereinbelow.

TABLE 2
Table 2.

| | Amino acid sequence | Nucleic acid sequence |
|---|---|---|
| | (GenBank Accession | (GenBank Accession |
| Therapeutic moiety | No.)/SEQ ID NO: | No.)/SEQ ID NO: |
| Pseudomonas exotoxin | ABU63124/241 | EU090068/242 |
| Diphtheria toxin | AAV70486/243 | AY820132.1/244 |
| interleukin 2 | CAA00227/245 | A02159/246 |
| CD3 | P07766/247 | X03884/248 |
| CD16 | NP_000560.5/249 | NM_000569.6/250 |
| interleukin 4 | NP_000580.1/251 | NM_000589.2/252 |
| HLA-A2 | P01892/253 | K02883/254 |
| interleukin 10 | P22301/255 | M57627/256 |
| Ricin toxin | EEF27734/257 | EQ975183/258 |

According to some embodiments of the invention, the toxic moiety is PE38 KDEL [(SEQ ID NO:233 for protein) and SEQ ID NO:234 for nucleic acid).

The functional moiety (the detectable or therapeutic moiety of the invention) may be attached or conjugated to the high affinity entity (e.g., the antibody) of the invention in various ways, depending on the context, application and purpose.

When the functional moiety is a polypeptide, the immunoconjugate may be produced by recombinant means. For example, the nucleic acid sequence encoding a toxin (e.g., PE38 KDEL) or a fluorescent protein [e.g., green fluorescent protein (GFP), red fluorescent protein (RFP) or yellow fluorescent protein (YFP)] may be ligated in-frame with the nucleic acid sequence encoding the high affinity entity (e.g., the antibody) of the invention and be expressed in a host cell to produce a recombinant conjugated antibody. Alternatively, the functional moiety may be chemically synthesized by, for example, the stepwise addition of one or more amino acid residues in defined order such as solid phase peptide synthetic techniques.

A functional moiety may also be attached to the high affinity entity (e.g., the antibody) of the invention using standard chemical synthesis techniques widely practiced in the art [see e.g., hypertexttransferprotocol://worldwideweb (dot) chemistry (dot) org/portal/Chemistry)], such as using any suitable chemical linkage, direct or indirect, as via a peptide bond (when the functional moiety is a polypeptide), or via covalent bonding to an intervening linker element, such as a linker peptide or other chemical moiety, such as an organic polymer. Chimeric peptides may be linked via bonding at the carboxy (C) or amino (N) termini of the peptides, or via bonding to internal chemical groups such as straight, branched or cyclic side chains, internal carbon or nitrogen atoms, and the like. Description of fluorescent labeling of antibodies is provided in details in U.S. Pat. Nos. 3,940,475, 4,289,747, and 4,376,110.

Exemplary methods for conjugating peptide moieties (therapeutic or detectable moieties) to the high affinity entity (e.g., the antibody) of the invention are described herein below:

SPDP Conjugation—

A non-limiting example of a method of SPDP conjugation is described in Cumber et al. (1985, Methods of Enzymology 112: 207-224). Briefly, a peptide, such as a detectable or therapeutic moiety (e.g., 1.7 mg/ml) is mixed with a 10-fold excess of SPDP (50 mM in ethanol); the antibody is mixed with a 25-fold excess of SPDP in 20 mM sodium phosphate, 0.10 M NaCl pH 7.2 and each of the reactions is incubated for about 3 hours at room temperature. The reactions are then dialyzed against PBS. The peptide is reduced, e.g., with 50 mM DTT for 1 hour at room temperature. The reduced peptide is desalted by equilibration on G-25 column (up to 5% sample/column volume) with 50 mM KH₂PO₄ pH 6.5. The reduced peptide is combined with the SPDP-antibody in a molar ratio of 1:10 antibody:peptide and incubated at 4° C. overnight to form a peptide-antibody conjugate.

Glutaraldehyde conjugation—

A non-limiting example of a method of glutaraldehyde conjugation is described in G. T. Hermanson (1996, “Antibody Modification and Conjugation, in Bioconjugate Techniques, Academic Press, San Diego). Briefly, the antibody and the peptide (1.1 mg/ml) are mixed at a 10-fold excess with 0.05% glutaraldehyde in 0.1 M phosphate, 0.15 M NaCl pH 6.8, and allowed to react for 2 hours at room temperature. 0.01 M lysine can be added to block excess sites. After—the reaction, the excess glutaraldehyde is removed using a G-25 column equilibrated with PBS (10% v/v sample/column volumes)

Carbodiimide Conjugation—

Conjugation of a peptide with an antibody can be accomplished using a dehydrating agent such as a carbodiimide, e.g., in the presence of 4-dimethyl aminopyridine. Carbodiimide conjugation can be used to form a covalent bond between a carboxyl group of peptide and an hydroxyl group of an antibody (resulting in the formation of an ester bond), or an amino group of an antibody (resulting in the formation of an amide bond) or a sulfhydryl group of an antibody (resulting in the formation of a thioester bond). Likewise, carbodiimide coupling can be used to form analogous covalent bonds between a carbon group of an antibody and an hydroxyl, amino or sulfhydryl group of the peptide [see, J. March, Advanced Organic Chemistry: Reaction's, Mechanism, and Structure, pp. 349-50 & 372-74 (3d ed.), 1985]. For example, the peptide can be conjugated to an antibody via a covalent bond using a carbodiimide, such as dicyclohexylcarbodiimide [B. Neises et al. (1978), Angew Chem., Int. Ed. Engl. 17:522; A. Hassner et al. (1978, Tetrahedron Lett. 4475); E. P. Boden et al. (1986, J. Org. Chem. 50:2394) and L. J. Mathias (1979, Synthesis 561)].

As mentioned above and further illustrated in the Examples section which follows, the isolated high affinity entity (e.g., the antibody) according to some embodiments of the invention can be used to detect the complex of MHC class II and a diabetes associate autoantigen (e.g., the GAD autoantigenic peptide) on the surface antigen Presenting Cells (APC) such as dendritic cells, macrophages and B-cells.

Thus, according to an aspect of some embodiments of the invention, there is provided a method of detecting presentation of a type I diabetes-associated autoantigenic peptide on a cell. The method is effected by contacting the cell with the high affinity entity of some embodiments of the invention, the molecule of some embodiments of the invention, or the antibody of some embodiments of the invention, under conditions which allow immunocomplex formation, wherein a presence or a level above a predetermined threshold of the immunocomplex is indicative of presentation of the diabetes-associated autoantigenic peptide on the cell.

The cell presenting the diabetes-associated autoantigenic peptide (e.g., GAD antigen) can be any nucleated cell such as an antigen presenting cell (APC) in the blood, pancreas and lymphoid organs such as thymus, bone marrow, lymph node and lymphoid follicles.

Contacting the cell with the high affinity entity (e.g., the antibody)/molecule or multivalent composition of the invention may be effected in vitro (e.g., in a cell line), ex vivo or in vivo.

As mentioned, the method of the invention is effected under conditions sufficient to form an immunocomplex; such conditions (e.g., appropriate concentrations, buffers, temperatures, reaction times) as well as methods to optimize such conditions are known to those skilled in the art, and examples are disclosed herein.

As used herein the phrase “immunocomplex” refers to a complex which comprises the high affinity entity of some embodiments of the invention (e.g., the antibody) and the MHC-class II-diabetes-associated autoantigenic peptide (e.g., GAD peptide). Determining a presence or level of the immunocomplex of the invention is performed using the detectable moiety to which the high affinity entity (e.g., antibody) is attached, and can be performed using various methods are known in the art and described hereinabove.

The level of the immunocomplex in the tested cell (e.g., a cell of a subject in need thereof) is compared to a predetermined threshold. The threshold may be determined based on a known reference level and/or a level in a control cell. The control cell can be obtained from a control, healthy subject (e.g., a subject not diagnosed with diabetes or not being at-risk for diabetes, or from a subject devoid of the specific MHC molecule forming the MHC-peptide complex (e.g., DR4). According to some embodiments of the invention, the control subject is of the same species e.g. human, preferably matched with the same age, weight, sex etc. as the subject in need thereof.

Thus, the teachings of the invention can be used to detect cells which present diabetes-associated autoantigenic peptideic peptides (e.g., GAD presenting cell(s)) in a biological sample of the subject.

As used herein the phrase “cells which present diabetes-associated autoantigenic peptides” refers to any cell or a portion thereof of the subject which displays the complex of MHC class II and MHC-restricted diabetes-associated autoantigenic peptide.

The biological sample can be any sample which contains cells or a portion thereof (e.g., cell debris, membrane vesicles) which putatively present the MHC class II-diabetes-associated autoantigenic peptide complex.

According to some embodiments of the invention, the subject is at risk of developing type 1 diabetes. Non-limiting examples of subjects who are at risk to develop type 1 diabetes include subjects carrying the HLA DRB1*03,*04; DQB1*0302 genotype and the DR3-DQ2 and DR4-DQ8 haplotypes.

Type 1 diabetes results from autoimmune destruction of insulin-producing beta cells of the pancreas, which lead to lack of insulin and subsequently increased blood and urine glucose. Classical symptoms include polyuria (frequent urination), polydipsia (increased thirst), polyphagia (increased hunger), and weight loss.

To date, the diagnosis of type 1 diabetes is made by demonstrating any one of the following: Fasting plasma glucose level at or above 7.0 mmol/L (126 mg/dL); Plasma glucose at or above 11.1 mmol/L (200 mg/dL) two hours after a 75 g oral glucose load as in a glucose tolerance test; Symptoms of hyperglycemia and casual plasma glucose at or above 11.1 mmol/L (200 mg/dL); Glycated hemoglobin (hemoglobin A1C) at or above 6.5. Thus, in most cases, when type 1 diabetes is diagnosed most of the beta cells in the pancreas are destroyed.

Early signs of type 1 diabetes include the development of islets autoantibodies. Autoantibodies to four islet antigen groups have so far been identified: insulin or proinsulin, GAD65 or GAD67, IA-2 (PHOGRIN), and ZnT8. The number of islets autoantibodies, greater titer, affinity, and broadness of epitope reactivity are features of—autoantibodies that affect the risk for T1D. Combination of family history information, genetic factors, autoantibodies, age and beta cells function markers provides a disease risk determination that can be calculated empirically.

As shown in Example 4 of the Examples section, the isolated antibodies of some embodiments of the invention were shown capable of detecting APC (which present the MHC class II-GAD antigenic peptide) in the infiltrated islets of diabetic B7/DR4 mice. Moreover, the isolated antibodies of some embodiments of the invention were shown capable of detecting APC in the infiltrated islets of pre-diabetic young B7/DR4 mice, thus diagnosing early signs of beta cell destruction leading to type 1 diabetes.

Using the currently available diagnostic tools, at the time a diagnosis of type I diabetes is made in a subject about 90% of the insulin producing cells are destroyed (Gepts W. Pathologic anatomy of pancreas in juvenile diabetes mellitus. Diabetes 1965; 14: 619-633).

It should be noted that diagnosing type 1 diabetes at the early stages of the disease is of significant importance since not all of the beta cells in the pancreas are destroyed. Thus, early detection of type 1 diabetes, before a complete diagnosis is made, is of great significance, since it enables clinical intervention and treatment which will prevent the complete destruction of beta cells.

Antigen-specific tolerance approaches are desirable treatment of T1D. The focus of these developing treatment strategies is to safely inactivate pathogenic autoreactive T cells in an autoantigen-specific manner while leaving the remainder of immune system unperturbed. Identification of the antigen-specificity nature of the immune response prior to antigen-specific intervention will allow the adjustment of the suitable treatment for the current auto-immune response of the subject. The isolated antibodies of some embodiments of the invention were shown capable of detecting specific auto-antigens presentation, and therefore identifying the specific-antigenic nature of the auto-immune process. Thus, the teachings of the invention can be used to select an accurate and most suitable antigen-specific intervention strategy.

Thus, according to an aspect of some embodiments of the invention, there is provided a method of diagnosing type 1 diabetes (T1D) in a subject. The method is effected by contacting a cell of the subject with the high affinity entity (e.g., antibody) of some embodiments of the invention, the molecule of some embodiments of the invention, or the multivalent antibody of some embodiments of the invention under conditions which allow immunocomplex formation, wherein a presence or a level above a pre-determined threshold of the immunocomplex in the cell is indicative of the type 1 diabetes in the subject.

As used herein the term “diagnosing” refers to determining presence or absence of a pathology, classifying a pathology or a symptom, determining a severity of the pathology, monitoring pathology progression, forecasting an outcome of a pathology and/or prospects of recovery.

According to some embodiments of the invention, diagnosis of type 1 diabetes relates to detecting early signs of the disease, even before the destruction of beta cells has began and the beta cells are still functional (i.e., produce insulin in response to elevation in glucose levels).

To facilitate diagnosis, the above teachings can be combined with other methods of diagnosing type 1 diabetes which are well known in the art.

Since as shown by the present inventors presentation of the MHC class II-GAD antigenic peptide complex by APCs (Dendritic cells, macrophages etc.) in the infiltrated islets begins at early stages of the disease, antibodies which specifically bind to cells presenting the complex of MHC class II and a diabetes-associated autoantigenic peptide can be used to treat type 1 diabetes.

Thus, according to an aspect of some embodiments of the invention, there is provided a method of treating type 1 diabetes (T1D), comprising administering to a subject in need thereof a therapeutically effective amount of the isolated high affinity entity (e.g., antibody) of some embodiments of the invention, the molecule of some embodiments of the invention (e.g., which includes the high affinity entity conjugated to a therapeutic moiety such as toxin), the multivalent composition comprising same of some embodiments of the invention, the isolated polynucleotide or the nucleic acid construct encoding same, thereby treating the treating type 1 diabetes (T1D).

The term “treating” refers to inhibiting or arresting the development of a disease, disorder or condition and/or causing the reduction, remission, or regression of a disease, disorder or condition. Those of skill in the art will understand that various methodologies and assays can be used to assess the development of a disease, disorder or condition, and similarly, various methodologies and assays may be used to assess the reduction, remission or regression of a disease, disorder or condition.

According to some embodiments of the invention, treatment of type 1 diabetes is achieved by blocking presentation of the MHC class II/diabetes-associated autoantigenic peptide on APCs, and thus preventing or avoiding recognition of the antigen presenting cells by the specific T cells.

It should be noted that by blocking the presentation of the MHC class II-antigenic peptide complex by APCs, the inflammatory process and reactions that are induced by these APCs are also blocked, thereby reducing and eliminating the destruction of the beta cells in the islets that produce insulin.

According to some embodiments of the invention, treatment with the isolated antibodies of the invention is performed at an early stage of disease, before the onset of diabetic symptoms.

According to some embodiments of the invention, treatment with the isolated high affinity entity (e.g., the antibody) of some embodiments of the invention prevents the symptoms of glucose blood level increase and the subsequent need for insulin administration (e.g., by injections) because the beta cell own insulin production is spared.

According to some embodiments of the invention, for the inhibition approach, i.e., inhibition of MHC class II-type I diabetes-associate autoantigen presentation on APC (e.g., MHC class II-GAD antigen presentation on APCs) the effector functions of the high affinity entity (e.g., antibody) are manipulated such that the high affinity entity (e.g., antibody) is devoid of an Antibody-Dependent Cell-Mediated Cytotoxicity (ADCC) activity or devoid of a Complement-Dependent Cytotoxicity (CDC) activity. For example, the antibody of some embodiments of the invention is devoid of a constant region, a portion thereof or specific glycosylation moieties (required for complement activation) of the relevant isotype.

Additionally or alternatively, the high affinity entity (e.g., antibody) of the invention can be used to directly kill the APCs which display the diabetes-associated autoantigenic peptide (e.g., GAD antigenic peptide) in a complex with the MHC class II.

According to some embodiments of the invention, for the killing approach (i.e., killing of APCs which present the complex of MHC class II and diabetes-associated autoantigenic peptide), the isolated high affinity entity (e.g., antibody) is a naked high affinity entity that is capable of mediating ADCC or CDC.

As used herein the term “naked” refers to being devoid of a conjugated moiety such as a detectable or a therapeutic moiety.

According to some embodiments of the naked antibody comprises the constant region, a portion thereof or specific glycosylation moieties which mediate ADCC or CDC.

According to some embodiments of the invention, for the killing approach (i.e., killing of APCs which present the MHC class II-diabetes-associated autoantigenic peptide, the isolated high affinity entity (e.g., the antibody) is conjugated to a therapeutic moiety (e.g., drug, toxic moiety) that will kill the APCs presenting the MHC class II-GAD antigenic complex.

According to some embodiments of the invention, for the drug is an anti-inflammatory drugs or a cytokine that will reduce or inhibit the local inflammation in the islets and thus will rescue and inhibit the damage to the insulin producing beta cells.

According to some embodiments of the invention, the isolated high affinity entity (e.g., the antibody), molecule comprising same, multivalent antibody composition, polynucleotide, and/or nucleic acid construct of the invention is capable of killing MHC class II-diabetes-associated autoantigenic peptides (e.g., GAD) presenting cells in the subject in need thereof.

The high affinity entity (e.g., the antibody) of the invention, the molecule of the invention (which comprise the high affinity entity, e.g., antibody, conjugated to a therapeutic or detectable moiety), the multivalent composition of the invention, the isolated polynucleotide or the nucleic acid construct of the invention may be provided per se or may be administered as a pharmaceutical composition.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the high affinity entity (e.g., the antibody) of the invention, the molecule of the invention (which comprise the high affinity entity, e.g., an antibody, conjugated to a therapeutic or detectable moiety, or a polynucleotide encoding same), the multivalent composition of the invention, the isolated polynucleotide or the nucleic acid construct of the invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran.

Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the invention may also be formulated in rectal compositions such as suppositories or retention enemas, using, e.g., conventional suppository bases such as cocoa butter or other glycerides.

Pharmaceutical compositions suitable for use in context of the invention include compositions wherein the active ingredients are contained in an amount effective to achieve the intended purpose. More specifically, a therapeutically effective amount means an amount of active ingredients [e.g., the high affinity entity of the invention, e.g., the antibody of the invention, the molecule of the invention (e.g., which comprise the antibody conjugated to a therapeutic or detectable moiety), the multivalent composition of the invention, the isolated polynucleotide or the nucleic acid construct of the invention] effective to prevent, alleviate or ameliorate symptoms of a disorder (type 1 diabetes) or prolong the survival of the subject being treated.

Determination of a therapeutically effective amount is well within the capability of those skilled in the art, especially in light of the detailed disclosure provided herein.

For example, the effect of the active ingredients (e.g., the high affinity entity, e.g., the antibody of the invention, or the polynucleotide encoding same) on type 1 diabetes treatment can be evaluated by monitoring the level of glucose in the blood of the treated subject, and/or measuring the level of hemoglobin A1c using well known methods.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

According to some embodiments of the invention, the therapeutic agent of the invention (e.g., the high affinity entity of the invention, e.g., the antibody, molecule and/or multivalent composition of the invention) can be provided to the subject in combination with other drug(s) designed for treating type 1 diabetes (combination therapy). Non-limiting examples of such drugs include insulin (e.g., a recombinant human insulin, pig derived insulin) and Anti-CD3 mAb. Methods of administering insulin include injection, insulin pumps and inhaled insulin have been available at various times. Pancreas transplants have been also used to treat type 1 diabetes. The combination therapy may increase the therapeutic effect of the agent of the invention in the treated subject.

Compositions of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

The agents of some embodiments of the invention which are described hereinabove for detecting the complexes of MHC class II/diabetes-associated autoantigenic peptides (e.g., GAD antigenic peptide) (either in an isolated form or when displayed on cells) may be included in a diagnostic kit/article of manufacture preferably along with appropriate instructions for use and labels indicating FDA approval for use in diagnosing, determining predisposition to, and/or assessing type 1 diabetes.

Such a kit can include, for example, at least one container including at least one of the above described diagnostic agents (e.g., the high affinity entity, e.g., the antibody) and an imaging reagent packed in another container (e.g., enzymes, secondary antibodies, buffers, chromogenic substrates, fluorogenic material). The kit may also include appropriate buffers and preservatives for improving the shelf-life of the kit.

According to an aspect of some embodiments of the invention, there is provided a method of isolating a high affinity entity which specifically binds to a complex composed of a major histocompatibility complex (MHC) class II and a type I diabetes-associated autoantigenic peptide, comprising:

(a) screening a library comprising a plurality of high affinity entities with the isolated complex of some embodiments of the invention; and

(b) isolating at least one high affinity entity which specifically binds to the isolated complex of some embodiments of the invention and not to the MHC class II in the absence of the type I diabetes-associated autoantigenic peptide or to the type I diabetes-associated autoantigenic peptide in an absence of the MHC class II,

thereby isolating the high affinity entities which specifically bind to the complex of the MHC class II and the type I diabetes-associated autoantigenic peptide.

According to some embodiments of the invention, the high affinity entity further specifically binds to a native conformation of the complex of the MHC class II and the type I diabetes-associated autoantigenic peptide.

According to an aspect of some embodiments of the invention there is provided a composition of matter comprising the isolated MHC class II and diabetes-associated autoantigenic peptide complex of some embodiments of the invention and a conjugated functional moiety.

The conjugated functional moiety can be a therapeutic or a detectable moiety as described above. Conjugation of the functional moiety can be performed as described above and/or in U.S. Patent Application No. 20030166277 which is fully incorporated herein by reference.

According to some embodiments of the invention, the functional moiety comprises an antibody or a fragment specific for a cell surface marker. The cell surface marker can be expressed on an antigen presenting cell.

Examples of cell surface markers include, but are not limited to cell surface markers of tumor cells, epithelial cells, fibroblast, and T cells (e.g., CD28, CTLA-4 and CD25).

According to some embodiments of the invention, the functional moiety comprises a therapeutic moiety such as a cytokine or lymphokine. The cytokine or lymphokine may be linked to the MHC class II and diabetes-associated autoantigenic peptide complex either directly or via, e.g., formation of a multivalent compound (using streptavidin or avidin for example, and a biotinylated cytokine or lymphokine.

Non-limiting examples of cytokines or lymphokines include interleukins (e.g., IL-2, IL-3, IL-4, IL-5, IL-6, IL-10, IL-12, IL-15, and IL-18), alpha interferons (e.g., IFN.alpha.), beta interferons (e.g., IFN.beta.), gamma. interferons (e.g., IFN.gamma.), granulocyte-macrophage colony stimulating factor (GM-CSF), and transforming growth factor (TGF, e.g., TGF-alpha. and TGF-beta).

According to an aspect of some embodiments of the invention there is provided a pharmaceutical composition comprising the composition of matter of some embodiments of the invention and a therapeutically acceptable carrier as described above.

The composition of matter of some embodiments of the invention (e.g., which comprise the MHC class II/peptide complex conjugated to the functional moiety) is useful for modulating, i.e., either inhibiting or stimulating, an immune response; for stimulating desirable immune responses, for example, immune responses against infectious agents or cancer; for inhibiting undesirable immune responses, such as allergic responses, allograft rejections, and autoimmune diseases; by directing the MHC class II/diabetes-associated autoantigenic peptide complex to professional antigen presenting cells, such as dendritic cells, B cells, or macrophages; tumor cells; epithelial cells; fibroblasts; T cells; or other cells. Depending on the targeted cell type, this will lead to either very efficient stimulation or inhibition of antigen specific T cell activity.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader.

All the information contained therein is incorporated herein by reference.

### General Materials and Experimental Methods

Production of DR4 Molecules in S2 Cells—

DES TOPO DR-A1*0101/DR-B1*0401(HA-307-319) plasmids for inducible expression in Schneider S2 cell were used for cloning of DR-B1*0401(GAD₅₅₅₋₅₆₇) construct, transfection and expression of recombinant four-domain MHC class II as previously reported (Svendsen, P., et al., 2004). Briefly, in these constructs the intracellular domains of the DR-A and DR-B chains were replaced by leucine-zipper dimerization domains of Fos and Jun transcription factors, respectively, for heterodimer assembly. The antigenic peptide was introduced to the N-terminus of the DR-B chain through a flexible linker. Bir A recognition sequence for biotinylation was introduced to the C-terminus of the DR-A chain. DR-A and DR-B plasmids were co-transfected with pCoBlast selection vector to S2 cells using cellfectin reagent (invitrogen). Stable single-cell line clones were verified for protein expression. Upon induction with CuSO₄, cells supernatant were collected and DR4 complexes were affinity purified by anti-DR LB3.1 (ATCC number HB-298) monoclonal antibody (mAb). The purified DR4 complexes were biotinylated by Bir-A ligase (Avidity) and characterized by SDS-PAGE. The right folding of the complexes was verified by recognition of anti-DR conformation sensitive mAb (L243) in ELISA binding assay.

Selection of Phage Abs on Biotinylated Complexes—

Selection of phage Abs on biotinylated complexes was performed as described (Cohen C J et al., 2003, J Mol. Recognit. 2003, 16: 324-32). Briefly, a large human Fab library containing 3.7×10¹⁰ different Fab clones was used for the selection (de Haard H. J., et al., 1999). Phages were first preincubated with streptavidin-coated paramagnetic beads (200 μl; Dynal) to deplete the streptavidin binders. The remaining phages were subsequently used for panning with decreasing amounts of biotinylated MHC-peptide complexes. The streptavidin-depleted library was incubated in solution with soluble biotinylated DR4/GAD (500 nM for the first round, and 100 nM for the following rounds) for 30 minutes at room temperature. Streptavidin-coated magnetic beads (200 μl for the first round of selection and 100 μl for the following rounds) were added to the mixture and incubated for 10-15 minutes at room temperature. The beads were washed extensively 12 times with PBS/0.1% Tween 20 and an additional two washes were with PBS. Bound phages were eluted with triethylamine (100 mM, 5 minutes at room temperature), followed by neutralization with Tris-HCl (1 M, pH 7.4), and used to infect E. coli TG1 cells (OD=0.5) for 30 minutes at 37° C. The diversity of the selected Abs was determined by DNA fingerprinting using a restriction endonuclease (BstNI), which is a frequent cutter of Ab V gene sequences.

Expression and Purification of Soluble Recombinant Fab Abs—

TG1 or BL21 cells were grown to OD₆₀₀=0.8-1.0 and induced to express the recombinant Fab Ab by the addition of IPTG for 3-4 hours at 30° C. Periplasmic content was released using the B-PER solution (Pierce), which was applied onto a prewashed TALON column (Clontech). Bound Fabs were eluted using 0.5 ml of 100 mM in PBS. The eluted Fabs were dialyzed twice against PBS (overnight, 4° C.) to remove residual imidazole.

ELISA with Purified Fab Antibodies—

Binding specificity of individual soluble Fab fragments were determined by ELISA using biotinylated MHC/peptide complexes.

ELISA plates (Falcon) were coated overnight with BSA-biotin (1 μg/well). After being washed, the plates were incubated (1 hour at room temperature) with streptavidin (10 μg/ml), washed extensively, and further incubated (1 hour at room temperature) with 5 μg/ml of MHC/peptide complexes. The plates were blocked for 30 minutes at room temperature with PBS/2% skim milk and subsequently were incubated for 1 hour at room temperature with 5 μg/ml soluble purified Fab. After washing, plates were incubated with horseradish peroxidase-conjugated/anti-human-Fab antibody. Detection was performed using TMB reagent (Sigma).

Flow Cytometry—

DR4—EBV-transformed B lymphoblast Preisscells were incubated overnight with medium containing 70 μM with GAD₅₅₅₋₅₆₇ (NFFRMVISNPAAT; SEQ ID NO:12) or control peptide: GAD₅₅₂₋₅₇₂ (SEQ ID NO:203), HA₃₀₇₋₃₁₉ (PKYVKQNTLKLAT; SEQ ID NO:204), InsA₁₋₁₅ (GIVEQCCTSICSLYQ; SEQ ID NO: 205), and C11₂₆₁₋₂₇₃ (AGFKGEQGPKGEP; SEQ ID NO:206). GAD65 Altered Peptide Ligand (APL) that were loaded into Preiss were: M559Z (NFFRZVISNPAAT; SEQ ID NO:207), 1561M (NFFRMVMSNPAAT; SEQ ID NO:208), N563Q (NFFRMVISQPAAT; SEQ ID NO:209), 1561M-N563Q (NFFRMVMSQPAAT; SEQ ID NO:210). Cells (10⁶) were incubated with 1-5 μg of specific Fab for 1 hour at 4° C., followed by incubation with mouse-anti-myc Ab and FITC-labeled anti-mouse Ab for 45 minutes at 4° C. Cells were finally washed and analyzed by a FACSCalibur flow cytometer (BD).

IL-2 Bioassay for T Cell Hybridoma—

Hybridoma cells (10⁵/well in a 96-well plate) in 50 μl of 10% FBS-containing medium were combined with 50 μl 10⁵ irradiated (3000 rad) splenocytes of HLA-DRB1*0401-Tg mice and with 50 μl of 25 μg/ml individual peptides and various Fabs concentrations. The cells were incubated at 37° C. and 7% CO₂ for 24 hours. Supernatants were collected from the top of the culture for IL-2 capture ELISA.

Histology—

Fresh tissues were frozen in Tissue-Tek OTC compound (Sakura Finetek, Torrance, Calif. 9050) for immunofluorescence on frozen sections. Frozen sections (8 μm) were dried and blocked with 0.1% BSA/PBS for 30 minutes. G3H8 was added at 50 μg/ml for 1 hour at room temperature. Alexa-488-anti-human (A11013, Molecule probes, Eugene, Oreg., USA) was used as secondary Ab at 1:200 dilution. Fluorescence images were taken on Cell Observer—Zeiss Microscope.

### Example 1

### Isolation of Antibodies Specific to Dr4/Gad₅₅₅₋₅₆₇ Complex

For the isolation of TCRLs directed to the native MHC/peptide complexes the present inventors generated a recombinant DR4/GAD₅₅₅₋₅₆₇ complex which was used for screening of a phage display antibody library.

Recombinant DR4 Complexes—

Four-domain DR4 molecules were generated from a DR4 construct previously reported for expression in insect cells (Svendsen, P., et al., 2004) in which the intracellular domains of the DR-A1*0101 and DR-B1*0401 chains were replaced by leucine-zipper dimerization domains for heterodimer assembly (Svendsen, P., et al., 2004). The antigenic peptide was introduced to the N-terminus of the DR-B chain through a flexible linker. The Bir A recognition sequence for biotinylation was introduced to the C-terminus of the DR-A chain (FIG. 1A).

Screening of Ab Phage Display Library:

For selection of Fabs directed to DR4/GAD555-567 complex the present inventors screened a large Ab phage library, consisting of a repertoire of 3.7×10¹⁰ human recombinant Fab fragments (de Haard H. J., et al., 1999). For panning, biotinylated soluble DR4/GAD₅₅₅₋₅₆₇ complexes were used. Fab clones with peptide-dependent, MHC class II restricted specificity were of interest and were picked for further characterization. DNA fingerprinting by BstNI restriction reaction revealed 13 different restriction patterns of GAD peptide-dependent DR4 specific Fabs, indicating the selection of several different Fabs with such a unique specificity.

Specificity of TCR-like Fabs Toward DR4/GAD₅₅₅₋₅₆₇ Complexes:

The present inventors used E. coli cells to produce a soluble Fab form of a representative clone of each DNA restriction pattern. The specificity of the selected clones was characterized in ELISA binding assay (FIG. 2A). Four different TCRL Fab Abs (G1A1, G1H12, G3H8, G1A2) were isolated and found to bind solely to recombinant full length DR4/GAD₅₅₅₋₅₆₇ complexes and not to DR4 complexes with control peptides (i.e., the DR4 molecule without the GAD₅₅₅₋₅₆₇ peptide), or to the GAD₅₅₅₋₅₆₇ peptide alone. Additionally, these TCRLs successfully detect native DR4/GAD₅₅₅₋₅₆₇ complexes presented by EBV transformed DR4+ Priess B cell (FIG. 2B for representative G3H8Fab). In addition, the Fabs do not bind Preiss cells loaded with control DR4-associated peptides such as HA₃₀₇₋₃₁₆, InsA₁₋₁₅, CII₂₆₁₋₂₇₃ (FIG. 2B). GAD₅₅₅₋₅₆₇ is the minimal stimulating peptide within the GAD₅₅₂₋₅₇₂ naturally processed T cell epitope of the hGAD65 in the context of DR4 (Nepom G T, et al., 2001). Therefore, the present inventors tested the ability of the isolated TCRLs to recognize this naturally T1D-associated epitope. As seen in FIG. 2C, G3H8 binds Preiss cells loaded with GAD₅₅₂₋₅₇₂ with the same intensity as for the cells loaded with equal molar quantity of GAD₅₅₅₋₅₆₇ peptide. Same binding pattern obtained for all the selected DR4/GAD TCRL Fabs (data not shown). Further support for the TCR-like specificity characteristic of G3H8 came from the dose-depended binding to the DR4/GAD complexes on APCs as obtained from titrations of Fab concentrations (FIG. 2D) and loaded GAD₅₅₅₋₅₆₇ peptide concentration (FIG. 2E). Increasing in the percentages of DR4/GAD complexes within the total DR4/peptide complexes on the APCs found to be correlated with increased G3H8 staining intensity. In addition, this characterization of G3H8 and other TCRLs makes them suitable for quantification studies of specific MHC/peptide complexes presented by APC of interest.

### Example 2

### Fine Specificity of the G3H8 Antibody

Fine Specificity of G3H8 TCRL Fabs—

In order to localize the binding residues of the isolated TCRLs within the GAD peptide the present inventors tested the recognition of Preiss cells loaded with a set of hGAD65 altered peptide ligands (APL). A panel of peptides containing substitutions in the GAD65₅₅₅₋₅₆₇ sequence at TCR contact sites was used. Binding assays of G3H8 to DR4 complexes presenting GAD-555-567 peptides with amino acid substitutions M559Z (P3), 1561M (P5), N563Q (P7), or 1561M(P5)+N563Q(P7), located P5 as essential contact residue for G3H8-DR4/GAD555-567 interaction. TcR contact P5 position has been shown to be important for TcR5 interactions with this hGAD65 epitope (John A. et al., 2004), emphasizing the TCR-like nature of G3H8Fab. As shown in FIGS. 3A-F, Preiss cells loaded with GAD555-567 containing the single amino acid substitutions M559Z (FIG. 3B) and N563Q (FIG. 3D) obtained similar binding intensity of G3H8Fab as for Preiss cells loaded with the wild-type sequence of the GAD555-567 peptide (FIG. 3A). Contrary, Preiss cells loaded with GAD555-567 containing the single amino acid substitution 1561M (FIG. 3C) and the double amino acids substitution 1561M, N563Q (FIG. 3E) obtained significant decrease in the binding intensity of Fab G3H8 compared to the wild-type peptide. Thus, I561M substitution abolished the recognition of DR4/GAD555-567 complex by Fab G3H8 and highlighted position P5 as essential contact residue of G3H8 in the DR4/GAD555-567 complex. Since P5 is essential T-cell Receptor contact position of many known T cell clones specific to the DR4/GAD epitope, G3H8 potentially will able the inhibition of poly-clonal GAD-specific T cell response.

### Example 3

### The Isolated Antibodies of Some Embodiments of the Invention are Capable of Inhibiting Gad-Specific MHC Restricted T Cell Response

Blocking of GAD-specific DR0401 Restricted T Cell Response—

The present inventors further tested the ability of G3H8Fab to compete with the cognate TcR interaction with DR4/GAD complexes presented by APCs and by that to block this activating signal leading to T cells autoreactivity. The present inventors tested if G3H8 can inhibit Ag-specific activation of T cell hybridoma in a peptide-specific HLA-restricted manner. G3H8Fab found to inhibit ˜80% response of G2.1.36.1 T cell hybridoma specific to GAD-555-567 restricted by HLA-DR*0401 (FIG. 4A). Of important, G3H8 do not inhibit H1.13.2 hybridoma response to HA307-319 peptide restricted by HLA-DR*0401 (FIG. 4B). Thus, antigen-specific immunologic tolerance to the autoreactive GAD-epitope was in-vitro demonstrated by G3H8Fab.

### Example 4

### Identification of Antigen Presenting Cells which Present the GAD555-567 Peptide in Islets of Diabetic Transgenic Mice

Detection of DR4/GAD555-567 Complexes in Pancreas of Diabetic B7/0401 Tg-Mice—

RIP-B7 mice transgenic for the DR4 subtype DRA1*0101/B1*0401 were reported to develop spontaneous diabetes (Gebe J A, et al., 2006). Age-depended loss of cellular tolerance to the GAD₅₅₅₋₅₆₇ epitope (identical in all mouse and human isoforms) was identified in these mice, emphasizing their utility as humanized mice model mimicking the MHC-antigen interactions of the human disease. The present inventors used the G3H8Fab to test whether APC in the infiltrated islets of diabetic B7/DR0401 mice present the GAD₅₅₅₋₅₆₇ peptide on their MHC molecules. Positive staining of the G3H8 identified such complexes in islets of B7/DR4 diabetic mice (FIGS. 5A-C) and in infiltrated islets of B7/DR4 pre-diabetic mice (data not shown) as compared to islets from C57B6 control mice (FIGS. 5D-E). These results demonstrate the ability of G3H8Fab to detect and bind infiltrating APC presenting the beta cell-derived GAD555-567 autoantigen. G3H8Fab found to bind in a peptide-specific manner APC presenting GAD-autoantigen at the islets of langerhans of the pancreas. The demonstrated accessibility of G3H8 antibody to the islets infiltrating APC is essential for its therapeutic goal by blocking the down-stream activation of autoreactive T cells by these APC.

### Example 5

### Isolation of Specific Mhc Class II and Diabetes-Associated Autoantigenic Peptide Complexes

Tables 3, 4 and 5, hereinbelow, provides a list of MHC class II restricted diabetes associated autoantigens which can form a complex with MHC class II. Such complexes are used for isolation of specific antibodies useful for diagnostic and therapeutic purposes.

TABLE 3
Table 3. Provided are the diabetes-associated autoantigenic peptides (with their
sequence identifiers, SEQ ID NO:) and the MHC class II molecules which bind thereto.

| SEQ | | | SEQ | | | SEQ | | |
|---|---|---|---|---|---|---|---|---|
| ID | | | ID | | | ID | | |
| NO: | GAD | MHC | NO: | ZnT8 | MHC | NO: | IA-2 | MHC |
| 1 | MNILLQYV | DR4 | 46 | LTIQIESA | DQ8 | 54 | VSSVSSQFS | DR4 |
| | VKSFD | | | ADQDPS | | | DAAQASPS | |
| | | | | | | | SFSD | |
| 2 | IAPVFVLLE | DR4 | 47 | RTGIAQA | DQ8 | 55 | LAKEWQA | DR4 |
| | | | | LSSFDLH | | | LCAYQAEP | |
| | | | | | | | NTCATAQG | |
| | | | | | | | E | |
| 3 | LPRLIAFTSE | DR4 | 48 | LYPDYQI | DQ8 | 56 | KLKVESSP | DR4 |
| | HSHF | | | QAGIMIT | | | SRSDYINA | |
| | | | | | | | SPIIEHDP | |
| 4 | IAFTSEHSHF | DR4 | 49 | ILSVHVA | DQ8 | 57 | IKLKVESSP | DR4 |
| | SLK | | | TAASQDS | | | SRSDYINA | |
| | | | | | | | SPI | |
| 5 | TVYGAFDPL | DR4 | 50 | SKRLTFG | DQ8 | 58 | MVWESGC | DR4 |
| | LAVAD | | | WYRAEIL | | | TVIVMLTP | |
| | | | | | | | LVEDGV | |
| 6 | KYKIWMHV | DR4 | 51 | AILTDAA | DQ8 | 59 | RQHARQQ | DQ8 |
| | DAAWGGG | | | HLLIDLT | | | DKERLAAL | |
| | | | | | | | GPE | |
| 7 | KHKWKLSG | DR4 | 52 | KATGNRS | DQ8 | 60 | GPEGAHGD | DQ8 |
| | VERANSV | | | SKQAHA | | | TTFEYQDL | |
| | | | | K | | | CR | |
| 8 | LYNIIKNRE | DR4 | 53 | AVDGVIS | DQ8 | 61 | EGPPEPSR | DQ8 |
| | GYEMVF | | | VHSLHIW | | | VSSVSSQFS | |
| | | | | | | | D | |
| 9 | PSLRTLEDN | DR4 | | | | 62 | FSDAAQAS | DQ8 |
| | EERMSR | | | | | | PSSHSSTPS | |
| | | | | | | | W | |
| 10 | RMMEYGTT | DR4 | | | | 63 | AEPNTCAT | DQ8 |
| | MVSYQPL | | | | | | AQGEGNIK | |
| | | | | | | | KN | |
| 11 | SYQPLGDK | DR4 | | | | 64 | NASPIIEHD | DQ8 |
| | VNFFRMV | | | | | | PRMPAYIA | |
| | | | | | | | T | |
| 12 | NFFRMVISN | DR4 | | | | | DEGSALYH | DQ8 |
| | PAAT | | | | | 65 | VYEVNLVS | |
| | | | | | | | EH | |
| 13 | ATHQDIDFL | | | | | 66 | KGVKEIDI | DQ8 |
| | IEEIER | | | | | | AATLEHVR | |
| | | | | | | | DO | DR4 |
| 14 | ATDLLPACD | DQ8 | | | | 67 | FALTAVAE | DQ8 |
| | | | | | | | EVNAILKA | |
| | | | | | | | LPQ | |
| 15 | FDRSTKVID | DQ8 | | | | 68 | KNRSLAVL | DQ8 |
| | FHYPNE | | | | | | TYDHSRI | |
| 16 | ELLQEYNW | DQ8 | | | | 69 | GADPSADA | DQ8 |
| | E | | | | | | TEAYQEL | |
| 17 | EYNWELAD | DQ8 | | | | 70 | EIDIAATLE | DQ8 |
| | Q | | | | | | | |
| 18 | DIDFLIEEI | DQ8 | | | | 71 | NTCATAQG | DQ8 |
| | | | | | | | E | |
| 19 | TGHPRYFN | DQ8 | | | | 72 | EPNTCATA | DQ8 |
| | QLSTGLD | | | | | | Q | |
| 20 | TYEIAPVFV | DQ8 | | | | 73 | ERLAALGP | DQ8 |
| | LLEYVT | | | | | | E | |
| 21 | YVTLKKMR | DQ8 | | | | 74 | QHARQQD | DQ8 |
| | E | | | | | | KE | |
| 22 | PGGSGDGIF | DQ8 | | | | 75 | YEVNLVSE | DQ8 |
| | SPGGAISNM | | | | | | H | |
| | YA | | | | | | | |
| 23 | NMYAMMIA | DQ8 | | | | 76 | GASLYHVY | DQ8 |
| | RFKMFPEV | | | | | | E | |
| | KEKG | | | | | | | |
| 24 | PEVKEKGM | DQ8 | | | | 77 | FALTAVAE | DQ8 |
| | AALPRLIAF | | | | | | E | |
| | TSE | | | | | | | |
| 25 | DSVILIKCD | DQ8 | | | | 78 | GAHGDTTF | DQ8 |
| | | | | | | | E | |
| 26 | GKMIPSDLE | DQ8 | | | | 79 | GDTTFEYQ | DQ8 |
| | | | | | | | D | |
| 27 | ERRILEAKQ | DQ8 | | | | 80 | AAQASPSS | DQ8 |
| | | | | | | | H | |
| 28 | ERANSVTW | DQ8 | | | | 81 | SRVSSVSS | DQ8 |
| | N | | | | | | Q | |
| 29 | QCSALLVRE | DQ8 | | | | 82 | TQFHFLSW | DQ8 |
| | | | | | | | P | |
| 30 | KHYDLSYD | DQ8 | | | | 83 | EEPAQAN | DQ8 |
| | TGDKALQ | | | | | | MD | |
| 31 | AKGTTGFE | DQ8 | | | | 84 | GHMILAY | DQ8 |
| | AHVDKCL | | | | | | ME | |
| 32 | VDKCLELA | DQ8 | | | | 85 | MILAYMED | DQ8 |
| | EYLYNIIKN | | | | | | H | |
| | REG | | | | | | | |
| 33 | IIKNREGYE | DQ8 | | | | 86 | QALCAYQ | DQ8 |
| | | | | | | | AE | |
| 34 | MVFDGKPQ | DQ8 | | | | 87 | EWQALCA | DQ8 |
| | HTNVCFW | | | | | | YQ | |
| 35 | CFWYIPPSL | DQ8 | | | | 88 | LVRSKDQF | DQ8 |
| | RTLEDN | | | | | | E | |
| 36 | FWYIPPSLR | DQ8 | | | | 89 | VEDGVKQ | DQ8 |
| | TLED | | | | | | CD | |
| 37 | SLRTLEDNE | DQ8 | | | | 90 | YILIDMVL | DQ8 |
| | | | | | | | N | |
| 38 | ERMSRLSK | DQ8 | | | | 91 | ESGCTVIV | DQ8 |
| | VAPVIKA | | | | | | M | |
| 39 | IKARMMEY | DQ8 | | | | 92 | LCAYQAEP | DQ8 |
| | GTTMVSY | | | | | | N | |
| 40 | RMMEYGTT | DQ8 | | | | 93 | ETRTLTQF | DQ8 |
| | MVSYQPL | | | | | | H | |
| 41 | VISNPAATH | DQ8 | | | | 94 | VESSPSRSD | DQ8 |
| 42 | IDFLIEEIE | DQ8 | | | | 95 | GPLSHTIA | DQ8 |
| | | | | | | | D | |
| 43 | NWELADQP | DR2 | | | | 96 | SLFNRAEG | DQ8 |
| | QNLEEILMH | | | | | | P | |
| | CQT | | | | | | | |
| 44 | GHPRYFNQ | DR2 | | | | 97 | HPDFLPYD | DQ8 |
| | LSTG | | | | | | H | |
| 45 | TYEIAPVFV | DR2 | | | | 98 | HFLSWPAE | DQ8 |
| | LLFYVTLKK | | | | | | G | |
| | MR | | | | | | | |
| 267 | VNFFRMVIS | DR4 | | | | 99 | DFRRKVNK | DQ8 |
| | NPAATHQD | | | | | | C | |
| 268 | DKVNFFRM | DR4 | | | | 100 | HCSDGAGR | DQ8 |
| | VISNPAATH | | | | | | T | |
| | QDID | | | | | | | |
| 260 | FFRMVISNP | core | | | | 101 | LVRSFYLK | DQ8 |
| | A | sequence | | | | | N | |
| | | | | | | 102 | KNRSLAVL | DQ8 |
| | | | | | | | TYDHSRI | |
| | | | | | | 103 | GADPSADA | DQ8 |
| | | | | | | | TEAYQEL | |
| | | | | | | 104 | ANMDISTG | Unknown |
| | | | | | | | HMILAYME | |
| | | | | | | 105 | WQALCAY | unknown |
| | | | | | | | QAEPNTCA | |
| | | | | | | | T | |
| | | | | | | 106 | LSHTIADF | unknown |
| | | | | | | | WQMVWES | |
| | | | | | | | G | |
| | | | | | | 107 | DFWQMVW | unknown |
| | | | | | | | ESGCTVIV | |
| | | | | | | | M | |
| | | | | | | 108 | WESGCTVI | unknown |
| | | | | | | | VMLTPLVE | |
| | | | | | | 109 | VIVMLTPL | unknown |
| | | | | | | | VEDGVKQ | |
| | | | | | | | C | |
| | | | | | | 110 | SEHIWCED | unknown |
| | | | | | | | FLVRSFYL | |
| | | | | | | 111 | WCEDFLVR | unknown |
| | | | | | | | SFYLKNVQ | |
| | | | | | | 112 | EDFLVRSF | unknown |
| | | | | | | | YLKNVQT | |
| | | | | | | | Q | |
| | | | | | | 113 | DFRRKVNK | unknown |
| | | | | | | | CYRGRSCP | |
| | | | | | | 114 | YILIDMVL | unknown |
| | | | | | | | NRMAKGV | |
| | | | | | | | K | |
| | | | | | | 115 | FEFALTAV | unknown |
| | | | | | | | AEEVNAIL | |

TABLE 4
Table 4. Provided are the diabetes-associated
autoantigenic peptides (with their sequence
identifiers, SEQ ID NO:) and the MHC class
II molecules which bind thereto.

| SEQ | | | SEQ | |
|---|---|---|---|---|
| ID | | | ID | |
| NO: | PREPROINSULIN | MHC | NO: | HSP-60 |
| 116 | EALYLVCGE | DQ8 | 137 | KFGADARALMLQGVDLL |
| | | | | ADA |
| 117 | SICSLYQLE | | 138 | NPVEIRRGVMLAVDAVIA |
| | | | | EL |
| 118 | ALLALWGPD | | 139 | QSIVPALEIANAHRKPLVI |
| | | | | IA |
| 119 | GSLQPLALE | | 140 | LVLNRLKVGLQVVAVKA |
| | | | | PGF |
| 120 | TPKTRREAE | | 141 | IVLGGGCALLRCIPALDSL |
| | | | | T |
| 121 | PAAAFVNQH | | 142 | VLGGGCALLRCIPALDSL |
| | | | | TPANED |
| 122 | DPAAAFVNQ | | 143 | EIIKRTLKIPAMTIAKNAG |
| | | | | V |
| 123 | PDPAAAFVN | | 144 | VNMVEKGIIDPTKVVRTA |
| | | | | LL |
| 124 | QKRGIVEQC | | | |
| 125 | ELGGGPGAG | | | |
| 126 | EAEDLQVGQ | | | |
| 127 | LQVGQVELG | | | |
| 128 | HLCGSHLVE | | | |
| 129 | GIVEQCCTSICS | DR4 | | |
| 130 | KRGIVEQCCTSICS | | | |
| 131 | LALLALWGPDPAA | UNKNOWN | | |
| | AFV | | | |
| 132 | PAAAFVNQHLCGS | | | |
| | HLV | | | |
| 133 | SHLVEALYLVCGER | | | |
| | G | | | |
| 134 | FFYTPKTRREAED | | | |
| 135 | GAGSLQPLALEGSL | | | |
| | QKRG | | | |
| 136 | SLQKRGIVEQCCTSI | | | |
| | CS | | | |

TABLE 5
Table 5. Provided are the diabetes-associated
autoantigenic peptides (with their sequence
identifiers, SEQ ID NO:) and the MHC class
II molecules which bind thereto.

| SEQ | | SEQ | | |
|---|---|---|---|---|
| ID | | ID | | |
| NO: | HSP-70 | NO: | IGRP | MHC |
| 145 | MAKAAAVGIDLGTTYSCVG | 154 | QHLQKDYRAYYTF | DR3 |
| | V | | | |
| 146 | GLNVLRIINEPTAAAIAYGL | 155 | RVLNIDLLWSVPI | |
| 147 | TIDDGIFEVKATAGDTHLGG | | | |
| 148 | THLGGEDFDNRLVNHFVEEF | 156 | YTFLNFMSNVGDP | DR4 |
| 149 | KRTLSSSTQASLEIDSLFEG | 157 | DWIHIDTTPFAGL | |
| 150 | LLLLDVAPLSLGLETAGGV | | | |
| | M | | | |
| 151 | PTKQTQIFTTYSDNQPGVLI | | | |
| 152 | KANKITITNDKGRLSKEEIE | | | |
| 153 | KEEIERMVQEAEKYKAEDE | | | |
| | V | | | |

### Example 6

### Binding and Specificity of Whole IGG G3H8 Antibody

Experimental Results

Generation of G3H8 IgG Antibody—

The G3H8Fab was cloned into a fully human whole IgG molecule. The H and L Fab genes were cloned for expression as human IgG1 κ Ab into the eukaryotic expression vector pCMV/myc/ER. For the H chain, the multiple cloning site, the myc epitope tag, and the endoplasmic reticulum (ER) retention signal of pCMV/myc/ER were replaced by a cloning site containing recognition sites for BssHI and NheI followed by the human IgG1 constant H chain region cDNA isolated by RT-PCR from human lymphocyte total RNA. A similar construct was generated for the L chain. Each shuttle expression vector carries a different antibiotic resistance gene. Expression was facilitated by co-transfection of the two constructs into the human embryonic kidney HEK293 cell by using the FuGENE 6 Transfection Reagent (Roche). After co-transfection, cells were grown on selective medium. Clones that reacted specifically with Preiss cells pulsed with GAD-555-567 peptide were adapted to growth in 0.5% serum and were further purified using protein A affinity chromatography. SDS-PAGE analysis of the purified protein revealed homogenous, pure IgG with the expected molecular mass of 150 kDa.

Specificity of the G3H8 Antibody Towards Cells Presenting the HLA-DR4—GAD-555-567 Complexes Ex Vivo—

G3H8 TCRL specificity towards GAD antigen presenting cells (APCs) was demonstrated also ex vivo by flow cytometry on inguinal (draining) lymph nodes (LNs) derived from GAD-555-567 immunized HLA-DR4Transgenic (Tg) mice. Briefly, mice were immunized with 100 μg peptide in 100 μl 50% CFA/PBS subcutaneously at the base of the tail. Tissues were harvested on day 5 and single cell suspensions were analyzed by flow cytometry. LN cells were washed and incubated with 0.125 μg/ml G3H8 IgG for 1 hour at 4° C. followed by incubation with anti-human-PE as a secondary Ab (2.5 μg/ml).

As shown in FIGS. 10A-B (the results shown were obtained with IgG antibodies, but similar results were obtained with Fab antibodies, not shown), the G3H8 TCRL Ab specifically stained APCs in LNs derived from GAD immunized mice which included 6.5% positive cells (i.e., cells presenting the HLA-DR4-GAD-555-567 complexes) but not APCs presenting the HLA-DR4-HA-307-319 complex from mice immunized with the control HA-307-319 peptide.

G3H8 IgG Exhibits Enhanced Binding and Potency as Compared to the Fab—

The G3H8 IgG form was found to exhibit enhanced binding as compared to the Fab fragment (FIG. 11A). Moreover, the whole IgG TCRL molecule, which has increased avidity, inhibited GAD-specific T cell activation/function with >10-fold higher potency compared to the Fab (FIG. 11B) while maintaining its unique TCR-like specificity (FIG. 11C).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Additional References are Cited in Text

1. Svendsen, P., C. B. Andersen, N. Willcox, A. J. Coyle, R. Holmdahl, T. Kamradt, and L. Fugger. 2004. Tracking of Proinflammatory Collagen-Specific T Cells in Early and Late Collagen Induced Arthritis in Humanized Mice. J Immunol 173:7037-7045;
2. de Haard H. J., van Neer N., Reurs A., Hufton S. E., Roovers R. C., Henderikx P., de Bruine A. P., Arends J. W., Hoogenboom H. R. A large non-immunized human Fab fragment phage library that permits rapid isolation and kinetic analysis of high affinity antibodies. J. Biol. Chem., 274: 18218-18230, 1999;
3. Cohen C J, Denkberg G, Lev A, Epel M, Reiter Y. 2003. Recombinant antibodies with MHC-restricted, peptide-specific, T-cell receptor-like specificity: new tools to study antigen presentation and TCR-peptide-MHC interactions. Journal of Molecular Recognition 16:324-332;
4. Krogsgaard M., Wucherpfennig K W., Cannella B., et al. Visualization of Myelin Basic Protein (MBP) T Cell Epitopes in Multiple Sclerosis Lesions using a Monoclonal Antibody Specific for the Human Histocompatibility Leukocyte Antigen (HLA)-DR2-MBP 85-99 Complex. Journal of Experimental Medicine, vol. 191, pages 1395-1412, 2000.
5. Nepom G T, Lippolis J D, White F M, Masewicz S, Marto J A, Herman A, Luckey C J, Falk B, Shabanowitz J, Hunt D F, Engelhard V H, Nepom B S. Identification and modulation of a naturally processed T cell epitope from the diabetes-associated autoantigen human glutamic acid decarboxylase 65 (hGAD65). Proc Natl Acad Sci U S A. 2001 Feb. 13; 98(4):1763-8;
6. John A. Gebe, Susan A. Masewicz, Sharon A. Kochik, Helena Reijonen and Gerald T. Nepom. Inhibition of altered peptide ligand-mediated antagonism of human GAD65-responsive CD4 T cells by non-antagonizable T cells. Eur. J. Immunol. 2004. 34: 3337-3345;
7. Gebe J A, Unrath K A, Falk B A, Ito K, Wen L, Daniels T L, Lernmark A, Nepom G T Clin Immunol. Age-dependent loss of tolerance to an immunodominant epitope of glutamic acid decarboxylase in diabetic-prone RIP-B7/DR4 mice. Clin immunol. 2006 December; 121(3):294-304;
8. Masewicz, S. A., Papadopoulos, G. K., Swanson, E., Moriarity, L., Moustakas, A. K., and Nepom, G. T. Modulation of T cell response to hGAD65 peptide epitopes. Tissue Antigens, 59: 101-112, 2002.
9. Bach, J. M., Otto, H., Nepom, G. T., Jung, G., Cohen, H., Timsit, J., Boitard, C., and van Endert, P. M. High Affinity Presentation of an Autoantigenic Peptide in Type I Diabetes by an HLA Class II Protein Encoded in a Haplotype Protecting From Disease. Journal of Autoimmunity, 10: 375-386, 1997.
10. Ou, D., Jonsen, L. A., Metzger, D. L., and Tingle, A. J. CD4+ and CD8+ T-cell clones from congenital rubella syndrome patients with IDDM recognize overlapping GAD65 protein epitopes: Implications for HLA class I and II allelic linkage to disease susceptibility. Human Immunology, 60: 652-664, 1999.
11. Roep, B. O., Atkinson, M. A., van Endert, P. M., Gottlieb, P. A., Wilson, S. B., and Sachs, J. A. Autoreactive T cell Responses in Insulin-dependent (Type 1) Diabetes Mellitus. Report of the First International Workshop for Standardization of T cell assays. Journal of Autoimmunity, 13: 267-282, 1999.
12. Lohmann, T., Leslie, R. D., and Londei, M. T cell Clones to Epitopes of Glutamic Acid Decarboxylase 65 Raised from Normal Subjects and Patients with Insulin-dependent Diabetes. Journal of Autoimmunity, 9: 385-389, 1996.
13. Rharbaoui, Mayer, Granier, Bouanani, Thivolet, Pau, Orgiazzi, and Madec. T cell response pattern to glutamic acid decarboxylase 65 (GAD65) peptides of newly diagnosed type 1 diabetic patients sharing susceptible HLA haplotypes. Clinical & Experimental Immunology, 117: 30-37, 1999.
14. Reijonen, H., Novak, E. J., Kochik, S., Heninger, A., Liu, A. W., Kwok, W. W., and Nepom, G. T. Detection of GAD65-Specific T-Cells by Major Histocompatibility Complex Class II Tetramers in Type 1 Diabetic Patients and At-Risk Subjects. Diabetes, 51: 1375-1382, 2002.
15. Patel, S. D., Cope, A. P., Congia, M., Chen, T. T., Kim, E., Fugger, L., Wherrett, D., and Sonderstrup-McDevitt, G. Identification of immunodominant T cell epitopes of human glutamic acid decarboxylase 65_by using HLA-DR(alpha 1*0101,beta 1*0401) transgenic_mice. Proceedings of the National Academy of Sciences, 94: 8082-8087, 1997.
16. Oling, V., Marttila, J., Ilonen, J., Kwok, W. W., Nepom, G., Knip, M., Simell, O., and Reijonen, H. GAD65- and proinsulin-specific CD4+ T-cells detected by MHC class II tetramers in peripheral blood of type 1 diabetes patients and at-risk subjects. Journal of Autoimmunity, 25: 235-243, 2005.

1. An isolated complex comprising a major histocompatibility complex (MHC) class II and a type I diabetes-associated autoantigenic peptide, the isolated complex having a structural conformation which enables isolation of a high affinity entity which comprises an antigen binding domain capable of specifically binding to a native conformation of a complex composed of said MHC class II and said type I diabetes-associated autoantigenic peptide, wherein said diabetes-associated autoantigenic peptide is covalently bound at a C terminus thereof to an N-terminus of an extracellular domain of a beta chain of said MHC class II.

2. An isolated high affinity entity comprising an antigen binding domain capable of specifically binding a complex composed of a major histocompatibility complex (MHC) class II and a type I diabetes-associated autoantigenic peptide, wherein the isolated high affinity entity does not bind to said MHC class II in an absence of said diabetes-associated autoantigenic peptide, wherein the isolated high affinity entity does not bind to said diabetes-associated autoantigenic peptide in an absence of said MHC class II.

3-4. (canceled)

5. The isolated complex of claim 1, wherein said diabetes-associated autoantigenic peptide is covalently embedded between amino acids 1-6 of an extracellular domain of a beta chain of said MHC class II.

6. The isolated complex of claim 1, wherein said diabetes-associated autoantigenic peptide is flanked at a C-terminus thereof by a linker peptide.

7. The isolated complex of claim 1, wherein diabetes-associated autoantigenic peptide being translationally fused to said extracellular domain.

8. The isolated complex of claim 7, wherein said beta chain of said MHC class II comprises a first member of a binding pair which upon expression in eukaryotic cells binds to a second member of said binding pair, wherein said second member is comprised in an alpha chain of said MHC class II, wherein said beta chain and said alpha chain form said MHC class II.

9. The isolated high affinity entity of claim 2, wherein said antigen binding domain is capable of specifically binding to a native conformation of said complex composed of said MHC class II and said type I diabetes-associated autoantigenic peptide.

10. An isolated high affinity entity comprising an antigen binding domain being isolatable by the complex of claim 1.

11. An isolated high affinity entity comprising an antigen binding domain capable of specifically binding to the isolated complex of claims 1.

12. The isolated high affinity entity of claim 10, wherein said antigen binding domain of the isolated high affinity entity is capable of specifically binding to a native conformation of a complex composed of said MHC class II and said type I diabetes-associated autoantigenic peptide.

13. The isolated high affinity entity of claim 12, wherein said antigen binding domain of the isolated high affinity entity is further capable of specifically binding to the isolated complex of claim 1.

14. An isolated high affinity entity comprising complementarity determining regions (CDRs) set forth by SEQ ID NOs:171-173 and 177-179 (CDRs 1-3 of light and heavy chains of G3H8), or SEQ ID NOs:183-185 and 189-191 (CDRs 1-3 of light and heavy chains G1H12).

15. A method of isolating a high affinity entity which specifically binds to a complex composed of a major histocompatibility complex (MHC) class II and a type I diabetes-associated autoantigenic peptide, comprising:
(a) screening a library comprising a plurality of high affinity entities with the isolated complex of claim 1; and
(b) isolating at least one high affinity entity which specifically binds to the isolated complex of claim 1, and not to said MHC class II in the absence of said type I diabetes-associated autoantigenic peptide or to said type I diabetes-associated autoantigenic peptide in an absence of said MHC class II,
thereby isolating the high affinity entities which specifically bind to the complex of the MHC class II and the type I diabetes-associated autoantigenic peptide.

16. The method of claim 15, wherein the high affinity entity further specifically binds to a native conformation of the complex of the MHC class II and the type I diabetes-associated autoantigenic peptide.

17. The isolated complex of claim 1, wherein said native conformation comprises the structural conformation of said complex of said type I diabetes-associated autoantigenic peptide and said MHC class II when presented on an antigen presenting cell (APC).

18-19. (canceled)

20. The isolated complex of claim 1, wherein said diabetes-associated autoantigenic peptide is derived from a polypeptide selected from the group consisting of preproinsulin (SEQ ID NO:213), proinsulin (SEQ ID NO:223), Glutamic acid decarboxylase (GAD (SEQ ID NO:214), Insulinoma Associated protein 2 (IA-2; SEQ ID NO:215), IA-2β (SEQ ID NO:221), Islet-specific Glucose-6-phosphatase catalytic subunit-Related Protein (IGRP isoform 1 (SEQ ID NO:216), and Islet-specific Glucose-6-phosphatase catalytic subunit-Related Protein (IGRP isoform 2 (SEQ ID NO:217), chromogranin A (ChgA) (SEQ ID NO:218), Zinc Transporter 8 (ZnT8 (SEQ ID NO:219), Heat Shock Protein-60 (HSP-60; SEQ ID NO:220), Heat Shock Protein-70 (HSP-70; SEQ ID NO:271 and 224).

21. The isolated complex of claim 1, wherein said diabetes-associated autoantigenic peptide comprises the amino acid sequence selected from the group consisting of SEQ ID NOs:1-157 and no more than 30 amino acids in length.

22. The isolated complex of claim 1, wherein said diabetes-associated autoantigenic peptide is selected from the group consisting of SEQ ID NOs:1-157, 260, and 267-268.

23. The isolated complex of claim 1, wherein said diabetes-associated autoantigenic peptide is a Glutamic acid decarboxylase (GAD) autoantigenic peptide.

24-29. (canceled)

30. The isolated high affinity entity of claim 2, wherein said antigen binding domain comprises complementarity determining regions (CDRs) set forth by SEQ ID NOs:171-173 and 177-179 (CDRs 1-3 of light and heavy chains of G3H8), or SEQ ID NOs: 183-185 and 189-191 (CDRs 1-3 of light and heavy chains G1H12).

31. A molecule comprising the isolated high affinity entity of claim 2, being conjugated to a therapeutic moiety.

32. A molecule comprising the isolated high affinity entity of claim 2, being conjugated to a detectable moiety.

33. An isolated antibody comprising a multivalent form of said high affinity entity of claim 2.

34. (canceled)

35. A pharmaceutical composition comprising as an active ingredient the isolated high affinity entity of claim 2, and a pharmaceutically acceptable carrier.

36. A method of detecting presentation of a type I diabetes-associated autoantigenic peptide on a cell, comprising contacting the cell with the high affinity entity of claim 2, under conditions which allow immunocomplex formation, wherein a presence or a level above a predetermined threshold of said immunocomplex is indicative of presentation of the diabetes-associated autoantigenic peptide on the cell.

37. A method of diagnosing type 1 diabetes (T1D) in a subject, comprising contacting a cell of the subject with the high affinity entity of claim 2, under conditions which allow immunocomplex formation, wherein a presence or a level above a pre-determined threshold of said immunocomplex in or on said cell is indicative of the type 1 diabetes in the subject.

38. A method of treating type 1 diabetes (T1D), comprising administering to a subject in need thereof a therapeutically effective amount of the high affinity entity of claim 2, thereby treating the type 1 diabetes.

39. The method of claim 38, wherein said high affinity entity is capable of blocking presentation of said complex comprising said MHC class II and said type I diabetes-associated autoantigenic peptide on antigen presenting cells.

40. The method of claim 38, wherein said high affinity entity is capable of killing antigen presenting cells which display said complex comprising said MHC class II and said type I diabetes-associated autoantigenic peptide.

41. A kit for detecting presence and/or level of a complex which comprises major histocompatibility complex (MHC) class II and a type I diabetes-associated autoantigenic peptide, the kit comprising the high affinity entity of claims 2.

42. (canceled)

43. An isolated polynucleotide comprising a first nucleic acid sequence encoding an extracellular domain of an MHC class II beta chain and a second nucleic acid sequence encoding a diabetes-associated autoantigenic peptide, wherein said second nucleic acid sequence being translationally fused upstream of said first nucleic acid sequence or between the nucleic acid sequence encoding amino acids 1-6 of said extracellular domain.

44-47. (canceled)

48. The isolated complex of claim 1, wherein the isolated complex does not include a heterologous immunoglobulin attached thereto.

49. A composition of matter comprising the isolated complex of claim 1, and a functional moiety conjugated thereto.

50. A pharmaceutical composition comprising the composition of matter of claim 49 and a therapeutically acceptable carrier.

51. (canceled)

52. The isolated complex of claim 5, wherein said diabetes-associated autoantigenic peptide is covalently attached to said beta chain between the third and forth amino acids of a mature polypeptide of said MHC class II beta chain.
